SEQUENCE LISTING (109 sequences)

SEQ ID NO: 1 - DNA, 11 bp, Homo sapiens
wtatttatwww

SEQ ID NO: 2 - DNA, 13 bp, Homo sapiens
tatttatttattt

SEQ ID NO: 3 - DNA, 13 bp, Homo sapiens
aataaataaatva

SEQ ID NO: 4 - DNA, 13 bp, Homo sapiens
taaatwvataaat

SEQ ID NO: 5 - DNA, 13 bp, Homo sapiens
aataaataaataa

SEQ ID NO: 6 - DNA, 19 bp, Homo sapiens
ctcgagwhwwaaataaata

SEQ ID NO: 7 - DNA, 19 bp, Homo sapiens
  n (1)..(19): a or g or c or t/u
ctcgagtaaatwnataaat

SEQ ID NO: 8 - DNA, 12 bp, Homo sapiens
aataaataatca

SEQ ID NO: 9 - DNA, 12 bp, Homo sapiens
aataaataatga

SEQ ID NO: 10 - DNA, 13 bp, Homo sapiens
awtaaataaatwa

SEQ ID NO: 11 - DNA, 12 bp, Homo sapiens
wwwtaaataaat

SEQ ID NO: 12 - DNA, 19 bp, Homo sapiens
aataaataaataaataaat

SEQ ID NO: 13 - DNA, 25 bp, Homo sapiens
ggcggatccgggctaaataaataaa

SEQ ID NO: 14 - DNA, 28 bp, Homo sapiens
ggcggatccgggctaaatawataaatwa

SEQ ID NO: 15 - DNA, 28 bp, Homo sapiens
ggcggatccgggcaataaatawataaat

SEQ ID NO: 16 - DNA, 12 bp, Homo sapiens
vrvvrvvatgav

SEQ ID NO: 17 - DNA, 12 bp, Homo sapiens
vvvdrvbatgch

SEQ ID NO: 18 - DNA, 12 bp, Homo sapiens
vvbvrvvatggm

SEQ ID NO: 19 - DNA, 12 bp, Homo sapiens
vdbvrhvatgty

SEQ ID NO: 20 - DNA, 12 bp, Homo sapiens
bhdvmmaatgav

SEQ ID NO: 21 - DNA, 12 bp, Homo sapiens
bshmrvcatgav

SEQ ID NO: 22 - DNA, 12 bp, Homo sapiens
hbvvrvgatgad

SEQ ID NO: 23 - DNA, 12 bp, Homo sapiens
bddvrhtatgam

SEQ ID NO: 24 - DNA, 12 bp, Homo sapiens
hddvrbaatgcd

SEQ ID NO: 25 - DNA, 12 bp, Homo sapiens
vrsvrmcatgcb

SEQ ID NO: 26 - DNA, 12 bp, Homo sapiens
ssbbrmgatgcb

SEQ ID NO: 27 - DNA, 12 bp, Homo sapiens
vbdwwrtatgcm

SEQ ID NO: 28 - DNA, 12 bp, Homo sapiens
vvbvrmaatggv

SEQ ID NO: 29 - DNA, 12 bp, Homo sapiens
vvvvrscatggm

SEQ ID NO: 30 - DNA, 12 bp, Homo sapiens
bvvsrvgatggm

SEQ ID NO: 31 - DNA, 12 bp, Homo sapiens
vdbhrbtatggm

SEQ ID NO: 32 - DNA, 12 bp, Homo sapiens
drbvrmaatgty

SEQ ID NO: 33 - DNA, 12 bp, Homo sapiens
bvbmrycatgts

SEQ ID NO: 34 - DNA, 12 bp, Homo sapiens
vdbvrrgatgty

SEQ ID NO: 35 - DNA, 12 bp, Homo sapiens
dvbvwdtatgty

SEQ ID NO: 36 - DNA, 13 bp, Homo sapiens
wwwtatttatwww

SEQ ID NO: 37 - DNA, 12 bp, Homo sapiens
wwwtatttatww

SEQ ID NO: 38 - DNA, 13 bp, Homo sapiens
wwwtatttattww

SEQ ID NO: 39 - DNA, 13 bp, Homo sapiens
wwwtatttatwww

SEQ ID NO: 40 - DNA, 13 bp, Homo sapiens
wwatttatttaww

SEQ ID NO: 41 - DNA, 13 bp, Homo sapiens
atttatttattta

SEQ ID NO: 42 - DNA, 13 bp, Homo sapiens
atttatttattta

SEQ ID NO: 43 - DNA, 17 bp, Homo sapiens
atttatttatttattta

SEQ ID NO: 44 - DNA, 21 bp, Homo sapiens
atttatttatttatttattta

SEQ ID NO: 45 - DNA, 25 bp, Homo sapiens
atttatttatttatttatttattta

SEQ ID NO: 46 - DNA, 29 bp, Homo sapiens
atttatttatttatttatttatttattta

SEQ ID NO: 47 - DNA, 33 bp, Homo sapiens
atttatttatttatttatttatttatttattta

SEQ ID NO: 48 - DNA, 37 bp, Homo sapiens
atttatttatttatttatttatttatttatttattta

SEQ ID NO: 49 - DNA, 41 bp, Homo sapiens
atttatttatttatttatttatttatttatttatttattta

SEQ ID NO: 50 - DNA, 45 bp, Homo sapiens
atttatttatttatttatttatttatttatttatttatttattta

SEQ ID NO: 51 - DNA, 49 bp, Homo sapiens
atttatttatttatttatttatttatttatttatttatttatttattta

SEQ ID NO: 52 - RNA, 13 nt, Homo sapiens
wwwuauuuauwww

SEQ ID NO: 53 - RNA, 13 nt, Homo sapiens
auuuauuuauuua

SEQ ID NO: 54 - RNA, 15 nt, Homo sapiens
wwwuauuuauuuaww

SEQ ID NO: 55 - RNA, 14 nt, Homo sapiens
ucaggauuuaaaaa

SEQ ID NO: 56 - DNA, 24 bp, Homo sapiens
atgacttccaagctggccgtggct

SEQ ID NO: 57 - DNA, 25 bp, Homo sapiens
tctcagccctcttcaaaaacttctc

SEQ ID NO: 58 - DNA, 21 bp, Homo sapiens
atggatgatgatatcgccgcg

SEQ ID NO: 59 - DNA, 24 bp, Homo sapiens
ctccttaatgtcacgcacgatttc

SEQ ID NO: 60 - DNA, 11 bp, Homo sapiens
ssmamsatgrm

SEQ ID NO: 61 - DNA, 11 bp, Homo sapiens
sssrmsatgrm

SEQ ID NO: 62 - RNA, 9 nt, Homo sapiens
uuauuuauu

SEQ ID NO: 63 - RNA, 13 nt, Homo sapiens
uaauuuauuuauu

SEQ ID NO: 64 - DNA, 20 bp, Homo sapiens
gactccacaaccacgacaca

SEQ ID NO: 65 - DNA, 20 bp, Homo sapiens
tgtgtcgtggttgtggagtc

SEQ ID NO: 66 - DNA, 21 bp, Homo sapiens
gttgtaaaacgacggccagtg

SEQ ID NO: 67 - DNA, 19 bp, Homo sapiens
cacacaggaaacagctatg

SEQ ID NO: 68 - DNA, 449 bp, Homo sapiens
  n (1)..(449): a or g or c or t/u
gactccacaaccacgacacacacatacacacacacacacacacacacacacacacacacacacacacacacnatttcctgggggctctggagctcggcaaacctcccatcccagctctactaccatttagctgtgtgacctttagcaggttgtttcacatctctgagcctccatttcncccattaggcacaatgaagactgatatngttgcaagaataaattaacttgcacaccggtatgaaatggccgccttaataccttagangggagaacaccctcccccccccccaagcctctngggttctccccctgttcctttattaatctttatggcccctgttgtttcttgggtttgttgggaatttcggggntttctccctccaaaaacttgccagaaagccntttatcgtttttcgggatttcctttattttagggttggacccctatt

SEQ ID NO: 69 - DNA, 506 bp, Homo sapiens
gactccacaaccacgacacacagtaggcactcaataaatccttgaatgaattttaaaaatgaatgtgtatcactagtgcctgattcttaatctgatcagtaactgcttgatgactgaatcaatcgctgatggaaggaacaattatgcctcattcactgccgtaacatgggaaccttgcatacagcaggtgcttaataaatgctggggcacatgaaacagatgaagagggcttggttcctcccaattccccagggccaagccctgttgctggcatgcagtaggcactccattaatgctgcctgtgtgcacgtgattgcttagcaagagctggctggacaccaccccacacgagagccctctgtggcctgccaaggggtccagaggtgggggcttctcctcaaaactgcagacgcgttacgtatcggttccttatttaggtgaccactatagaattccgatatgtggtcgacaagcttctcgagcctaggctagctctagaccacact

SEQ ID NO: 70 - DNA, 569 bp, Homo sapiens
gactccacaaccacgacacaggatgtgaaggaggtcattgaatacgcacggctccggggtatccgtgtgctttgcagagtttgacactcctggccaccctttgtcctggggaccaggtatccctggattactgactcctgctaatctgggtctgagccctctggcacctttggaccagtgaatcccagtctcaataatacctatgagttcatgaacacattcttcttaaaaatcagcttctgtcttcccagatttttatcttcatcttggaggagatgaggttgatttcacctgctggaagtcccaacccagaagatcccaggactttatgaggaagaaaggcttcggtgaggacttcaagcagcgggagtcctttacatccagacgctgctggacatcgtctcttcttatggcaagggctatgtgtcgtggttgtggagtcgggctcttgtcgtcgtcatcgtgccaccggtaccgctgtcgatgtgctggcgttcgaatttagcagcagcggtttctttggtaacacccatggtggccaaagttgagcgtttattctgagcttctgc

SEQ ID NO: 71 - DNA, 569 bp, Homo sapiens; identical to SEQ ID NO: 70

SEQ ID NO: 72 - DNA, 258 bp, Homo sapiens
gactccacaaccacgacacattctggtgtaattgctctggagcaggacttggatgtcagtgtagtacagcttctccaggtgattctaatgtgtgtcgtggttgtggagtcgggctcttgtcgtcgtcatcgctgccaccggtaccgctgtcgatgtgctggcgttcgaatttagcagcagcggtttctttggtaacacccatggtgggccaaagttgaaccgttttattcctgagccttctgcaaaaagaacaacgag

SEQ ID NO: 73 - DNA, 258 bp, Homo sapiens; identical to SEQ ID NO: 72

SEQ ID NO: 74 - DNA, 797 bp, Homo sapiens
gactccacaaccacgacacactcagaaaacctcctccataaagacactggcctcaaaaaaagaacccctgtccctttcaaattagaagagaaaaggacacgaagataagtcagcagacttggcttaccctgcccagagcgcgcgcctcaaacctccttcagtgagaactgccctttttccagacagaattcctgacgtgcaccctgggctcaaggctggacaaattcctcctggagcctcctgacggaagatgtgatgttcaccgtgggggcctccctttgctcccttaaaaagggcatttcacaccaaagaaaggaattttaacctcctttcggaaaaggcctggccccctcccctggcttgggccggaacttggaaggggcaccgggatttccccagccagaaacagggcccggcttgctccgtgtcacggttcaagggctgagcaggggaggcaaggaagctggcgggtttaaaggtgaatcagttagcattgatgtgaatcagttaccactgtctttttcggcaattcagaggtgggccctgggacaagcagggaattgattcccccggggtattaaggcagcaaaaaatctgaaagaccctggagaaatctcccgttagacagacttttcaggacagcctgtgtcgtggttgtggagtcgggcttctcctcaaaactgcagacgcgttacgtatcggatccttatttaggtgacactatagaattccgatattggtcgacaagcttctcgagcgtaggctagctctagaccacacgtgtgggggcccgactc

SEQ ID NO: 75 - DNA, 797 bp, Homo sapiens; identical to SEQ ID NO: 74

SEQ ID NO: 76 - DNA, 797 bp, Homo sapiens; identical to SEQ ID NO: 74

SEQ ID NO: 77 - DNA, 464 bp, Homo sapiens
gactccaccaccacgacacagacatggaacagaaccccatacatgctattaaaaaggggaaaaaaacagaagaccgtagctacaaagctttaaatactgcatgctacgcacttcgtgggtcaagggtcgacgctgggtgggcgtggaagcaagaagggccccaacaaggccaggggcaggcaagaagctgccttcccatttttggtgaataacgtttcatgaatctaaagggtggcgttgggccaggcgccggtttcctcctcggaccacagctcctcctggatttagttgaacccgtcagtgtgtcgtggttgtggagtcgggctcttgtcgtcgtcatcgctgccaccggtaccgctgtcgatgtgctggcgttcgaatttagcagcagcggtttctttggtaacacccatggtggccaaagttgagcgtttattctgagcttctgcaaaaagaacaagcga

SEQ ID NO: 78 - DNA, 129 bp, Homo sapiens
aatgagtcatgataatgcacgtcgctatagttcatccattttcactgctgtataatattctacttaatgaattgcatggtatgacctccattggttcttctaacggctgcattatttatttatttagcc

SEQ ID NO: 79 - DNA, 230 bp, Homo sapiens
  n (1)..(230): a or g or c or t/u
acanatcaacatgaattttgaaggggatgctacaatcaaaccatagcacaggtttttacgcattttacaattttagccatgtgtagtgggaccattttatattagtgggacaattttattttgcttaagattttgtcatgtttttctatatcccaaaatgttactgagaaacatgattttaattggctatatgtataacttgtaatttatttatttagcccggatccgcc

SEQ ID NO: 80 - DNA, 119 bp, Homo sapiens
  n (1)..(119): a or g or c or t/u
ncnatattgatatttatgtgtgtgcgtaactaaaacaagtnttccaaacactgtnttatgcttcctaaatgagctgcattatatttaatttatttgatttatttatttagcccggatcc

SEQ ID NO: 81 - DNA, 281 bp, Homo sapiens
ggcggatccgggctaaatacataaataagaaaatggcgctcagtggccagaaaacagcacttccaatttctctattttgtgggacttagacaacttctatggaaaaatgggcaaatgggtctgaggtaccctacttccaggtattttttacatacgatctctccctagcctatgcttccaatgtaatccatccccaattttccttatttcccttctgtctacccctccttcttccactgggcgatgctgaaccatcttcctctgttcctatagtgagtcgt

SEQ ID NO: 82 - DNA, 710 bp, Homo sapiens
ctcactataggaacagaccgagatgagggttggcagagggagaaataagccagccacggcagtcgcttggtttcccaggtggaatgggcaaaaggagatgatgggaacctgtcccgcagtccctgcatgaccattggccctgctggcctggcgatgtgggcatcctggggttcttagggttcccagaacaaggccccaagcaagctggaacttgggtggggaagggacatgaagaagataaacagctgactgtggcttccaaggaacatcaggggccaacccccaattcctcagtgttcctactccctgggcaaaggaattgggtttgggattcaaaaattgttttaaaatttaatatttttgttccagtggatttaggaaccaacacttgtttaacataaaaaaccaatttttgtccaattcctaacaaagttagaatgtgaggaagggaatgaaacatgagtgtttgggaactgcctttgggggctgggcgtggcgtccccgcattggggtgtcttcgctctctgggctgctatgttgccccggcccaggtccccttgtgtgtgttgccagacgggcctcatggtctgctgtgcagagagaggcaggaaggatccctgaagagtcttggagaaaggttctgtgccctcaggtggggcttaccccctcgtatttataatctttatttatatatttagcccggatccgcc

SEQ ID NO: 83 - DNA, 302 bp, Homo sapiens
acgactcactataggaacagaacgytatggaaacattaaagtaggtttaatcatctcaggtgatgaatcacatttaactcttacatcacaaataaatcataagatccaagaagaaccacgtacatttaataaacactctaattacttagaatttgaagatgatcaagatgcacaagcatcacaaggtggcgatgatttatcaaatccctttggagctgatttttaaatcacataagttcaaaatctaatcacctacaacaaacaaaaccmtttaatttatatattttagccccggatccgcc

SEQ ID NO: 84 - DNA, 302 bp, Homo sapiens
  n (1)..(302): a or g or c or t/u
acgactcactataggaacagaccgatatggaaacatcttcccatttctcagaatacaatgcaggcatgggatccacacagtaacaaggcccctaagaggttgaagagaagtcatgctaacaagcgaggatgatgcagcatgaaggaaatatgaagaagctgacagtctgatgttgctgtgcaagacttatttacccagtgcaggtggaacctgacaggnagacttttnaggacctgctgtggatctaacnacaaggcagtttggnacctgtttatttnatatatttnagcccnggatccgcc

SEQ ID NO: 85 - DNA, 436 bp, Homo sapiens
  n (1)..(436): a or g or c or t/u
acgactcactataggaacagacagttatgaacccaggtgattgaatctgggaccaacatcaggcctgcttttgatctcaccagttcgtttcatgaacccgttcatcctcttcttcctttgtccttcagggcaggccttcctttgtgcttcttctatttgttcagccaacggcaaggccagtacctttgctctcatcaggacagggtcagtctgtctccctcangacaaactcactgcttttgaagttgcagtccacttgttgttgttacctgctcatccactgcggggntgtantctcctcaaaggcaaatttggcatctcaaatgaaattttngggggccccctttgttgcncatnaggananggcaggaaccattccccangttnggttcaacccaatcccgtttttcnatngttggganaaaggggaaattnt

SEQ ID NO: 86 - DNA, 346 bp, Homo sapiens
  n (1)..(346): a or g or c or t/u
acgactcactataggaacagagcaccatgaagatgagcgaatctatggggaggaaagaagtgaggtgaactccgaagattctgatattcaggaagtgctgccggtcccaaaagcttgggccancctgnggaagaaagggaaaatcggataaacatcttgccctctaggggctctctatatatacccctatgccctctcccatttgccctcctcttcagctagggctgtgcgggcccatattgcacttctggggggagacagacttattacacnggtttaaagaaaccnaattccaccacactggccggccgttacnnatggatccnacnccggtaccnancntggc

SEQ ID NO: 87 - DNA, 231 bp, Homo sapiens
acgactcactatagggaacagaggcgaatggccaaaaaaaaatgaacagaggaaaaaaaacaaacaaatggacaaaagagacagaaagttcgaaattattatagaaaataatacatgttaataaaaacatctaatgccaatttttttcaaaacttattttgatcaggtctgaaggcataagaaaacatttaatatttgagggtaatttatatatttagcccggatccgcca

SEQ ID NO: 88 - DNA, 312 bp, Homo sapiens
  n (1)..(312): a or g or c or t/u
acgaatctcactnatganngggaacagaacagaggatgaaaacattagaggtaggtttaatcatgtcagggngatgngaatcacatttaactcttacatcacaatataaatcataagatccaagaagaaccacgtacatttaataaacactctaattacttagaatttgaagatgatcaagatgcacaagcatcacaaggtggcgatgatttatcaaatccctttggagctgatttttaaatcacataagttcaaaatctaatcacctacaacaacaaaaccattaatttatttatttagcccggatycscc

SEQ ID NO: 89 - DNA, 261 bp, Homo sapiens
  n (1)..(261): a or g or c or t/u
acgactcactataggaacagataaacatgaaagttcattaagacagtgagattgggaataaatagctttttgtttttaacatttattctcagaatccaggataaaatctagtaatcttcatgggattcttcctgtccattcacgtagccatattcatgtggtcacaaagtcaagtcagctgctcatgtttattcatgtttntctgttcctatagtgagtcgtaagccgaattccagcacactggcgggccgttactartgg

SEQ ID NO: 90 - DNA, 251 bp, Homo sapiens
  n (1)..(251): a or g or c or t/u
acgactcactataggaacagataaacatgagatttggtggggacacatattcnnaaattgcatcatcaatctttgaatataaagacatccacagcaggctttatccagccaacttctttgagactctttatagagtttgaggtctagagcatatacactanaaatattcatacttcgaaaagcannataaagtggtattatcatttttccaaagttacagcagtwgtttaaggcattcatartatgaattt

SEQ ID NO: 91 - DNA, 324 bp, Homo sapiens
  n (1)..(324): a or g or c or t/u
acgactcactataggaacagataaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaarttgarcctgttgatgcccgccctgctgccgaaccgaagactgaccactccaaccaggtttctggggttgaacaaatttccgaacggaggaaatctctgaaattgaaaaatggatgccnaaattccc

SEQ ID NO: 92 - DNA, 145 bp, Homo sapiens
acgactcactataggaacagagcgccatgacaacatactaattttaataacagcattaaggggaaaaaacgtggctttggagtattctgcaatcagcctgccaactactatttctcttaatttatatatttagcccggatccgcc

SEQ ID NO: 93 - DNA, 341 bp, Homo sapiens
acgactcactataggaacagattaraatgggagtttactcatgatttggctctccgtttgtctgttattggtgtataggaatgcctgtgatttttgcacattgattttgtatctggagaatttgctgaatttgcttatcagcttaaggagattctgggctgacacgatggggttttctaaacatacaatcatgtcatctgcaaatagagacaatttgacttgctcttttcctaactgaataccctttatttatwtatttagcccggatccgccaagccgaattccagcacactggcgggcgttactagtggatccgarctcggtaccaagcttggcgttaa

SEQ ID NO: 94 - DNA, 382 bp, Homo sapiens
  n (1)..(382): a or g or c or t/u
ggcggatccgggctaaataaataaattaaaaggtgcattttttaaaatgatgctactgcaatgcaatggtttaaataccaaaaaactgagaaaatgagctgtctgtgatccagcattaaagaacatactaaaaaagagcattaatgtaaattaagtagaaaggggatcaaaaattgaactaaccaagtttgtgcagtattgtagccaggcttctaaaattagatgtagaaaatataaatagactgctttaggtagtgagccaccagtgtyccaaaaaarggaatgaaatnaagaaaaagctcagttaacttgatccaaagcycntgaataattcttcccctgcaataggtttctgctggccttgttwtaggacataacttcc

SEQ ID NO: 95 - DNA, 195 bp, Homo sapiens
acgactcactataggaacagacaaccatgaagamcattgggaargagtatgacagaacaaggaaaatgagagctctgggcacaggtagtctggtttccaaccccactacattttgccatcttgcatggttttggatccgttccttaacttctctgagtctgatttccttatttatttatttagcccggatccgcc

SEQ ID NO: 96 - DNA, 228 bp, Homo sapiens
  n (1)..(228): a or g or c or t/u
acgactcactataggaacagagtcgaatggaaacgagtggggaagatggacttgtgtgatgagagctcccccatcntacctcttttttatcaaaatatctgctctgcgaagtggagttttaatcggctcgagtctggattagtcatagcactgcagaaaagggcaagtgagaacgacaaagcagaagaatggaaattagcttatttatatatttagcccggatccgcc

SEQ ID NO: 97 - DNA, 600 bp, Homo sapiens
gactccacwaccacgacacacagcaggagcaggagccacacaggaacactccgccgtgarggcaacagatgcagctggcaacractgtccacagccacararaaacagggcagtttcatgggaacccaaaggagytytggaaccamramccttyttcaggwtaraaaggscmmmttyktcmcccmacamwgccatggscctyccctymmkttwkttaraggcmmttggarararaaaagytwatktwkattggcamacaravcccmktgaaytkttdcccbaggccttaaggaaaggtgccggaattcmacttgaamdttcsgaaggccrgggaraahramttttyatggscmcacmgggamttkgktccgraaggggcswwgaartttggaasccatccccmvgctktktargttwvcagggaagggggctkggcsccaggcsyttgtkgggraatttyscactktktaaraaaktcscaggccagcaggcttgkwtcttggaacctgcccaaacsttgcaggcctggtagcggtaaggtgtgctggaaaaggtgggtccattttcttgccagtgcagctgtgtcgtggttgtggagtc

SEQ ID NO: 98 - DNA, 126 bp, Homo sapiens
attcacgatcagatctgttaaagcaaaatttggtggatgccgtattaatcaaatacctgttattctgctttgcattattgcatacatcttttcatccattaatttatttatttagcccggatccgc

SEQ ID NO: 99 - DNA, 190 bp, Homo sapiens
ggcagcatrgaaaaggggacatgcttatccaatacaccttgctgagcttgactgccaggattctaaaacttaggtccaaatggtgagttagtgtttgcttttgtaatctaacactagttgacatcctactacagttctcttccattccacagactttctttctaatttatatatttagcccggatccgcc

SEQ ID NO: 100 - DNA, 434 bp, Homo sapiens
cttatgtatcagtgtaactgtcattttatgttttctaactattaatttcagcttttttctttgttaattgcataccgctgctttattttagatttgtttcatgtacttttataacactttttagtcttattattgatttatttgttttctttctaattttaaaattgatctacagtttaaagctatgaattgttctctctgcactgttttggcaatttcccaaaagtcttgatctggagcttttaaaaatcattcttataaaggaaattttaccaattcagtatgtatttctctttatcctaaaagtaatttcagatcaatttactttaaacttgccaggtgattgagtcttttcattttctgagtkttttattgagwgtgagtcttattgggattatactcagataccatggkcmgcaatatatactttttgg

SEQ ID NO: 101 - DNA, 17 bp, Homo sapiens
tatttatttatttattt

SEQ ID NO: 102 - DNA, 21 bp, Homo sapiens
tatttatttatttatttattt

SEQ ID NO: 103 - DNA, 25 bp, Homo sapiens
tatttatttatttatttatttattt

SEQ ID NO: 104 - DNA, 29 bp, Homo sapiens
tatttatttatttatttatttatttattt

SEQ ID NO: 105 - DNA, 33 bp, Homo sapiens
tatttatttatttatttatttatttatttattt

SEQ ID NO: 106 - DNA, 37 bp, Homo sapiens
tatttatttatttatttatttatttatttatttattt

SEQ ID NO: 107 - DNA, 41 bp, Homo sapiens
tatttatttatttatttatttatttatttatttatttattt

SEQ ID NO: 108 - DNA, 45 bp, Homo sapiens
tatttatttatttatttatttatttatttatttatttatttattt

SEQ ID NO: 109 - DNA, 49 bp, Homo sapiens
tatttatttatttatttatttatttatttatttatttatttatttattt

(19) United States
(12) Patent Application Publication
(10) Pub. No.: US 2004/0023231 A1
(43) Pub. Date: Feb. 5, 2004

(54) System for identifying and analyzing expression of are-containing genes

(76) Inventors: Khalid S. Abu-Khabar, Riyadh (SA); Bryan R.G. Williams, Cleveland, OH (US); Mathias Frevel, Wellington (NZ); Robert H. Silverman, Beachwood, OH (US)

Correspondence Address: Pamela A Docherty, Calfee Halter & Griswold, 1400 Mc Donald Investment Center, 800 Superior Ave, Cleveland, OH, 44114, US

(21) Appl. No.: 10/257,294

(22) PCT Filed: Apr. 12, 2001
(86) PCT No.: PCT/US01/11993

(51) Int. Cl. C12Q 1/68

(52) U.S. Cl. 435/6

(57) ABSTRACT

The present invention relates to a gene discovery system and gene expression systems specific for genes encoding ARE-containing mRNAs. In one aspect, the present invention relates to computational methods of selecting coding sequences of ARE-genes from databases using a one or more ARE search sequences. The ARE search sequences are from 10 to 80 nucleotides in length and comprise a sequence which is encompassed by one of the following two sequences: (a) WU/T(AU/TU/TU/TA)TWWW, SEQ ID NO. 1, wherein none or one of the nucleotides outside of the parenthesis is replaced by a different nucleotide, and wherein W represents A, U. or T; and (b) U/T(AU/TU/T/U/T)n, SEQ ID NO. 2, wherein n indicates that the search sequence comprises from 3 to 12 of the tetrameric sequences contained within the parenthesis. The method comprises extracting from the databases, those nucleic acids whose protein coding sequences are upstream and contiguous with a 3′ untranslated region (UTR) that comprises one of the ARE search sequences. The present invention also relates to methods of selectively amplifying RNA and cDNA molecules using primers derived from and complementary to the consensus 5′ sequence motifs and primers derived from and complementary to the ARE search sequence. The present invention also relates to methods of selectively amplifying ARE genes which employ a 3′ primer which is from 15 to 50 nucleotides and length and comprises from 2 to 10 pentamers having the sequence TAAAT. The pentameric sequences in the primers are either overlapping or non-overlapping. The 3′ primers are used in the reverse transcription step of the methods, the polymerase chain reaction (PCR) amplification step of the methods, or in both the reverse transcription step and the PCR amplification step of the methods. The present invention also relates to methods of making libraries which comprise portions of the ARE genes that are selectively amplified by the present methods and to methods of making microarrays which comprise probes that hybridize under stringent conditions to portions of the protein coding sequences of the ARE genes that are selectively amplified by the present methods. The present invention also relates to librairies and the microarrays that are made by such methods.

### FIELD OF THE INVENTION

The field of this invention is identification and isolation of genes; more particularly, it is computational identification of consensus nucleotide sequences common to mRNAs that contain adenylate uridylate-rich elements (AREs), and use of these consensus sequences: i) to search gene databases to identify genes containing consensus ARE sequences, and ii) to design primers, and selectively amplify and clone isolated cellular mRNAs that contain ARE sequence elements. Genes encoding ARE-containing mRNAs or unique fragments thereof are used as probes on microarrays for analysis of gene expression.

### BACKGROUND

Adenylate uridylate-rich elements (AREs) are cis-acting sequences, usually found in the 3′ untranslated region (3′UTR) of many labile mRNAs; Such ARE-containing mRNAs have relatively short half lives and are rapidly degraded after they have been transcribed. Studies have shown that certain AREs act as instability determinants (Chen and Shyu, 1995, Trends Biochem Sci, 20:465-70.). For example, the half lives of specific long-lived mRNAs were significantly decreased by inclusion of ARE sequences in the 3′UTR of such mRNAs (Shaw and Kamen, 1986, Cell, 46:659-67.). Early studies suggested the minimal necessary sequence for a functional ARE was UUAUUUAUU (Chen and Shyu, 1995, Trends Biochem Sci, 20:465-70.; Lagnado, et al., 1994, Mol Cell Biol, 14:7984-95.; Lewis, et al., 1998, J Biol Chem, 273:13781-6.; Zubiaga, et al., 1995, Mol Cell Biol, 15:2219-30.). Studies have described the binding of specific proteins to the ARE elements in mRNA and it may be that these proteins mediate the short half life of such mRNAs (Bakheet, et al., 2001, Nucleic Acids Res, 29:246-54.).

Known ARE-containing mRNAs are encoded by many early response genes that function to regulate cell proliferation and respond to exogenous agents, such as inflammatory stimuli, radiation, and viruses. Among these gene products arc proteins that participate in growth control, such as the proto-oncogene, c-fos, and the hematopoietic growth factor, granulocyte monocyte colony stimulating factor; cytokines that respond to inflammatory stimuli, such as TNF-α and IL-8; interferons, such as IFN-αand IFN-β, that are responsible for early defenses against viruses; and cellular receptors, such as tissue factor, an initiator of blood coagulation.

ARE-mediated changes in mRNA stability are important in processes that require transient responses such as cellular growth, immune response, cardiovascular toning, and external stress-mediated pathways. Abnormal expression of genes encoding ARE-containing mRNAs, by stabilization of the mRNAs for example, may cause increased concentrations of proteins encoded by such mRNAs and lead to disease. For example, removal of the ARE element of the proto-oncogene c-fos correlates with increased oncogenicity (Raymond, et al., 1989, Oncogene Res, 5:1-12). The ARE-containing Bcl-2 mRNA, encodes an anti-apoptotic protein whose increased concentrations can lead to neoplastic transformation of follicular B-cells (Capaccioli, et al., 1996, Oncogene, 13:105-15; Schiavone, et al., 2000, Faseb J, 14:174-84.). Another example of disease, possibly caused by misregulated ARE-containing mRNAs, is the chronic inflammatory arthritis and Crohn's-like inflammatory bowel disease that were detected in mice whose ARE-containing region was deleted from the TNF gene (Kontoyiannis, et al., 1999, Immunity, 10:387-98.). Chromosomal alterations led to deletion of ARE-3′UTR in the CCND1 gene (cyclin D1, PRAD1, parathyroid adenomatosis 1) that resulted in overexpression of CCND1 mRNA in mantle cell lymphoma, a deregulation event that is thought to perturb the G1-S transition of the cell cycle and thereby contributes to tumor development (Rimokh, et al., 1994, Blood, 83:3689-96.). The tumorgenicity of small neuroblastic cells correlates with overexpression of the ARE-mRNA, MYCN, and also correlated with a large amount of a p40 ELAV-protein that targets AREs and stabilizes ARE-mRNAs when compared to substrate adherent cells (Chagnovich and Cohn, 1997, Eur J Cancer, 33:2064-7.). Tumor necrosis factor (TNF-α) is a typical ARE-mRNA and, although it is both pro-inflammatory and has anti-tumor activity to specific solid cancers, there is experimental evidence that it can act as a growth factor in certain leukemias and lymphomas (Liu, et al., 2000, J Biol Chem, 275:21086-93.).

Misregulation in ARE-mRNA pathways can result in other transiently regulated biological processes being affected. The 70-year phenomenon of the Warburg effect which is the oxygen-dependent enhanced glycolysis in cancer cells has been linked to the increased constitutive expression of a novel ARE-mRNA isoform for 6-phosphofructoso-2-kinase in cancer cells and was required for tumor growth in vitro and in vivo (Chesney, et al., 1999, Proc Natl Acad Sci U S A, 96:3047-52.). In the same context of enhanced glucose metabolism in cancer, the stability of glucose transporter Glut1 mRNA has been shown to be regulated by ARE and ARE binding proteins and correlated with certain tumors including gliomas (Hamilton, et al., 1999, Biochem Biophys Res Commun, 261:646-51.). The high invasiveness of the breast cancer cell line, MDA-MB231, has been shown to be mediated by increased constitutive levels of urokinase-type plasminogen activator (uPA) due to impairment in the ARE-mediated decay of uPA mRNA (Montero and Nagamine, 1999, Cancer Res, 59:5286-93.). The increased activity of uPA and its receptor has been associated with invasiveness in a number of tumors (Reuning, et al., 1998, Int J Oncol, 13:893-906.). Interestingly, both the uPA and its receptor belong to the ARE-gene family (Bakheet, et al., 2001, Nucleic Acids Res, 29:246-54.) indicating the tightly regulated process of cell adhesiveness in normal situations. The mRNA of the transcription factor CHOP, which is involved in cell division and apoptosis in response to stress, is regulated by ARE (Ubeda, et al., 1999, Biochem Biophys Res Commun, 262:31-8.). Increased production of hematopoietic growth factors, e.g., GM-CSF, acting as autocrine growth factors, due to defects in ARE-mediated stability, may contribute to the pathogenesis of leukemia (Hoyle, et al., 1997, Cytokines Cell Mol Ther, 3:159-68.; Paul, et al., 1997, Am J Hematol, 56:79-85.). Growth-regulated alterations in the abundance of ARE-mRNA regulating proteins, AUF1 and HuR may have pleiotropic effects on the expression of many highly regulated ARE-mRNAs and this may significantly impact the onset, maintenance, and progression of the neoplastic phenotype (Blaxall, et al., 2000, Mol Carcinog, 28:76-83.).

Despite their significance, however, probably less than 100 ARE-containing mRNAs have so far been identified. Other ARE-containing genes likely exist whose misregulation may contribute to human disease. Therefore, it would be desirable to identify additional genes that encode ARE-containing mRNAs.

### SUMMARY OF THE INVENTION

The present invention relates to a gene discovery system and gene expression systems specific for genes encoding ARE-containing mRNAs. In one aspect, the present invention relates to computational methods of selecting coding sequences of ARE-genes from databases using aone or more ARE search sequnces. The ARE search sequences are from 10 to 80 nucleotides in length and comprise a sequence which is encompassed by one of the following two sequences: (a) WU/T(AU/TU/TU/TA)TWWW, SEQ ID NO. 1, wherein none or one of the nucleotides outside of the parenthesis is replaced by a different nucleotide, and wherein W represents A, U. or T; and (b) U/T(AU/TU/T/U/T)n, SEQ ID NO. 2 , wherein n indicates that the search sequence comprises from 3 to 12 of the tetrameric sequences contained within the parenthesis. The method comprises extracting from the databases, those nucleic acids whose protein coding sequences are upstream and contiguous with a 3′untranslated region (UTR) that comprises one of the ARE search sequences. Examples of such databases are mRNA databases, cDNA databases, and genomic databases, including the human genome project. The invention also relates to methods of making DNA libraries and microarrays that comprise a plurality of the nucleic acids that are selected by the computational methods. The invention also relates to the DNA libraries and microarrays that are made by such methods. In one embodiment, the microarray comprises probes that hybridize to the coding sequences of a plurality of the genes that are listed in Table 6.

The present invention also relates to a method of identifying primer sets target to the initiation region of genes whose 3′ UTR comprise ARE sequences. In one preferred embodiment, the method employs the ARE search sequences. The ARE genes are grouped into four classes or sixteen classes. The four class grouping is based upon the the nucleotide base that is attached to the 3′ end of the start codon of the ARE genes. The sixteeen class grouping is based on the nucleotide bases that are attached to both the 5′ end and the 3′ end of the start codon, ATG, of the ARE genes. Using the ARE genes that are found in the database, consensus sequences for each of the classes are determined. The consensus sequences are useful for preparing 5′ primer sets, e.g. degenerate primers, which can be used to selectively amplify full-length and partial length ARE genes.

The present invention also relates to methods of selectively amplifying RNA and cDNA molecules using primers derived from and complementary to the consensus 5′ sequence motifs and primers derived from and complementary to the ARE search sequence. Such amplified RNA and cDNA molecules comprise the full-length or partial length sequences of new ARE genes.

The present invention also relates to methods of selectively amplifying ARE genes which employ a 3′ primer which is from 15 to 50 nucleotides and length and comprises from 2 to 10 pentamers having the sequence TAAAT. The pentameric sequences in the primers are either overlapping or non-overlapping. The 3′ primers are used in the reverse transcription step of the methods, the polymerase chain reaction (PCR) amplification step of the methods, or in both the reverse transcription step and the PCR amplification step of the methods. The present invention also relates to methods of making libraries which comprise portions of the ARE genes that are selectively amplified by the present methods and to methods of making microarrays which comprise probes that hybridize under stringent conditions to portions of the protein coding sequences of the ARE genes that are selectively amplified by the present methods. The present invention also relates to libraries and the microarrays that are made by such methods.

The present invention also relates to microarrays comprising probes which hybridize under stringent conditions to the coding sequences of the genes which comprise the sequences shown in FIG. 7.

The present invention also relates to methods of using the ARE genes for generation of PCR products or oligonucleotides for use as immonpilized probes in cDNA or oligonuceotide microarray, respectively.

The present invention also relates to methods of using the microarrays of the present invention to obtain the ARE expression profile of a subject, particularly a subject with a disease such as cancer.

### BRIEF DESCRIPTION OF THE FIGURES

FIG. 1. Selection of ARE-containing cDNA by reverse transcription. Total RNA (0.5 μg) was extracted from THP-1 cells that were treated with CHX (5 μg/ml) and LPS (10 μg/ml). cDNA was synthesized from this RNA using SuperScript II with AT-P primer (WWWTAAATAAAT) at a concentration of either 15 μg/ml (lanes 2 and 3) or 25 μg/ml (lanes 4 and 5). Different RT reaction temperatures were used, 42° C. (lanes 2 and 4) and 52° C. (lanes 3 and 5). Specific PCRs for IL-8 (upper box) and β-actin (lower box) were performed using standard PCR conditions. The regular abundance of IL-8 and β-actin is shown in lane 1. Lack of DNA contamination was verified by absence of larger specific amplified products (upper arrows) or negative control containing RNA (NC).

FIG. 2. Effect of trehalose on the efficiency of specific ARE priming and reversal of abundant cDNA. Total RNA was extracted from CHX+LPS treated THP-1 cells. cDNA was synthesized using SuperScript II with TA-P primer (TAAATWVATAAAT) at a concentration of 25 μg/ml. RT was performed in the absence (lanes 1, 2 and 3) or presence of trehalose (lanes 4 and 5) at a priming annealing temperature of 60° C. Specific PCRs (cDNA input: lanes 2 and 3, 0.5 μg; lanes 4 and 5, 0.25), for IL-8 and β-actin were performed using standard PCR conditions. Lane 1 shows the regular abundance of β-actin and IL-8 at the same PCR conditions used. Upper bands are of the expected size of β-actin product, while, the lower bands are IL-8 product of the expected size. Lack of DNA contamination was verified by absence of larger specific amplified products.

FIG. 3. Effect of initial annealing temperature and number of cycles on selectivity of the discontinuous and continuous ARE-cDNA. Total RNA (1 μg) from LPS+CHX-treated THP-1 cells was extracted and subjected to RT. 40 ng cDNA was used for the ARE-cDNA PCR using the 5′ primer, Ca (Table 3), and the 3′ ARE primer using different initial annealing temperatures (4 cycles) followed by different cycles (lane 1, 20 cycles; lane 2, 25 cycles; lane 3, 30 cycles, lane 4, 35 cycles) at high annealing temperature (60° C.). Aliquots of the amplified ARE-products were subjected to a second PCR at stringent conditions specific to IL-8 (a) and TNF-α (b) in addition to β-actin to monitor selectivity of ARE amplification. Specific amplified products of IL-8 was not due to cDNA carryover from original cDNA as PCR from the amount of carryover cDNA (4 ng) failed to show detectable IL-8 and TNF-α messages at the same PCR conditions.

FIG. 4. Schematic of the RNA-ligase directed amplification of full-length coding regions of ARE-cDNA. RL oligo is a 30-mer oligonucleotide that was phosphorylated at its 5′-end and modified at its 3′-end with an amino group.

FIG. 5. Selective amplification of ARE-cDNA by RNA-ligase directed ARE-PCR (ARE-RL-PCR). Total RNA was extracted from THP-1 cells. cDNA was synthesized by SuperScript II (at two different annealing temperatures, 42° C. and 52° C.) with oligo(dT) primer followed by linking a 5′-phosphorylated and 3′-amino modified oligomer (RL oligomer) to the 3′-end of the cDNA using RNA ligase. PCR using a 5′ primer specific to the RL oligomer, and 3′primer specific to the ARE region was performed at an annealing temperature of 42.5° C. Second specific PCR for TNF-αand β-actin was performed using either 1/10 of cDNA (lanes 1 and 3) or 1/50 of cDNA (lanes 2 and 4). PCR was used with two different dNTP concentrations: 10 μM, lanes 1 and 2 and 40 μM (lane 3 and 4). Upper bands are of the expected size of TNF-α (548 bp), while lower bands indicate the size of β-actin product (838 bp), while lack of DNA contamination was verified by absence of larger bands of 1450 and 1216 bp, respectively. C indicates cDNA carryover control from the original cDNA.

FIG. 6. Test of the first generation ARE-cDNA microarray. THP-1 cells were treated with LPS (10 μg/ml) and cycloheximide (5 μg/ml). Total RNA samples (100 μg) from treated and untreated cells were labeled with Cy3 and Cy5, respectively, and hybridized to the ARE-cDNA microarray (a). (b) The average fluorescence signals of treated versus untreated samples, as measured using the GenePix 4000A scanner over duplicate spots, are plotted demonstrating two gene expression profiles in case of LPS and LPS plus CHX, the percentages of expressed ARE genes in relation to approximately 1000 cDNA in the array, and their maximum fold induction. (c) Example of the bell-shaped transient response curves characteristic of ARE-genes (approximately 100 genes) using cluster analysis using the hierarchical Ward's cluster model (SAS-JMP).

FIG. 7. DNA sequences obtained after sequencing of ARE cDNAs obtained after reverse transcription of ARE mRNA followed by either PCR of ARE sequences or RNA-ligase directed ARE-PCR.

### DETAILED DESCRIPTION OF THE INVENTION

Identification of ARE Genes

The present invention relates to computational and laboratory methods for identifying ARE genes.

Generally, the term “gene” refers to a contiguous stretch of nucleotide bases within the genome that is transcribed into an RNA, more specifically an mRNA. Such mRNA is subsequently translated into a protein. As used herein, the term can refer not only to the DNA within the genome (i.e., genomic sequences), but also to the mRNA transcribed from the DNA, and a DNA copy of the mRNA, also called “cDNA.” Such a gene has multiple sections, parts or regions, as described below (i.e., coding sequence, 3′UTR and 5′UTR). A “complete” gene comprises all of the sections. A “fragment” of a gene consists of less than all the sections. A fragment of a gene may comprise less than one entire section of a gene. A fragment of a gene that is used for the purpose of hybridization is referred to as a “probe.”

As used herein, the terms “protein coding sequence” or “coding sequence,” refer to an area of a gene (e.g., genomic DNA, mRNA or cDNA) that contains the genetic information responsible for the linear positioning of amino acids into a protein. The genetic information in such a coding region normally comprises contiguous groups of three nucleotide bases, called codons, each specifying a single amino acid within the encoded protein. Such coding sequence is said to be “full length” if it encodes a protein that is of the length and sequence normally found within a cell. Such coding sequence is said to be “partial length” if it encodes a protein that is shorter than the length of the protein normally found within a cell. Such partial length coding sequences can arise, for example, when enzymes that are used to copy DNA or RNA, do not faithfully copy the entire length of DNA or RNA being used as a template.

As used herein, “3′UTR” refers to an area of a gene, cDNA or mRNA that is located 3′ or downstream of the protein coding region of said gene, cDNA or mRNA.

As used herein, “5′UTR” refers to an area of a gene, cDNA or mRNA that is located 5′ or upstream of the protein coding region of said gene, cDNA or mRNA.

As used herein, “ARE” means “adenylate uridylate-rich element.” Such AREs are found in the 3′UTR of a gene. As used herein, an ARE gene, refers to a gene which contains an ARE within its 3′UTR.

Computational Derivation of the ARE Search Sequence

In one aspect, the present invention provides an ARE search sequences which can be used to select ARE genes from public databases. One group of ARE search sequence comprise the sequence WU/T(AU/TU/TU/TA)U/TWWW, SEQ ID NO. 1, wherein none or one of the nucleotides outside of the parenthesis is replaced by a different nucleotide, and wherein W represents A, U, or T. Another group of search sequences comprise the sequence U/T(AU/TU/TU/T)n, SEQ ID NO. 2, wherein n indicates that the search sequences comprises from 3 to 12 of the tetrameric sequences within the parenthesis. The ARE search sequences were derived through analysis of the sequences of 57 mRNAs that are known to contain ARE sequences in their 3′UTR. The two rules used to include an mRNA among the 57 mRNAs are: i) an mRNA in which the ARE sequence has been shown to control mRNA stability or half-life, or ii) an ARE-containing mRNA that is known to be transiently induced. From the 3′UTR of these 57 mRNAs, consensus ARE sequences were generated through use of multiple expectation maximization for motif elicitation (MEME) program (Bailey and Gribskov, 1998, J Comput Biol, 5:211-21.). The sequence, TATTTAWW (W=A or T) was obtained. Using the 57 sequences, a consensus analysis was then performed around the TATTTAWW motif. In one embodiment, the parameters of the analysis specify a 75% certainty of a stated nucleotide being at each position. Using these parameters, the ARE search sequences were derived.

Derivation of the mRNA Database to be Searched with the ARE Search Sequence

A total of 36,951 human mRNA/cDNA sequences were extracted from GenBank Release 113 (National Center for Biotechnology Information, NCBI). Those sequences that encode full-length open reading frames were retained and others discarded. The 3′UTR sequences were extracted from each mRNA/cDNA sequence. The sequences containing no 3′UTR were discarded. A list of 13,057 sequences remained.

Searching the mRNA Database with ARE Search Sequences

In one embodiment, the 13,057 sequences were searched for the WWWTATTTATWW sequence using the FindPattern analysis routine (Genetics Computer Group/Oxford Molecular Company; Madison, Wis.) allowing 1 bp mismatch on each side, outside of the core TATTTAT sequence. Redundant sequences were eliminated. The sequences found comprised 897 independent mRNA/cDNA sequences (see listing shown in Table 6 at end of examples).

In other embodiments of the invention, other variations of the ARE search sequence were used to search the mRNA database. Examples of the ARE search sequences which can be used include: WWWT(ATTTA)TWWW, SEQ ID NO. __, WWWT(ATTTA)TWW, SEQ ID NO. __, WWWT(ATTTA)TTWW, SEQ ID NO __, WWWT(ATTTA)TWWW. SEQ ID NO. __, WW(ATTTATTTA)WW, SEQ ID NO. __, ATTT(ATTTA)TTTA, SEQ ID NO. __, A(TTTA)ₙ, where n can be from 3 to 12. These search sequences can be further varied by allowing between 0 and 2 nucleotides outside of the nucleotides shown in parenthesis above not to match (i.e., mismatches).

Searching Genomic Databases with ARE Search Sequences

In another embodiment, ARE search sequences are used to search existing databases of genomic DNAs. A major difference between searching a genomic database as compared to searching a database comprised of 3′UTR sequences is that the ARE search sequence can be found in regions of genes other than the 3′UTR. Identification of a sequence matching the ARE search sequence within the coding region of a gene is not useful. Only ARE search sequences present in the context of the 3′UTR likely function as determinants of mRNA stability.

To determine the possibility that ARE search sequences are found in a context other than the 3′UTR of a gene, diagnostic computational tests are performed. In one test, for example, the full protein coding sequence plus 3′UTR (not just the 3′UTR) of the 13,057 mRNAs/cDNAs described above are searched for the WWWTATTTATWWW sequence. The results of this search are 897 matches, the same number as found previously, when only the 3′UTR regions of these genes are searched. This result indicates that the ARE search sequence is not found within the coding region of these genes.

In another diagnostic computational test, the ARE search sequence is searched in a database of genomic sequences from the human genome project. While the ARE search sequence is not found with significant frequency in protein coding or 5′UTR regions of genes, ARE search sequences are frequently found in introns of genes throughout the genome.

Therefore, additional computational methods are used to eliminate from consideration those genes in which the ARE search sequence is found in regions other than the 3′ UTR. These additional computational methods can also be used independently as methods of finding ARE-containing genes in genomic databases. The GENSCAN computer prediction program (Burge and Karlin, 1997, J Mol Biol, 268:78-94.) is one program used for this purpose. GENSCAN is a program that predicts the presence of genes within DNA databases using probabilistic models to detect gene structures such as exons, introns, transcriptional promoters and polyadenylation signals. Using GENSCAN, it is possible to rapidly determine whether ARE search sequences are found in regions other than the 3′UTR of genes. This eliminates genes in which the ARE search sequence is found in other areas of genes (e.g., within introns).

As an alternative to the GENSCAN program, the FGENSH program (Solovyev and Salamov, 1997, Proc Int Conf Intell Syst Mol Biol, 5:294-302; Solovyev, et al., 1995, Proc Int Conf Intell Syst Mol Biol, 3:367-75) is also used. FGENSH has been developed based on the exon recognition functions that uses linear discriminant functions for splice sites, 5′-coding, internal exon, and 3′-coding region recognition.

Once GENSCAN or FGENSH software are used to identify ARE-containing genes, 6-20 kilobase pairs of contiguous sequence upstream of the ARE sequence and 1-3 kilobase pairs of contiguous sequence downsteam of the ARE sequence are obtained. The open reading frame of the genes are obtained by analysis of these contiguous regions.

Selective Amplification of ARE mRNAs by Reverse Transcription

In addition to computational identification of ARE genes that are present in databases, laboratory methods allow identification and cloning of ARE genes that are not present in computer databases.

As a first step toward laboratory-based identification of ARE genes, cDNA is synthesized from total cellular RNA using reverse transcriptase. RNA may be total cellular RNA or mRNA. Isolation of such RNA is common to those knowledgeable in the art. Such RNA could come from cells or tissues.

In one embodiment, oligo(dT) is used as the primer in the reverse transcription reaction. Oligo(dT) hybridizes to the poly(A) tails of mRNAs during first strand cDNA synthesis. Since all mRNAs normally have a poly(A) tail, first strand cDNA is made from all mRNAs present in the reaction (i.e., there is no specificity).

In another embodiment, first strand cDNA is synthesized only from those mRNAs that contain an ARE sequence in their 3′UTR. Such selectivity is achieved by replacing oligo(dT) with degenerate universal 3′ primers that specifically hybridize to ARE sequences in the 3′UTR of such mRNAs. Such degenerate universal 3′ primers are based on the ARE search sequence derived earlier and are complementary to sequences encompassed by one or more of the search sequences. The 3′ primer are from 15 to 50 nucleotides in length and comprises from 2 to 10 pentamers having the sequence TAAAT. These pentameric sequences may be overlapping, i.e. where the fifth nucleotide in the upstream pentamer is the first nucleotide in the downstream pentamer or non-overlapping. In those cases where the primers contain nonoverlapping pentamers, the primers either are not separated, i.e. they are adjacent, or, preferably are separated by from one to five nucleotides.

Examples of 3′ primers suitable for use in the reverse transcription reaction are AATAAATAAATVA (Down-ATP). SEQ ID NO. 3, TAAATWVATAAAT (Down-TAP), SEQ ID NO. 4, AATAAATAAATAA (S-MOTIFP), SEQ ID NO. 5, CTCGAGWHWWAAATAAATA (TA-XHOP), SEQ ID NO. 6, AND CTCGAGTAAATWNATAAAT (AT-XHOP), SEQ ID NO. 7, where W=A or T, H=A or C or T, V=A or G or C, and N=A or G or C or T.

In further embodiments, additional variations of the 3′ primers may be used. Such 3′ primers include: AATAAATAATCA, SEQ ID NO. 8, AATAAATAATGA, SEQ ID NO. 9, AWTAAATAAATWA, SEQ ID NO. 10, and WWWTAAATAAAT, SEQ ID NO. 11, for example. Longer primers can be used, such as those with multiple overlapping or non-overlapping ARE pentamer elements (i.e., ATTTA). Examples of such longer primers are AATAAATAAATAAATAAAT, SEQ ID NO. 12, and GGCGGATCCGGGCTAAATAAATAAA, SEQ ID NO. 13.

Preferably, the reverse transcriptase enzyme used in the reaction is stable at temperatures above 60° C., for example, SuperScript II RT (GIBCO-BRL). However, MMLV reverse transcriptase can also be used.

In a preferred embodiment, the disaccharide, trehalose, is added to the reverse transcriptase reaction. Trehalose is a disaccharide that has been shown to stabilize several enzymes including RT at temperatures as high as 60° C. (Mizuno, et al., 1999, Nucleic Acids Res, 27:1345-9.). Trehalose addition allows the use of high temperatures in the reverse transcription reaction (e.g., as high as 60° C.). Preferably, trehalose is added to the reverse transcriptase reaction such that it is present in a final concentration of between 20 to 30%. Preferably, the reverse transcriptase reaction is then performed at a temperature between 35 to 75 C., more preferably at a temperature from between 50 to 75 C., most preferably at a temperature of 60 C.

Amplification of ARE cDNAs by PCR

To clone the cDNAs representative of new ARE-containing genes, the first strand cDNAs synthesized is designed to be specific for first strand cDNAs that contain ARE-sequences. In one embodiment this employs two primer sets, the 3′ set and the 5′ set, which are designed to selectively amplify ARE genes.

The first set of primers, the 3′ set, are similar, and could be identical, to the 3′ primers used in the aforementioned specific reverse transcription of ARE-containing mRNAs. Preferably, however, the primers of the 3′ set are longer than those used for reverse transcription and have a high percentage of GC in their sequence. Examples of the 3′ set of primers used for PCR are GGCGGATCCGGGCTAAATAWATAAATWA (MOTIF-AA), SEQ ID NO. 14, and GGCGGATCCGGGCAATAAATAWATAAAT (MOTIF-T), SEQ ID NO. 15. Other variations in sequence of these 3′ primers could be made to facilitate PCR or cloning in subsequent steps, such as inclusion of restriction enzyme cleavage sites, for example.

The second set of primers, directed to the 5′ end of the genes represented by the first strand cDNAs, are determined by computational analysis of sequences in known databases. For example, 897 mRNA/cDNA sequences that were identified as containing ARE sequences in their 3′ UTRs (these 897 genes were discussed above in the section entitled, “Searching the mRNA Database for the ARE Search Sequence.”). The region in the 5′UTR that flanked the ATG start codon for each of these 897 sequences was compared. There is some sequence conservation in all eukaryotic genes known to be present surrounding the translation start codon (Kozak, 1987, Nucleic Acids Res, 15:8125-48.; Kozak, 1987, J Mol Biol, 196:947-50.).

By analysis of this 5′region of the 897 sequences a set of four degenerate primers, or alternatively, sixteen degenerate primers is designed, such that the set of primers hybridize to 99% of the first strand cDNAs derived from the 897 mRNA/cDNA sequences (Table 4). Individual degenerate primers are selected from this list to be used in PCR. The 5′ primers are designed in such a way that they hybridize to the 5′ end of a subset of the 897 ARE genes. Therefore, to amplify all possible ARE-containing mRNAs different PCR reactions using different sets of primers are used.

Using the 3′ and 5′ primers, the PCR reaction preferably is performed using Taq polymerase and is preferably hot start PCR (i.e., adding Taq polymerase to the reaction during heating for 10 min. at 95 C.) or using anti-Taq antibody (i.e., Taq polymerase is pre-incubated with anti-Taq antibody which renders the polymerase inactive until reactivated by heating). Preferably, annealing temperature of the first four PCR cycles is between 32 and 50 C. Thereafter, the annealing temperature is raised to between 60 and 65 C. for 22 to 35 cycles. A final extension step is performed at 7 C. for 3 minutes.

RNA-Ligase Based cDNA Synthesis Followed by Specific PCR Amplification of ARE Sequences

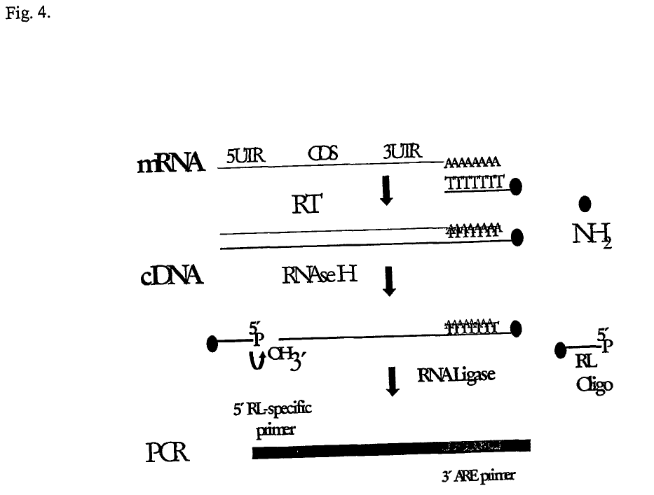

In another embodiment, synthesis of cDNA uses an RNA ligase based method, followed by amplification of such cDNAs using PCR (FIG. 4).

In such embodiment, total cellular RNA is reverse transcribed into first strand cDNA, preferably by SuperScript II reverse transcriptase and oligo(dT) primers that are modified at the 5′ ends by NH₂ (amino group prevents self ligation or inter-ligation of the oligo(dT) and the RL oligo primer). The first strand cDNA that results has the modified oligo(dT) primer incorporated and, therefore, its 5′ end blocked by NH₂ (see FIG. 4). RNase H is then used to degrade RNA in the reaction. The single-stranded, first strand cDNA that remains is then ligated to, at its 3′ end, an oligonucleotide, called the RL oligomer, that is phosphorylated at its 5′ end and protected at its 3′ end by an NH₂ group. Such RL oligomer can be from 10 to 70 nucleotides in length and is modified at its 5′ end with a phosphate group, and at its 3′end with an amino group. The sequence of such RL oligomer preferably does not have homology to human mRNAs.

Amplification of this resulting cDNA is performed by PCR using a 3′ primer containing the consensus ARE sequence, and a 5′ primer homologous to the RL oligomer.

ARE Gene Libraries

The present invention also relates to cDNA libraries that comprise the protein coding sequences of the ARE genes that are identified by the present methods. To produce such libraries, double-stranded DNA produced after PCR amplification of first strand cDNA is cloned into plasmid vectors. The cDNA may or may not be fractionated by size before cloning. Cloning of cDNA uses appropriate vectors, such as for example, T/A vectors or other cloning techniques known to those skilled in the art. Such cDNA cloning of PCR products can be accomplished through the use of commercial kits from, for example, Clontech (Palo Alto, Calif.), Invitrogen (Carlsbad, Calif.), Novagen (Madison, Wis.), Stratagene (LaJolla, Calif.), or other companies.

Library clones containing inserts are selected, further cloned, DNA extracted and purified. DNA samples are sequenced using primers specific to vector sequences flanking the inserts. Performance of these procedures is well known among those experienced in the art.

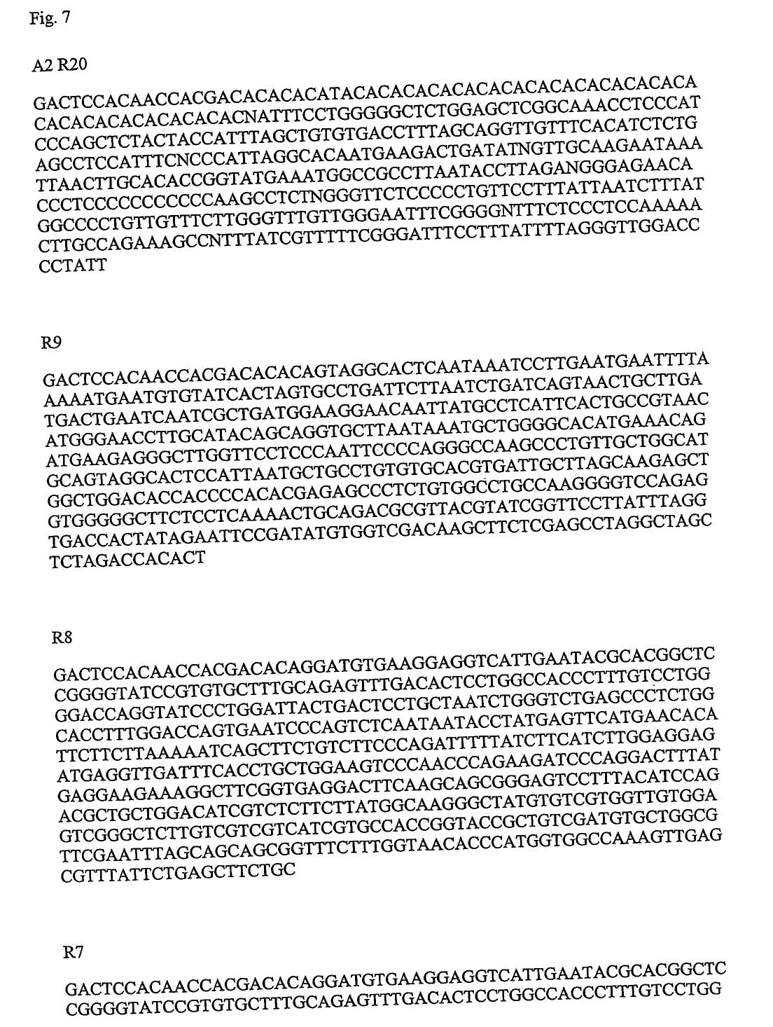

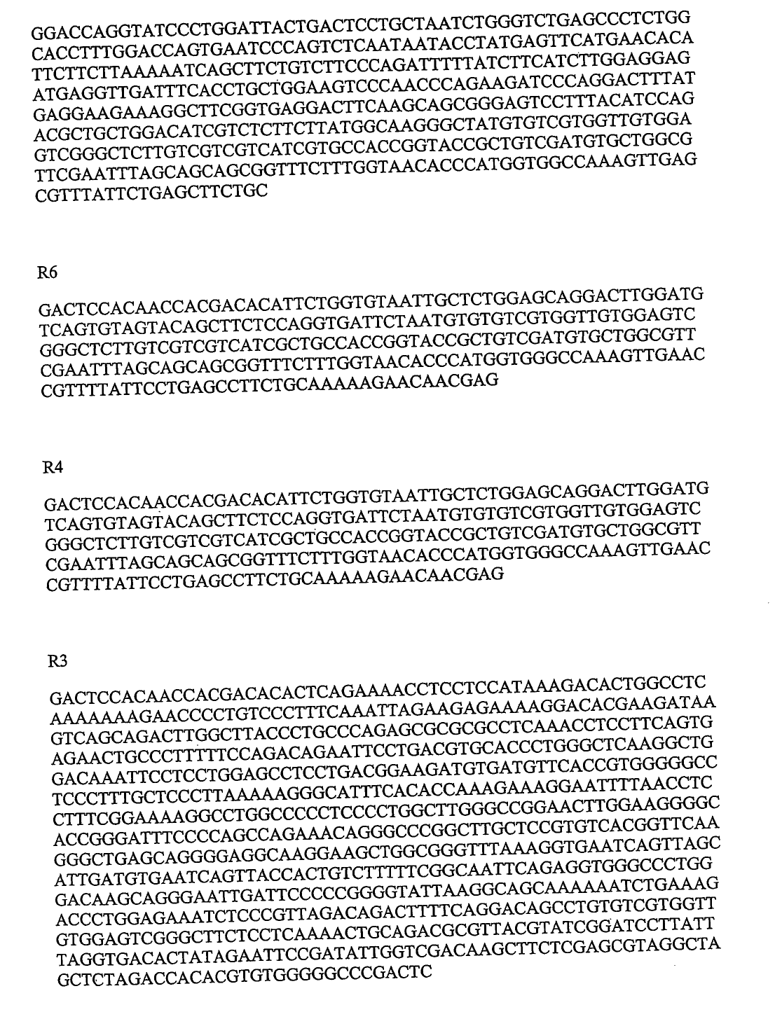

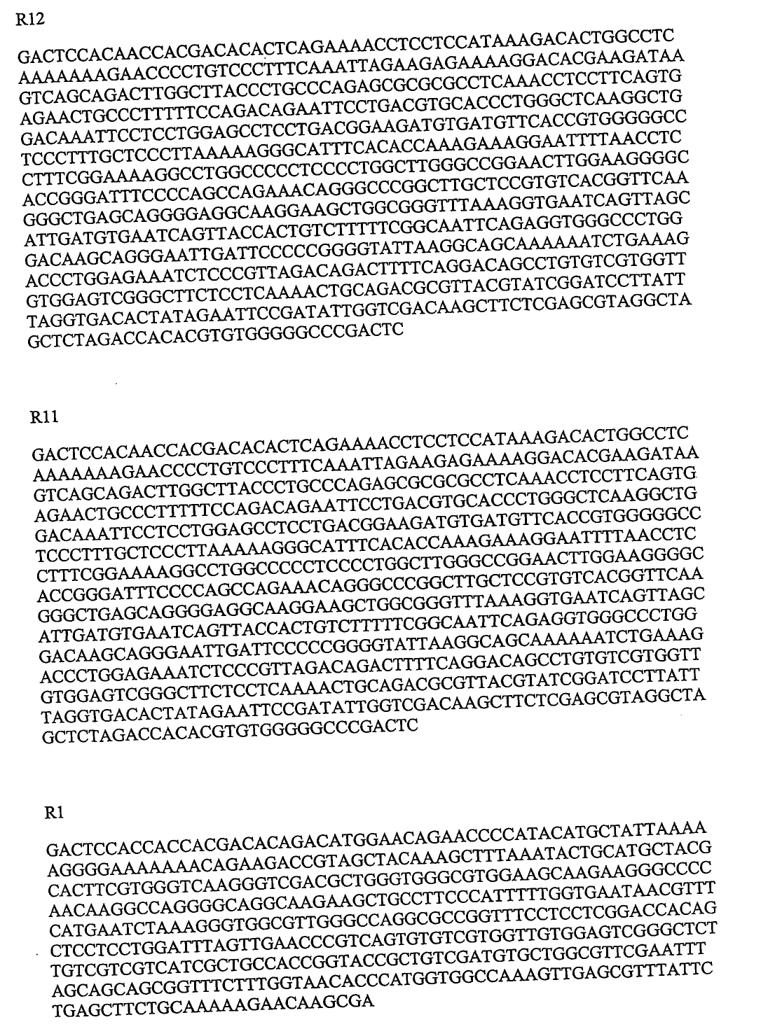

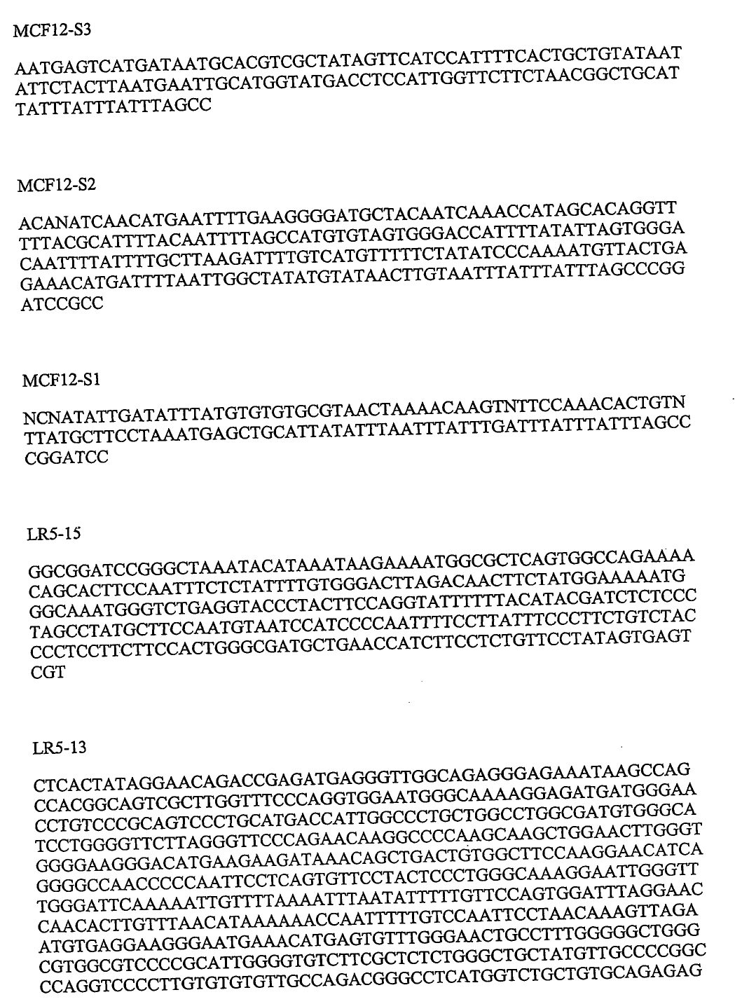

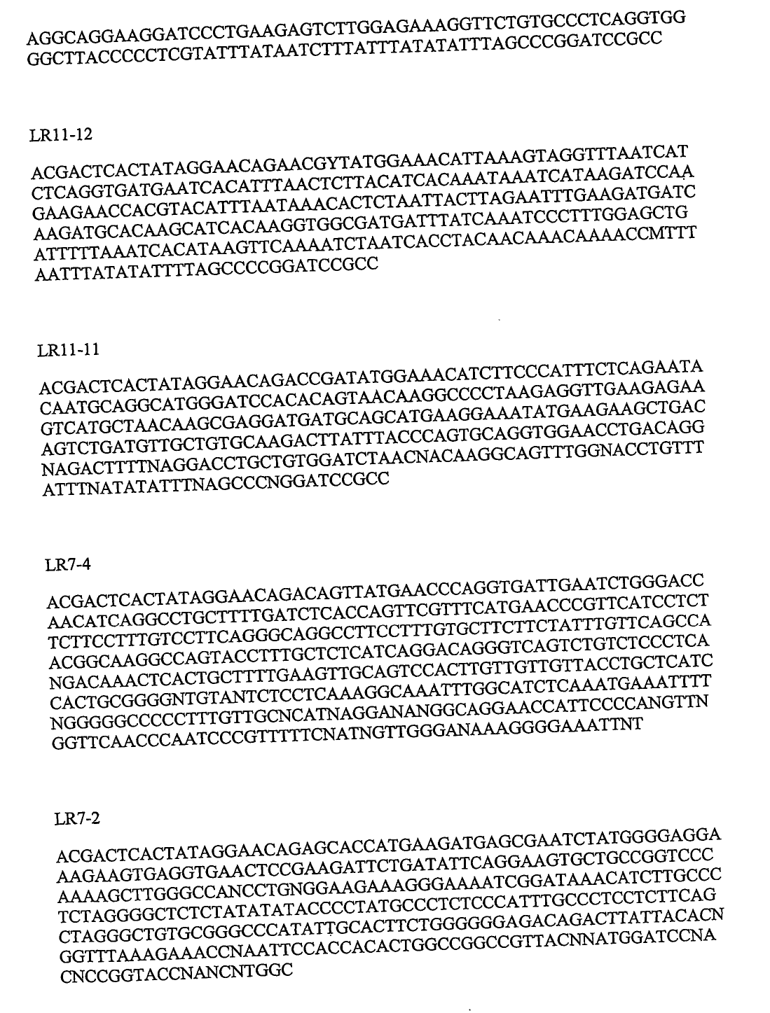

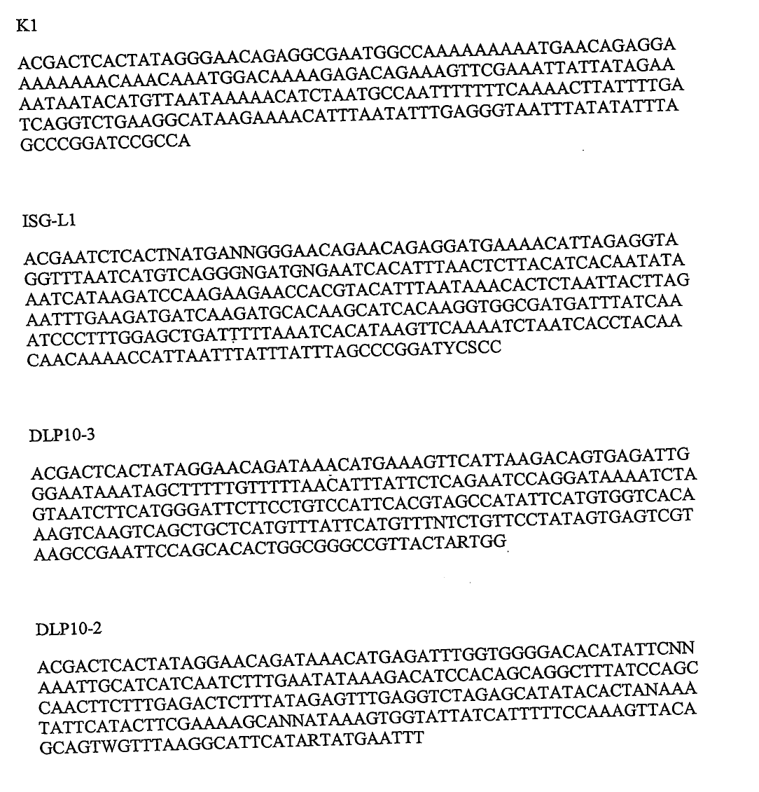

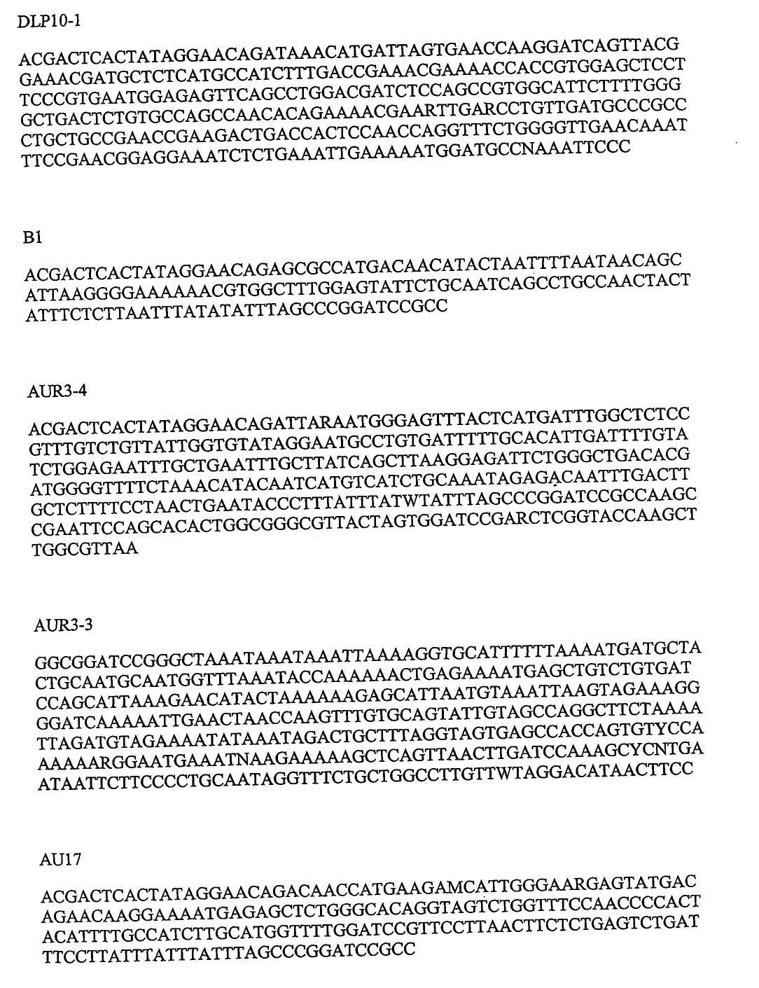

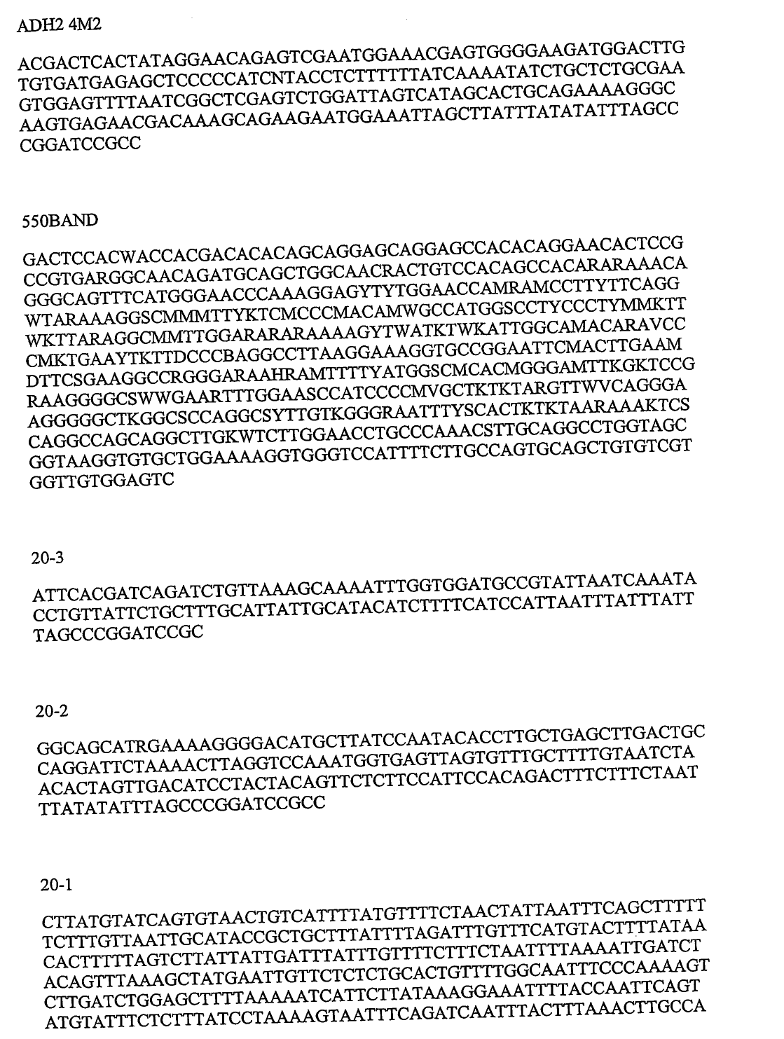

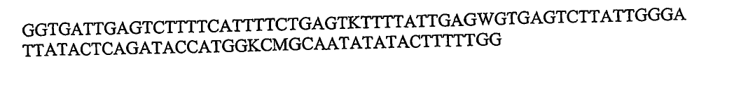

Such ARE cDNA libraries contain a plurality of DNA molecules that together represent a plurality of different ARE genes. Such individual DNA molecules normally contain a fragment of a given ARE gene. Such fragments can comprise a full length or partial length coding sequence. Such partial length coding sequences can comprise from about 10% to about 90% of the full length coding sequence. Preferably, such a partial length coding sequence comprises a unique sequence which is not contained within the protein coding sequences of genes that are not ARE-genes. The uniqueness of such sequence is determined through computational search of publicly available sequence databases. Sequences of some ARE genes isolated in this way are not found in public databases. Some such sequences are shown in FIG. 7. The library, referred to hereinafter as an “ARE library” is substantially free of nucleic acid molecules whose protein coding sequences are not part of an ARE gene. As used herein, a library is substantially free of non-ARE genes if no more than 10% of the molecules or clones that comprise the library contain coding sequences from non-ARE genes.

ARE Microarrays

The present invention also relates to microarrays that comprise probes which are nucleotide molecules derived from the nucleotide sequences of ARE genes. As used herein, the term “microarray” refers to a solid support that comprises a plurality of ARE gene probes. Preferably, fewer than 20%, more preferably fewer than 10% of the probes on the array bind under stringent hybridization conditions to the protein coding sequences of non-ARE genes. Such microarrays can comprise substantially the entire protein coding sequence of the ARE gene.

The probes that comprise the microarrays are derived from ARE genes which are identified both by computational search methods and by laboratory generation of ARE cDNA libraries as described above. The sequences derived from the ARE genes are matched to genes present in the pubically-available Unigene database (http://www.ncbi.nlm.nih.gov/UniGene/) by searching for the sequence in the BLAST database and determining the Unigene number. The Unigene database is a resource for gene discovery in which each Unigene sequence, or cluster, represents a unique gene. Clones corresponding to Unigene cluster identification numbers are used to identify clones that are then obtained from either a commercial set of 40,000 cDNA clones (human 40K set; Research Genetics; Huntsville, Ala.) or from the I.M.A.G.E. Consortium clone set (http://image.llnl.gov/).

The sources of immobilized nucleic acids (i.e., probes) placed on the microarrays may depend on the microarray and comprise several different types of probe. Such probes may comprise nucleic acids amplified from clones present in an ARE library, or obtained from Research Genetics or the I.M.A.G.E. Consortium. In such case, the insert DNAs (i.e., ARE cDNAs) from these clones are amplified by PCR using primers that hybridize to vector DNA sequences that flank the cloned insert. Alternatively, they are amplified using the 3′ primers and 5′ primer specific to the seqeuence of the cloned insert. In addition to PCR products amplified from ARE clones, probes may comprise fragments from ARE clones, such as fragments generated through restriction endonuclease cleavage of the ARE clones.

In addition, other types of molecules may be used as the gene probes in the microarrays. For example, oligonucleotides which contain at least 10 nucleotides, preferably from about 10 to about 100 nucleotides, more preferably from about 10 to about 30 nucleotides can be used. Sequence information from ARE genes is used to design and synthesize such oligonucleotides which are then placed onto the microarrays. Such oligonucleotides can be designed based on any region of an ARE-containing gene (i.e., 5′UTR, coding region, 3′UTR) as long as the sequences encoded by such oligonucleotide are unique (i.e., the sequence is not present in any other gene within the genome). Such oligonucleotides preferably have a GC ratio (i.e., the percentage of the nucleotide bases that comprise G and C) of at least 40%. Such oligonucleotides also preferably do not intemally hybridize to themselves (i.e., they do not form “hairpin” structures). In addition to oligonucleotides, other gene probes which comprise nucleobases including synthetic gene probes such as, for example, peptide nucleic acids (PNAs) can also be used.

In addition to containing sequences representative of ARE genes, microarrays will, for control purposes, also contain a smaller number of sequences representative of genes that do not contain an ARE element. Such non-ARE genes are preferably so-called “housekeeping” genes, such as for example, β-actin or GAPDH.

Microarrays are made in a variety of ways. Probes can be loaded into a robotic instrument which precisely places a predetermined amount of the probe onto the solid support. In one embodiment, probes are spotted onto glass slides that had been coated with poly-L-lysine using a SDDC-2 microarray robot (Engineering Services Inc.; Toronto, Canada), followed by UV-crosslinking and neutralization of remaining poly-L-lysine. In another embodiment, oligonucleotide probes are synthesized directly on the surface of the solid support. Making of microarrays has been described in several publications (Southern, et al., 1999, Nat Genet, 21:5-9.; Duggan, et al., 1999, Nat Genet, 21:10-4.; Cheung, et al., 1999, Nat Genet, 21:15-9.; Lipshutz, et al., 1999, Nat Genet, 21:20-4.) and U.S. Pat. Nos. 5,837,832, 6,110,426 and 6,153,743, for example). These publications and patents are incorporated herein by reference.

The ARE microarrays are then used in hybridization experiments. Hybridization of mRNA, more preferably cDNA made from mRNA, from a cell line or tissue, to a probe on the microarray is indicative of expression, at the level of transcription, of the ARE gene in the cell line or tissue that corresponds to the specific probe on the microarray. Through determination of the amount of hybridization of the cell line or tissue RNA to the totality of probes on the microarray, the expression pattern of all ARE genes comprising that cell line or tissue can be determined.

The mRNA or cDNA made from the mRNA (i.e., target nucleic acids) is normally fluorescently labeled. In one embodiment, total RNA that is to be tested for the presence and amount of ARE transcripts, is extracted from cells or tissues, labeled with Cyanine-5-dUTP (Cy5, red, Amersham; Piscataway, N.J.) in a reverse transcriptase reaction using oligo(dT)₁₁₋₁₈ primers and SuperScript II RT. Similarly, control RNA is labeled with Cyanine-3-dUTP (Cy3, green). The labeled cDNA samples are hydrolyzed by NaOH, purified by column chromatography and concentrated in TE buffer. The labeled cDNAs are mixed and hybridized to the sequences on the glass slide.

Conditions for hybridization of the target to the probe are based on the melting temperature (Tₘ) of the nucleic acid binding complex or probe, as described (Wahl, et al., 1987, Methods Enzymol, 152:399-407). The term “stringent conditions,” as used herein, is the “stringency” which occurs within a range from about Tₘ−5 (5° below the melting temperature of the probe) to about 20° C. below Tₘ. As used herein, “highly stringent” conditions employ at least 0.2×SSC buffer and at least 65° C. As recognized in the art, stringency conditions are attained by varying a number of factors such as the length and nature of the probe, the length and nature of the target sequences (i.e., the labeled cDNA), the concentration of the salts and other components, such as formamide, dextran sulfate, and polyethylene glycol, of the hybridization solution. All of these factors may be varied to generate conditions of stringency which are equivalent to the conditions listed above

In one embodiment, in addition to the labeled cDNA, the hybridization solution contains poly dA₄₀₋₆₀ (8 mg/ml), yeast tRNA (4 mg/ml), and CoT1 DNA (10 mg/ml), 3 μl of 20×SSC, and 1 μl 50× Denhardt's blocking solution. Conditions for hybrdization of such targets to the probes on the microarray are known to those experienced in the art. Such conditions have been well published. One source for such information is a series of articles in the January 1999 issue (supplement) of Nature Genetics (1999, Nat Genet, supplement, 21:1-60) which are incorporated herein by reference.

After hybridization, determination of the amount of hybridization of the target nucleic acids to individual probes on the microarray, the expression pattern of ARE genes in the cell line or tissue from which the mRNA originated is determined. In one embodiment, the glass slides are washed and read by a GenePix 4000A scanner (Axon Instruments; Foster City, Calif.) to yield gene expression data. The scanner program allows normalization of Cy3 (control sample) and Cy5 (experimental sample) ratios using the β-actin control probe on the array. The intensity ratios (Cy3 versus Cy5) represent the relative expression profile of the ARE-genes. Through comparison of such ratios for a specific gene between different samples (e.g., two different cell lines, the same cell line wherein one sample is treated with a drug compared to the other sample which is untreated, two different tissues, etc.) changes in expression of specific ARE genes are determined.

### EXAMPLES

The following examples are meant to illustrate the preferred aspects of the invention and are not to be construed as limiting the aspects of the invention in any way.

### Example 1

Computational Derivation of the ARE Motif

An ARE search sequence was defined using sequences that belonged to 57 previously identified ARE-containing mRNAs were used for the computational derivation of the ARE motif.

The selection of these mRNAs for the analysis was based on the ability of the mRNA to meet one of two criteria: i) an mRNA in which the ARE in the 3′UTR had been experimentally shown to affect the half life of that mRNA or, ii) an mRNA in which the ARE in the 3′UTR had not been experimentally shown to affect half life, but the mRNA was known to be transiently induced.

Based on these criteria, the 57 previously identified ARE-containing mRNAs that were used for this computation are: early lymphocyte activation antigen CD69 (Santis, et al., 1995, Eur J Immunol, 25:2142-6.), 6-phosphofructo-2-kinase (PFK-2)/fructose-2,6-biphosphate (Chesney, et al., 1999, Proc Natl Acad Sci U S A, 96:3047-52.), B-cell leukemia/lymphoma2 oncogene (Bcl-2) (Capaccioli, et al., 1996, Oncogene, 13:105-15), c-fos proto-oncogene (Chen, et al., 1994, Mol Cell Biol, 14:416-26.), CHOP/Growth arrest and DNA-damage inducible factor (Ubeda, et al., 1999, Biochem Biophys Res Commun, 262:31-8.), c-myb proto-oncogene (Reeves and Magnuson, 1990, Prog Nucleic Acid Res Mol Biol, 38:241-82), c-myc proto-oncogene (Brewer, 1991, Mol Cell Biol, 11:2460-6.), cyclin D1 (Rimokh, et al., 1994, Blood, 83:3689-96.), cyclooxygenase (Lasa, et al., 2000, Mol Cell Biol, 20:4265-74.), endothelin-2 (Saida, et al., 2000, Genomics, 64:51-61.), epidermal growth factor receptor (McCulloch, et al., 1998, Int J Biochem Cell Biol, 30:1265-78.), estrogen receptor α (Kenealy, et al., 2000, Endocrinology, 141:2805-13.), fibroblast growth factor 2 (Touriol, et al., 1999, J Biol Chem, 274:21402-8.), granulocyte monocyte colony stimulating factor (Reeves and Magnuson, 1990, Prog Nucleic Acid Res Mol Biol, 38:241-82; Brown, et al., 1996, J Biol Chem, 271:20108-12.), glucose transporter 1 (Hamilton, et al., 1999, Biochem Biophys Res Commun, 261:646-51.), granulocyte monocyte colony stimulating factor (Shaw and Karnen, 1986, Cell, 46:659-67.; Winzen, et al., 1999, Embo J, 18:4969-80.), gro-α (Sirenko, et al., 1997, Mol Cell Biol, 17:3898-906.), inducible nitric oxide synthase (Rodriguez-Pascual, et al., 2000, J Biol Chem, 275:26040-9.), interferon-α (Reeves and Magnuson, 1990, Prog Nucleic Acid Res Mol Biol, 38:241-82; Caput, et al., 1986, Proc Natl Acad Sci U S A, 83:1670-4.), interferon-αAA (Caput, et al., 1986, Proc Natl Acad Sci U S A, 83:1670-4.), interferon-α1 (Reeves and Magnuson, 1990, Prog Nucleic Acid Res Mol Biol, 38:241-82; Caput, et al., 1986, Proc Natl Acad Sci U S A, 83:1670-4.), interferon-α1B (Caput, et al., 1986, Proc Natl Acad Sci U S A, 83:1670-4.), interferon-αF (Reeves and Magnuson, 1990, Prog Nucleic Acid Res Mol Biol, 38:241-82; Caput, et al., 1986, Proc Natl Acad Sci U S A, 83:16704.), interferon-αG (Reeves and Magnuson, 1990, Prog Nucleic Acid Res Mol Biol, 38:241-82; Caput, et al., 1986, Proc Natl Acad Sci U S A, 83:1670-4.), interferon-αH (Reeves and Magnuson, 1990, Prog Nucleic Acid Res Mol Biol, 38:241-82; Caput, et al., 1986, Proc Natl Acad Sci U S A, 83:1670-4.), interleuldn-1α (Gorospe and Baglioni, 1994, J Biol Chem, 269:11845-51.), interferon-β (Peppel, et al., 1991, J Exp Med, 173:349-55.; Grafi, et al., 1993, Mol Cell Biol, 13:3487-93.), interferon-γ (Gillis and Malter, 1991, J Biol Chem, 266:3172-7.), interleukin-1β (Kastelic, et al., 1996, Cytokine, 8:751-61.), interleukin-10 (Kishore, et al., 1999, J Imunol, 162:2457-61.), interleukin-2 (Lindstein, et al., 1989, Science, 244:339-43.; Henics, et al., 1994, J Biol Chem, 269:5377-83.), interleukin-3 (Stoecklin, et al., 2000, Mol Cell Biol, 20:3753-63.), interleukin-4 (Reeves and Magnuson, 1990, Prog Nucleic Acid Res Mol Biol, 38:241-82), interleukin-6 (Winzen, et al., 1999, Embo J. 18:4969-80.), interleukin-8 (Winzen, et al., 1999, Embo J, 18:4969-80.), interleukin-11 (Yang and Yang, 1994, J Biol Chem, 269:32732-9.), lymphotoxin (Reeves and Magnuson, 1990, Prog Nucleic Acid Res Mol Biol, 38:241-82), K-ras proto-oncogene (Quincoces and Leon, 1995, Cell Growth Differ, 6:271-9.), leukemia inhibitory factor (Carlson, et al., 1996, Glia, 18:141-51.), macrophage colony stimulating factor (Chambers and Kacinski, 1994, J Soc Gynecol Investig, 1:310-6.), macrophage chemotaxis protein-1 (Bhattacharya, et al., 1999, Nucleic Acids Res, 27:1464-72.), macrophage inflammatory protein-α (Wang, et al., 1999, Inflamm Res, 48:533-8.), macrophage inhibitory protein-2α (Hartner, et al., 1997, Kidney Int, 51:1754-60.), Mda-7 (Madireddi, et al., 2000, Oncogene, 19:1362-8.), Monocyte Chemotactic Protein-3 (Kondo, et al., 2000, Immunology, 99:561-8.), MYCN (Chagnovich and Cohn, 1997, Eur J Cancer, 33:2064-7.), Nerve growth factor (Caput, et al., 1986, Proc Natl Acad Sci U S A, 83:1670-4.; Sherer, et al., 1998, Exp Cell Res, 241:186-93.), platelet-derived growth factor/c-sis proto-oncogene (Liang and Pardee, 1992, Science, 257:967-71.), Pim-1 proto-oncogene (Wingett, et al., 1991, J Immunol, 147:3653-9.), plasminogen activator inhibitor type 2 (Maurer, et al., 1999, Nucleic Acids Res, 27:1664-73.), thioredexin reductase (Gasdaska, et al., 1999, J Biol Chem, 274:25379-85.), tissue factor (Ahem, et al., 1993, J Biol Chem, 268:2154-9.), tumor necrosis factor (Shaw and Kamen, 1986, Cell, 46:659-67.; Zubiaga, et al., 1995, Mol Cell Biol, 15:2219-30.), urokinase-type plasminogen receptor (Montero and Nagamine, 1999, Cancer Res, 59:5286-93.), urokinase-type plasminogen activator (Montero and Nagamine, 1999, Cancer Res, 59:5286-93.) and vascular endothelial growth factor (Pages, et al., 2000, J Biol Chem, 275:26484-91.).

The 3′UTR regions of these mRNA sequences were extracted computationally using the Assemble program (Genetics Computer Group; Madison, Wis.) which extracted the sequences downstream of the coding sequence (i.e., >CDS). The 57 3′ UTRs were then analyzed by the MEME (multiple expectation maximization for motif elicitations) program which finds conserved ungapped short motifs within a group of related, unaligned sequences (Bailey and Gribskov, 1998, J Comput Biol, 5:211-21.). MEME yielded the motif pattern UAUUUAWW. Next, a consensus analysis around this motif was performed, which resulted in the pattern WWWUAUUUAUWWW (W=A or U) with a certainty level of 75% at each position (Table 1).

TABLE 1
Context Frequency Analysis around the 8-bp
MEME-derived ARE search sequence

| Base | −3 | −2 | −1 | U | A | U | U | U | A | W | W | +1 | +2 | +3 |
|---|---|---|---|---|---|---|---|---|---|---|---|---|---|---|
| A | 53 | 28 | 23 | 0 | 100 | 0 | 0 | 0 | 100 | 0 | 25 | 29 | 17 | 55 |
| T(U) | 28 | 50 | 56 | 100 | 0 | 100 | 100 | 100 | 0 | 100 | 75 | 71 | 68 | 27 |
| C | 8 | 13 | 10 | 0 | 0 | 0 | 0 | 0 | 0 | 0 | 0 | 0 | 6 | 6 |
| G | 11 | 9 | 11 | 0 | 0 | 0 | 0 | 0 | 0 | 0 | 0 | 0 | 9 | 13 |

Consensus:
AUUUAUUUAUUUA (at 50% certainty level)
WWWUAUUUAUWWWW (at 75% certainty level)

### Example 2

Determination of the Sequence Database to Search for AREs

The goal was to search a human database to identify sequences containing the ARE search sequence, WWWUAUUUAUWWW, that was determined in Example 1. To do this, the sequences to be searched had to be obtained. This was done as described below.

A total of 36,951 human mRNA/cDNA sequences were extracted from GenBank Release 113 (National Center for Biotechnology Information, NCBI) using Lookup program (Genetics Computer Group) that was used to find mRNA or cDNA in the Definition Field along with Homio sapiens in the Organism Field (Source) in GenBank entries. Subsequently, a PERL code (Practical Extraction and Report Language) was written to extract the sequences that contained the field CDS in the Features Table (indicating the sequence included a protein coding region) in order to exclude those sequences which did not have CDS. This resulted in 27,403 CDS-containing mRNA/cDNA sequences. This file was used as the input to another PERL program that extracted sequences with complete CDS (i.e., without ambiguous CDS such as <, >, complement or join). The output was 15,148 full-length CDS-containing sequences in an mRNA/cDNA file. The 3′UTRs of the sequences in this file were constructed using the Assemble program (Genetics Computer Group), which extracted the sequences downstream of CDS (i.e., >CDS). This was done in order to obtain the 3′UTR region of the genes where the ARE sequences would be found. This 3′UTR extraction step was necessary because most of the GenBank records lack the 3′UTR as an annotated Feature key, despite the fact this information can be extracted computationally from CDS Feature as executed here. The UNIX command, Stream Editor (Sed), was used to remove sequences that had no 3′UTR. A resultant list of 13,057 human full-length CDS/3′UTR-containing mRNA sequences was finally compiled.

### Example 3

Searching the Database for ARE Search Sequences

The 13-bp pattern determined in Example 1 (WWWUAUUUAUWWW ) was searched in the 13,057 sequences determined in Example 2 using FindPattern (Genetic Computer Group). The stringency was decreased by allowing one mismatch in each direction of the nucleotides flanking the core pattern (UAUUUAU), in order to allow maximum recovery from the search. This step was performed on the 3′UTRs of the full-length CDS/3′UTR-containing mRNA list. The resulting subset of sequences was made minimally redundant using the CLEANUP program (Grillo, et al., 1996, Comput Appl Biosci, 12:1-8.) with the parameters of 90% similarity and 90% overlap, which produced an output file that that contained the longest available sequences. Approximately 17% redundancy in the ARE-mRNA list was computationally removed. A total of 897 minimally redundant sequences (see listing at end of examples), approximately 8% of the human mRNA sequences analyzed, were finally obtained and subsequently termed the “ARE-mRNA database (ARED).” This database was stored as flat GenBank files and imported for farther analysis into the commercial Vector NTI software version 5.5 (InforMax; Bethesda, Md.). Each sequence in the database contained the 3′UTR, full-length CDS (i.e., protein coding sequence), and at least 10 bp of 5′UTR.

### Example 4

Testing the Specificity of the ARE Search Sequence

In Example 3, the consensus ARE sequence determined in Example 1 was used to search a database of 3′UTR sequences, as determined in Example 2. As an independent check on the specificity of the consensus ARE sequence (i.e., that it is specific to the 3′UTR), the ARE sequence was searched in the complete ARED database, which contained both 3′UTR sequences as well as coding sequences, using Assemble and FindPattern. The data show that the 13-bp ARE pattern with 2 mismatches (one on each side of the core UAUUUAU pattern) was highly selective (89% specificity) towards the 3′UTR when compared to CDS (P<0.0001). The selectivity could also be increased to 96%, although this was at the expense of losing some ARE-containing sequences (Table 2).

TABLE 2
Statistical characteristics of the computationally-derived ARE pattern in 3′UTR and CDS

| 3′UTR | | | | | CDS | | | | |
|---|---|---|---|---|---|---|---|---|---|
| No. Mismatch | No.¹ | Finds² | Mean ± S.D.³ | %⁴ | No.¹ | Finds² | Mean ± S.D.³ | % sp⁵ | P value⁶ |
| 0 | 276 | 349 | 1.3 ± 0.7 | 31% | 2 | 3 | N.A. | >99% | N.A. |
| 1 | 736 | 3670 | 4.9 ± 6 | 82% | 27 | 50 | 1.85 ± 3 | 96% | 0.0001 |
| 2 | 897 | 9781 | 10.9 ± 12 | 100% | 98 | 233 | 2.37 ± 3.7 | 89% | 0.0001 |

The ARE-mRNA list of 897 was verified against 3′UTR and CDS for the specificity and database coverage of the 13-bp pattern under different search stringency conditions (e.g., with 1 mismatch and 2 mismatches in nucleotides flanking the conserved core) used for computational compilation of the ARE-containing database.
¹No. of mRNA sequences with the 13-bp ARE search sequence present either in the 3-UTR or in the CDS (protein coding sequence) retrieved by the search..
²Indicates the number of ARE patterns found in each subset.
³Mean of finds of the 13-bp ARE pattern per 3′UTR or CDS.
⁴% Coverage = % (no. of 3′UTR with ARE pattern/total 897 mRNA sequences).
⁵% Specificity (% sp) = 1 − (CDS containing the pattern/total 897 mRNA sequences).
⁶P values indicate statistical significance between the mean of 13-bp ARE pattern per ARE mRNA using unpaired t-test with Welch correction (used because of the significantly different variances as verified by F test, P < 0.0001).
N.A = not applicable due to the small number of finds.

A distinguishable feature of the 13-bp ARE search sequence in typical ARE-mRNAs is that a significant number of ARE mRNAs (about 40% of total ARE-mRNAs) have continuous patterns of AUUUA (n>1) with the predominant pattern of WWWUAUUUAUUUAWW.

### Example 5

Mining for ARE Genes using GENSCAN

GENSCAN is a software program designed to predict complete gene structures based on a probabilistic model of the gene structure of human genomic sequences (Burge and Karlin, 1997, J Mol Biol, 268:78-94.). Such model incorporates descriptions of the basic transcriptional, translational and splicing signals, as well as length distributions and compositional features of exons, introns and intergenic regions.

There are two instances in which the GENSCAN program is used. In the first instance, GENSCAN is used to analyze the gene sequences obtained after searching a genomic database for genes containing an ARE search sequence using a program such as FindPattern. Such an analysis is used to eliminate those genes that contain the ARE consensus sequence in a region of the gene other than the 3′UTR (e.g., in an intron or intergenic regions). In the second instance, the GENSCAN program is used as an alternative to using the FindPattern analysis routine. FindPattern identifies a gene that contains a consensus ARE sequence, for example, wherever that sequence occurs within the gene. GENSCAN, however, can be used to identify only those genes in which the ARE consensus sequence occurs in the 3′UTR of the gene. GENSCAN predicts the coding segments of a genomic area. Thus, GENSCAN can be used to predict an ARE gene. First, the FindPattern program is used to locate the ARE gene upstream of the ARE region. This upstream genomic region is then subjected to GENSCAN or another computer gene prediction program to give an output of protein coding region and predicted amino acid sequence.

### Example 6

Isolation of RNA from Cells

In addition to computational identification of genes containing ARE sequences, laboratory isolation of these, as well as previously unidentified ARE-containing genes, was also performed. The first step in laboratory isolation of ARE-containing genes was isolation of RNA from cells.

In this study, the monocytic leukemia cell line, THP-1 (American Type Culture Collection; Rockville, Md.), was used. This cell line was known to produce the ARE mRNA, interleukin-8 (IL-8) and β-actin, which will be discussed later. The cells were grown in RPMI 1640 supplemented with 10% fetal bovine serum. This cell line was treated with lipopolysaccharide (LPS), an inducer of cytokines (Al-Humidan, et al., 1998, Cell Immunol, 188:12-8.), and cycloheximide (CHX), which blocks protein synthesis and increases expression of early response genes that do not require protein synthesis for transcription (Reeves and Magnuson, 1990, Prog Nucleic Acid Res Mol Biol, 38:241-82) and increases ARE-mRNA stability (Shaw and Kamen, 1986, Cell, 46:659-67.)

Total RNA was extracted from the cells using the guanidine isothiocyanate method using Tri Reagent (Molecular Research Center; Cincinnati, Ohio). The RNA was subject to DNase I treatment, followed by chloroform extraction, precipitation and resuspension in diethyl pyrocarbonate-treated (DEPC) water.

### Example 7

Selective Amplification of ARE mRNAs by Reverse Transcription

To isolate ARE genes, the isolated RNA described in Example 6 was reverse transcribed into DNA. Reverse transcription of the isolated RNA used a 13 nucleotide long degenerate primer of sequence WWWTAAATAAAT. Reverse transcription was performed in a 20 μl volume in a nuclease-free microcentrifuge tube. Total RNA (0.5 μg) was heated with different concentrations of primer to 70° C. for 10 min before quick chill on ice. Contents were collected by brief centrifugation and the following were added: 1× First Strand Buffer (250 mM Tris-Hcl, pH 8.3, 375 mM KCl, 15 mM MgCl₂), 500 μM dNTP mixture (GIBCO BRL; Gaithersburgh, Md.), 10 μM dTT (GIBCO BRL), and 20 U RNAsin (Pharmacia; Uppsala, Sweden). Contents of the tube were mixed gently and incubated at appropriate temperatures. SuperScript II (Rnase H-minus MMLV; GIBCO BRL) enzyme then was then added and incubated for two hours. The reaction was inactivated by boiling.

At this point, a pool of first strand cDNA was obtained. Because the WWWTAAATAAAT primer should have hybridized specifically to mRNAs containing ARE elements, those mRNAs should have been preferentially reverse transcribed into first strand cDNA. mRNAs that did not contain ARE elements should have been less preferentially reverse transcribed.

To test whether mRNAs containing ARE elements had been preferentially reverse transcribed, the amounts of cDNAsin the first strand cDNA pool corresponding to two sample genes was determined. The first gene, interleukin-8 (IL-8), contains discontinuous multiple nonamers, VWAUUUAWU, in its 3′UTR. IL-8, therefore, is a gene that encodes an ARE-containing mRNA. The second gene, the housekeeping gene β-actin, contains a single non-typical ARE pentamer, UCAGG(AUUUA)AAAA in its 3′UTR. β-actin, therefore, encodes an mRNA that is considered not to contain an ARE element. This is the control.

The first strand cDNA pool was used as a template for PCR amplification of IL-8 and β-actin. Determination of the ratio of PCR products of IL-8 relative to β-actin is a measure of the relative abundance of the two first strand cDNAs in the pool of cDNAs made by reverse transcription.

For amplification of IL-8 cDNA, the primers were as follows: IL-8, sense, ATGACTTCCAAGCTGGCCGTGGCT; IL-8 antisense, TCTCAGCCCTCTTCAAAAACTTCTC. For amplification of β-actin cDNA, the primers were as follows: β-actin sense; ATGGATGATGATATCGCCGCG; β-actin, antisense; CTCCTTAATGTCACGCACGATTTC. PCR was performed using 40 μg of cDNA with the following reagents in their final concentrations of: 1 unit of Taq polymerase (Perkin-Elmer), 1×PCR buffer (Perkin-Elmer), 10 μM of each of dATP, dCTP, dGTP, and dTTP, 1 μM of both sense and antisense primers. Hot start, (i.e., adding Taq polymerase to the reaction tubes during heating tubes for 10 min. at 95° C.) was used or, alternatively, Taq polymerase was pre-incubated with antibody to Taq (Sigma; St.Louis, Mo.) which rendered the Taq polymerase inactive until reactivated by heating in the first denaturation cycle. The cycling conditions were as follows: Four initial cycles of 94° C. for 1 min, 35° C. (variable temperature) for 2 min, 72° C. for 2 min; Twenty five cycles of 94° C. for 45 sec, 60° C. for 1 min, 72° C. 2 min; Final extension cycle of 72° C. for 7 min, 4° C. for overnight storage.

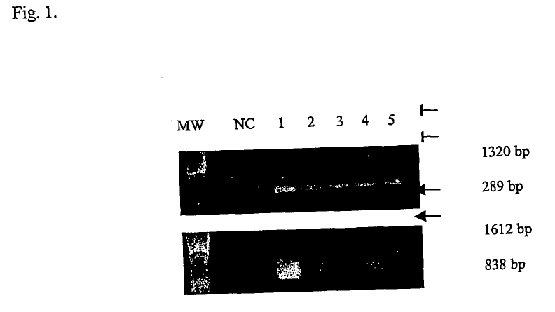

The results of this experiment are shown in FIG. 1. cDNAs made with different concentrations of primer and at different temperatures were tested. By comparing the intensities of the IL-8 bands with the intensities of the β-actin bands when moving from left to right in FIG. 1, it is seen that the ratio of IL-8 to β-actin increases. In lane 5 of FIG. 1, synthesis of cDNA from β-actin was almost completely suppressed. Under these conditions (25 μg/ml of primer and 52° C. reaction temperature ), cDNA synthesis was specific for the ARE-containing IL-8 mRNA.

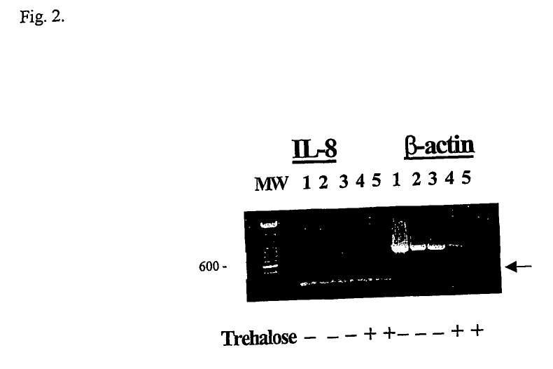

The disaccharide, trehalose, was used for further refinement for suppression of β-actin cDNA abundance while maintaining selection of ARE cDNAs (FIG. 2). RNA was mixed with ARE primers and heated in a 30% glycerol solution at 65° C. for 10 min, and cooled to 50° C. The RT buffer mix was as described above, but contained trehalose (80% w/v) and 0.1% BSA. The final concentration of trehalose in the RT reaction was approximately 20% w/v. Superscript II was added at 200 U per reaction, and the reactions were brought to an annealing temperature of 55-60° C. for 2 min. Finally, the reaction proceeded by further incubation for 1 hr until inactivated by boiling. PCR was then performed as described above.

The result of trehalose addition to the reverse transcription reactions was higher specificity of the reverse transcription reaction for the ARE-containing mRNAs as compared to reverse transcription of mRNAs that did not contain an ARE consensus sequence.

As shown in FIG. 2, the inclusion of trehalose, and thus, higher annealing temperature of as high as 60° C., resulted in dramatic suppression of abundant cDNA without affecting the less abundant IL-8 cDNA signal

### Example 8

Computational Derivation of Motifs in the 5′UTR or ARE-Containing mRNAs

In order to clone the sequences representative of ARE-containing first-strand cDNAs made in Example 7, the cDNAs were amplified. In one embodiment, this was done by PCR amplification. This PCR amplification used the 3′ primers representative of the consensus ARE sequence motif. An additional primer, derived from the 5′ region of the ARE-containing cDNA was also required. Such 5′ primers were derived from the region of the gene encompassing the translation start site of the gene, which includes the ATG start codon. Design of the 5′ primers is described in this example below.

The 5′UTR initiation context sequences (i.e., those that flank the start codon, ATG) of sequences in the ARE-mRNA database (the 897 genes described in Example 3) were analyzed. It is known that nucleotide sequences surrounding ATG start codons are conserved (Kozak, 1987, Nucleic Acids Res, 15:8125-48.; Kozalc, 1987, J Mol Biol, 196:947-50.). Thus, this region was chosen to design 5′ primers with the idea that ARE genes would have a slightly different conservation of sequences surrounding the ATG as compared to all genes.

Out of 897 ARE genes, 605 had at least 10 bp upstream (or 5′) of the ATG start codon in the database. These 605 sequences were used to examine the region around the ATG start codon. The 605 sequences were divided into either four or sixteen subsets by using the sequence designations ATGN and NATGN, respectively (N=A or C or G or T). This was followed by alignment of the truncated 5′UTR (−7bp ATG, +2bp) of the 605 sequences using the PileUP program (Genetics Computer Group). Four and sixteen consensus patterns at a certainty level of 75% at each position were derived from the alignment (Table 3). It is important to note that the consensus sequences in Table 3 are the most frequently occurring. Therefore, not every sequence in the ARED database is represented here.

The overall consensus initiation site in the ARE mRNA database was SSMAMSATGRM at a 50% certainty level at each position. In comparison, the initiation consensus of non-clustered random human sequences was SSSRMSATGRM. The conserved pattern, CACCATGG was also noted in Table 3 and appears in approximately 30% of total ARE mRNAs. It is similar to the Kozak sequence CRCCATG previously reported and to the pattern of the larger lists available at the TransTerm database¹, CAMCATGGC. ¹TransTerm is a database containing sequence information on the start and stop codons, as well as the codon usage data, for many different species. The URL is: http://uther.otago.ac.nz/Transterm.html

TABLE 3
Consensus Sequences of Initiation
Context Sequences in Human ARE mRNAs

Based on 4 divisions (ATGN):

| | |
|---|---|
| A | VRVVRVVATGAV |
| C | VVVDRVBATGCH |
| G | VVBVRVVATGGM |
| T | VDBVRHVATGTY |

Based on 16 divisions (NATGN):

| | |
|---|---|
| Aa | BHDVMMAATGAV |
| Ca | BSIHMRVCATGAV |
| Ga | HBVVRVGATGAD |
| Ta | BDDVRHTATGAM |
| Ac | IHDDVRBAATGCD |
| Cc | VRSVRMCATGCB |
| Gc | SSBBRMGATGCB |
| Tc | VBDWWRTATGCM |
| Ag | VVBVRMAATGGV |
| Cg | VVVVRSCATGGM |
| Gg | BVVSRVGATGGM |
| Tg | VDBBRBTATGGM |
| At | DRBVRMAATGTY |
| Ct | BVBMRYCATGTS |
| Gt | VDBXTRRGATGTY |
| Tt | DVBVWDTATGTY |

Truncated 5′UTRs regions of 605 ARE mRNAs that include −7bp, ATG, +2bp were stratified into either four or sixteen subsets from ARE mRNA sequences based on the formulas ATGN and NATGN, respectively. The truncated 5′UTR regions in each subset were aligned using PileUP (Genetics Computer Group) and consensus sequences were obtained using the Consensus program (Genetics Computer Group) at 75% probabilty. Letter codes follow ambiguous DNA IUB codes, e.g.,
N = A, C, G, or T.
M = A or C;
R = A or G;
W = A or T;
S = C or G;
Y = C or T;
K = G or T;
V = A or C or G;
H = A or C or T;
D = A or G or T;
B = C or G or T.

Statistical analysis of the four and sixteen 10-mer (−6 ATG, +1) consensus sequences was performed (Table 4). Sequences in each of the sixteen subsets were analyzed for initiation context sequences. Each consensus pattern contains five conserved nucleotides (i.e., ATG with one flanking nucleotide in each direction), and six additional upstream degenerate nucleotides and one additional downstream nucleotide. The most common consensus in initiation regions is Cg consensus VVVVRSCATGGM (Table 4). Other frequent initiation consensus are Ca, Ag, and Gg. Each accounts for approximately 9-10% of all ARE mRNAs.

Not all consensus sequences were unique to the initiation regions. This means that the consensus sequences could be found in areas of the mRNA sequence that did not contain the translation initiator ATG (e.g., within the protein coding sequence). Depending on the specific consensus sequence, there were varying degrees of internal sites in addition to the initiation region. The most common consensus sequence around any ATG was the Aa consensus (Table 4) which existed in 39% of the entire ARE-mRNA molecules. The least occurring consensus sequences were those flanked by a T upstream of ATG, e.g., Ta, Tc, Tg, and Tt consensus. The highest proportion of consensus in initiation regions in any subset was the Gc consensus in which 71% of the sites (initiation plus internal) were initiation sequences. The overall consensus site per mRNA ranged form 1.0 to 1.65 (i.e., >1 if the consensus sequence found in mRNAs other than at the translation initiation region).

TABLE 4
Theoretical Behavior of Amplified ARE cDNA
(a) Statistics based on Cluster Group II

| | | | | | % full |
|---|---|---|---|---|---|
| | No. | No. mRNA | | | length-CD- |
| | mRNA/ | targeted by | No. total | Site per | containing |
| | Subset¹ | 10-mer² | Sites³ | MRNA⁴ | mRNAs |
| A | 24 (24%) | 61 (62%) | 102 | 1.7 | 40% |
| C | 22 (22%) | 23 (23%) | 23 | 1 | 96% |
| G | 42 (42%) | 76 (77%) | 124 | 1.6 | 55% |
| T | 11 (11%) | 36 (36%) | 50 | 1.4 | 30% |

(b) Statistics based on Cluster Group III

| | | | | | % Full |
|---|---|---|---|---|---|
| | No. | No. mRNA | No. | | length-CD- |
| | mRNA/ | targeted by | Total | Site per | containing |
| Primer | Subset¹ | 10-mer² | Sites³ | mRNA⁴ | mRNAs |
| aA | 22 (6%) | 134 (39%) | 249 | 1.9 | 16% |
| cA | 42 (12%) | 143 (42%) | 198 | 1.4 | 29% |
| gA | 19 (6%) | 128 (38%) | 185 | 1.4 | 29% |
| tA | 14 (4%) | 81 (24% | 97 | 1.2 | 17% |
| aC | 8 (2%) | 104 (31%) | 150 | 1.4 | 8% |
| cC | 16 (5%) | 99 (29%) | 130 | 1.3 | 16% |
| gC | 22 (6%) | 94 (28%) | 114 | 1.2 | 23% |
| tC | 10 (3%) | 50 (15%) | 58 | 1.2 | 20% |
| aG | 27 (8%) | 116 (34%) | 144 | 1.2 | 23% |
| cG | 68 (20%) | 165 (49%) | 227 | 1.4 | 41% |
| gG | 34 (10%) | 130 (38%) | 173 | 1.3 | 26% |
| tG | 8 (2%) | 60 (18%) | 62 | 1 | 13% |
| aT | 12 (3%) | 47 (14%) | 48 | 1 | 25% |
| cT | 11 (3%) | 49 (14%) | 54 | 1.1 | 22% |
| gT | 8 (2%) | 85 (25%) | 109 | 1.3 | 9% |
| tT | 7 (2%) | 60 (18%) | 68 | 1.1 | 12% |

¹Number of mRNA sequences (percentage) of the total ARE-containing mRNA sequences in each of the 4 (ATGN) or 16 (NATGN) subsets.
²Number of sequences (percentage) of ARE-containing mRNA sequences in the overall ARE-containing mRNA database (i.e., includes consensus sequences found other than at the translation initiation site).
³Total number of sites (hits) in mRNA sequences in the ARED database (includes consensus sequences found other than at the translation start).
⁴Average number of hits per mRNA.
⁵% full length mRNA (i.e., the percentage of mRNAs recognized by the consensus probe that are full length mRNAs) is obtained by dividing column 2 (No. mRNA/subset) by column 4 (No. total sites). If the consensus sequence is infrequently found at sites other than the translation start, this percentage will be nearer to 100%).

### Example 9

Amplification of ARE cDNAs by PCR

Once first strand cDNA was synthesized from cellular RNA, the first strand cDNA had to be made into double-stranded DNA and the double-stranded DNA had to be amplified. In this example, amplification of the double-stranded DNA was done using PCR, 5′ primers comprising those described in Example 8 and 3′ ARE-specific primers described earlier in this application.

A PCR-protocol called ARE-cDNA PCR was used to selectively amplify ARE-cDNA. The selective amplification of ARE cDNA was verified using specific PCR to known ARE mRNA molecules with various numbers of ARE repeats (IL-8, c-fos, and TNF-α), and monitoring the abundance of the non-ARE β-actin signal, as in Example 7. TNF-α mRNA contains continuous stretches UUAUUUAUU (AUUUA)₅, while IL-8 contains discontinuous multiple nonamers in the ARE flanking region. The proto-oncogene, c-fos, has two continuous overlapping nonamers, i.e., UAAUUUAUUUAUU. As discussed earlier, β-actin, encodes an mRNA that is considered not to contain an ARE element. The goal of ARE-cDNA PCR was to amplify the typical ARE-cDNAs and concurrently suppress amplification of non-ARE sequences.

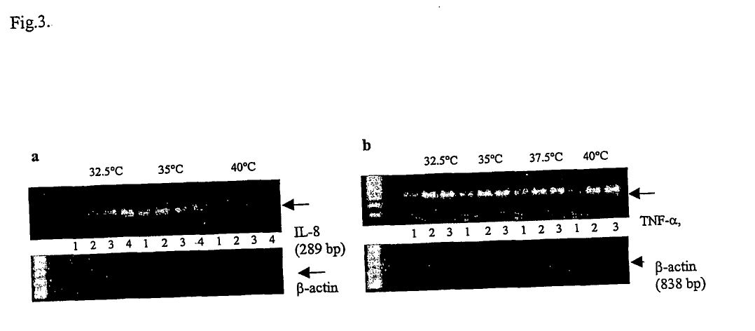

Using the optimized ARE-cDNA PCR (as described in Example 6 and as modified in the Brief Description of FIG. 3), both IL-8 and TNF-α cDNAs were specifically amplified when compared to β-actin cDNA signal (FIG. 3). FIG. 3 also shows additional data on the optimum annealing temperature and PCR cycle number. For example, small differences in ARE annealing temperatures, i.e., during the first four cycles, have significant effects on specificity in the case of IL-8 which has discontinuous multiple nonamers (FIG. 3a), but not with TNF-α which has continuous overlapping multiple nonamers (FIG. 3b). β-actin signal abundance was virtually suppressed in all lanes.

In all of the experiments, DNA contamination was monitored by lack of larger PCR products, as primers for the specific PCR were designed to span more than one exon. The specific amplifications of TNF-α and IL-8 cDNA, which were performed following ARE-cDNA PCR was not due to carryover cDNA, which has an amount of 4 ng, and was performed under high stringency conditions including the use of 50 μM of dNTP and 25 cycles.

### Example 10

RNA-Ligase Mediated Amplification Followed by Specific PCR Amplification of Sequences Containing ARE

As an alternative to selective reverse transcription or selective amplification of ARE-containing mRNAs into first strand cDNA, an alternative is RNA-ligase mediated amplification (FIG. 4).

To perform this procedure, called RL-ARE-PCR, total RNA was reversed transcribed by SuperScript II as described in Example 7 except that the primer used was oligo(dT) that had been modified at its 3′-end by the addition of NH₂. To this cDNA reaction, 2 units of RNase H were added and incubated at 37° C. for 20 min, then incubated at 90° C. for 2 min. The cDNA in the reaction was then ligated with 5′-phosphorylated and NH₂ 3′-end modified oligomers (RL oligo; Operon Technologies, Inc.; Alameda, Calif.). The 3′end of oligo(dT) and the RL oligo primer were blocked with the amino (NH₂) groups to prevent the self ligation or the inter-ligation of the oligo(dT) and RL oligomers. The 25 μl reaction contained the following: 2.5 μl of 10× ligase buffer, 16.7 μl (2 ug) of cDNA, 01.0 ul (10U) of T4 RNA ligase, 01.0 ul (0.5 ug) of the 3′-end NH₂ blocked and 5′-end phosphorylated primer. This reaction was incubated at 37° C. for 1.5 hrs, followed by incubation at 16° C. for 1.5 hrs, and then at 100° C. for 2 mins.

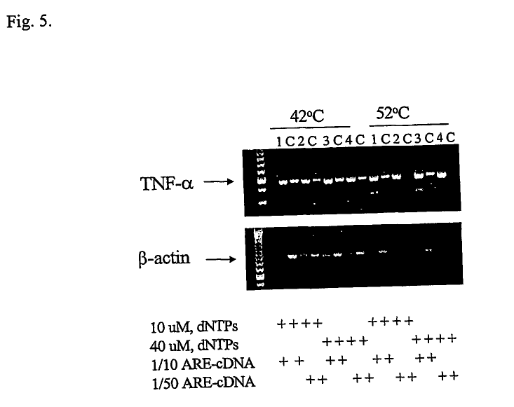

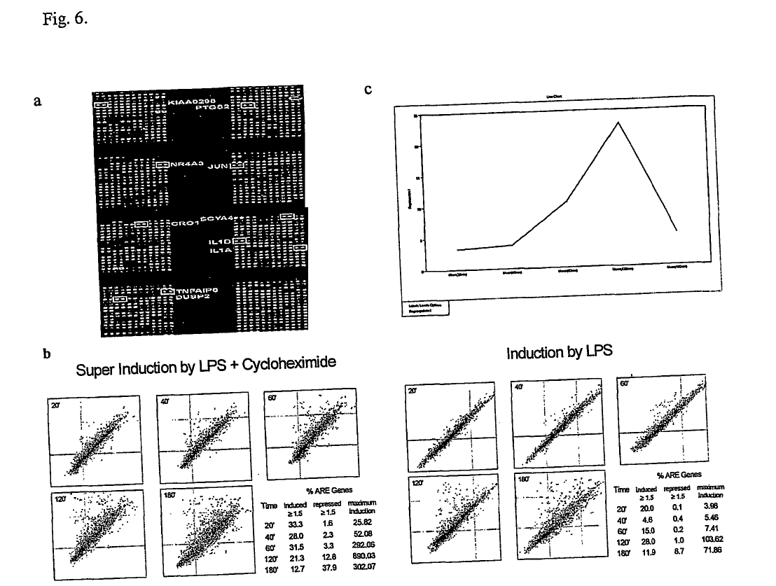

This was followed by amplification of the RL-ligated cDNA with a 5′-primer specific to the RL sequence and 3′primer specific to ARE-regions. PCR was performed as described in Example 7. The primers used for this PCR were GACTCCACAACCACGACACA and PTGTGTCGTGGTTGTGGAGTCL, where P=phosphate and L=amino linker. This PCR experiment verified amplification of the ARE-cDNA, TNF-α, but not β-actin (FIG. 5).

### Example 11

Cloning of the PCR Products

Cloning of the PCR products was needed to construct libraries of the ARE genes. A pilot construction of a pUC19 mini-library was performed using the amplified ARE-PCR products generated from the optimum conditions of RL-ARE-PCR (FIG. 5). This was done by taking the PCR products and then treating them with the Klenow fragment of DNA polymerase I and dNTPs to make the DNA ends of the PCR products blunt. The blunted ends were then phosphorylated using T4 kinase. The DNA was extracted with phenol and chloroform. The PCR products were then ligated into pUC19 plasmid vectors which had been made linear with a restriction endonuclease. Such plasmid had ends that were blunt and had been enzymatically dephosphorylated, preferably with alkaline phosphatase. The ligated plasmids were used to transform bacteria.

Bacterial colonies resulting from the transformation were randomly picked and mini-plasmid preparations were performed for evaluation purposes. The average size of the amplified inserts was 600 bp and the insert size range from 350-800 bp. This size range was satisfactory for the purpose of generating cDNA spotted probes of the microarray. The inserts of said clones were sequenced to provide DNA sequence information of said inserts. The sequences of many of these clones were found in publicly available sequence databases. The sequences of other of these clones were not found in such databases, suggesting that such clones identify previously unknown genes. The sequences of a number of such clones are shown in FIG. 7.

### Example 12

Making and using ARE Microarrays

This study describes making a microarray containing DNA sequences representative of ARE genes. Such microarrays are for use in gene expression analysis.

To make such a microarray, Unigene cluster IDs were obtained for the 897 genes in the ARE database (ARED). For genes among the 897 that had no Unigene cluster ID, and for ARE genes contained in the ARE libraries (Example 11), sequence information from those genes was used as input for BLASTN to retrieve genes corresponding to those sequences, and the corresponding Unigene cluster IDs. The Unigene cluster IDs were then used to extract the corresponding clones from the 40K set of clones of Research Genetics, Inc., which has the majority of ARE-cDNAs. In addition, individual IMAGE clones were also purchased and custom sequence-verified. Additionally; a list of 30 housekeeping genes (control genes) was compiled to be included on the array for purposes of quality control and normalization.

The cDNA clones, as glycerol culture stocks, were grown in 96-well growth blocks. The probe cDNAs that were spotted onto glass slides were obtained by PCR amplification of the insert DNAs from the clones. Purified plasmid DNA served as templates for the PCR reactions. The plasmids were prepared using commercial plasmid mini-preparation kits. All PCR reactions were carried out in 96-well thin wall PCR plates. The reaction mixtures contained 20 mM Tris-HCL (pH 8.4), 50 mM KCl, 1.5 mM MgCl₂, 0.8 mM of each dATP, dGTP, dTTP, and dCTP, 0.1 μM forward oligonucleotide primer (5′GTTGTAAAACGACGGCCAGTG), 0.1 μM reverse oligonucleotide primer (5′CACACAGGAAACAGCTATG), and 5 units Taq DNA polymerase. The reactions had a total volume of 100 μl, and contained 100-300 ng of purified plasmid to provide the template DNA. PCRs were performed using the following thermal cycler program: 1 cycle of 94 C. for 2 min, 27 cycles of 94 C. for 30 sec, 55 C. for 30 sec, and 72 C. for 2.5 min, 1 cycle of 72 C. for 5 min. The PCR products (5 μl of the reaction) were then analyzed by agarose gel electrophoresis and could be stored at −20 C. until further processing. The PCR products were further processed in 96-well format either by ethanol precipitation or using commercially available DNA purification plates. Purified or precipitated PCR products were resuspended in a salt solution (e.g. 3×SSC).

These resuspended DNAs were the probe DNAs that were spotted onto glass slides to give the ARE-containing gene array. The slides were first coated with poly-L lysine. The poly-L-lysine slide coating procedure was as follows. A batch of plain Gold Seal microscope slides was incubated in cleaning solution (2.5 M NaOH in 60% ethanol) under agitation for two hours. Subsequently, the slides were rinsed with distilled water five times, each rinse lasting 5 minutes. The slides were then incubated in poly-L-lysine solution (0.01% poly-L-lysine in 0.1× standard tissue culture PBS) for one hour under agitation. Slides were then rinsed in distilled water for one minute, and any free liquid was removed by centrifugation of the slides at low speed. The coated slides were stored dust free and could be used for array printing for several weeks.

The probe DNAs were arrayed onto the slides using a SDDC-2 microarray robot from ESI (Engineering Services Inc.; Toronto, Canada). The setup used eight print-pins, delivering eight individual probe DNAs simultaneously to each slide, and washing the pins twice in water between every probe pick-up step. The probe DNAs were contained in 384-well plates to minimize loss by evaporation during the printing procedure. The size of the array area on each slide depended on the number of probe DNAs in the array. The distance between the centers of neighboring DNA spots was 200 μm. All probe DNAs were spotted onto each array at least in duplicate. For example, an array of 1000 genes (hence 2000 array spots) printed from a 384-well plate using eight print-pins will covered an area on the slide of approximately 170 mm². After the printing, the array slides were stored dust free for 2-4 days before UV cross-linking.

The arrayed probe DNA was cross linked to the poly-L-lysine coat using a Stratalinker (Stratagene) with a UV dose of 450 mJ. The positive charges of the lysine residues on the array slides were neutralized by incubating the slides in a freshly prepared solution of 1.7% succinic anhydride in 1-methyl-2-pyrrolidinone/77 mM borate buffer for 30 minutes. The slides were then submerged for two minutes in first, distilled water of 95 C., and second 95% ethanol. Excess ethanol was then removed by centrifugation at low speed, and the cDNA microarray was stored dust free at room temperature ready to be used for hybridization.

To use the ARE microarrays for gene expression experiments, total RNA (100 ug) samples were extracted from THP-1 cells that were previously treated with CHX and LPS using the Qiagen Rneasy RNA purification kit and refined by Trizol reagent (GibcoBRL). The RNA samples were labeled with Cyanine-3-dUTP (Cy3, green) and Cyanine-5-dUTP (Cy5, red, Amersham), in two separate RT reactions using olig(dT)₁₁₋₁₈ primers and SuperScript II RT. The labeled cDNA samples were hydrolyzed by NaOH and purified on Micro Bio-Spin® 6 chromatography column (Bio-Rad) and concentrated in TE buffer. The labeled cDNA sample mixture was hybridized to the microarray. The hybridization solution contained poly dA₄₀₋₆₀ (8 mg/ml), yeast tRNA (4 mg/ml), and CoT1 DNA (10 mg/ml), 3 μl of 20×SSC, and 1 μl 50× Denhardt's blocking solution. This mixture was applied to the ARE-cDNA glass slides and hybridized under stringent conditions. Subsequently, the glass slides were washed.

Analysis of hybridization to the microarray used scanning of the microarray with a GenePix 4000A scanner (Axon Instruments). The scanner program allowed normalization of Cy3 (THP-1 control sample) and CyS (LPS+CHX treated TBP-1 sample) ratios using the β-actin control on the array. Most of the duplicates gave similar readings. The intensity ratios from two cDNA samples measured using the ARE-cDNA microarray represented the relative expression profile of the ARE genes in the two starting RNA samples. FIG. 6 shows the expression profile of the ARE-cDNA array showing the differential expression of many ARE-cDNAs (FIGS. 6a, 6b). The results supported the ARE functionality, (i) a large proportion were induced at early time points (20 min., FIG. 6b), (ii) many displayed a transient expression pattern (FIG. 6c), (iii) a large proportion were independent of protein synthesis (CHX treatment), and (iv) a large proportion were upregulated with CHX treatment.

TABLE 6
List of ARE-containing mRNA sequences and accession numbers

| | |
|---|---|
| A12140 | H. sapiens IFN-omega 1 gene. |
| A18397 | Human uPA cDNA. |
| A18757 | u-PA receptor. |
| A19048 | H. sapiens mRNA for thromboplastin (clone 2b-Apr5). |
| A21239 | H. sapiens BTA 1916 mRNA for Pai-2. |
| A21240 | H. sapiens BTA 1922 mRNA for Pai-2. |
| A26481 | Human NPY receptor Y1 gene cDNA. |
| A30262 | H. sapiens beta-casein cDNA. |
| A35395 | H. sapiens u-PA cDNA sequence. |
| AB000220 | Homo sapiens mRNA for semaphorin E, complete cds. |
| AB000509 | Homo sapiens mRNA for TRAF5, complete cds. |
| AB001106 | Homo sapiens mRNA for glia maturation factor, complete cds. |
| AB001466 | Homo sapiens mRNA for Efs1, complete cds. |
| AB001467 | Homo sapiens mRNA for Efs2, complete cds. |
| AB002292 | Human mRNA for KIAA0294 gene, complete cds. |
| AB002303 | Human mRNA for KIAA0305 gene, complete cds. |
| AB002311 | Human mRNA for KIAA0313 gene, complete cds. |
| AB002314 | Human mRNA for KIAA0316 gene, complete cds. |
| AB002329 | Human mRNA for KIAA0331 gene, complete cds. |
| AB002343 | Human mRNA for KIAA0345 gene, complete cds. |
| AB002350 | Human mRNA for KIAA0352 gene, complete cds. |
| AB002371 | Human mRNA for KIAA0373 gene, complete cds. |
| AB002372 | Human mRNA for KIAA0374 gene, complete cds. |
| AB002373 | Human mRNA for KIAA0375 gene, complete cds. |
| AB002375 | Human mRNA for KIAA0377 gene, complete cds. |
| AB002389 | Human mRNA for KIAA0391 gene, complete cds. |
| AB002804 | Homo sapiens mRNA for hSLK, complete cds. |
| AB003103 | Homo sapiens mRNA for 26S proteasome subunit p55, complete |
| | cds. |
| AB003698 | Homo sapiens mRNA for Cdc7-related kinase, complete cds. |
| AB005754 | Homo sapiens mRNA for LAK-1, complete cds. |
| AB006623 | Homo sapiens mRNA for KIAA0285 gene, complete cds. |
| AB006626 | Homo sapiens mRNA for KIAA0288 gene, complete cds. |
| AB006651 | Homo sapiens EXLM1 mRNA, complete cds. |
| AB007454 | Homo sapiens mRNA for chemokine LEC precursor, complete cds. |
| AB007860 | Homo sapiens KIAA0400 mRNA, complete cds. |
| AB007866 | Homo sapiens KIAA0406 mRNA, complete cds. |
| AB007870 | Homo sapiens KIAA0410 mRNA, complete cds. |
| AB007874 | Homo sapiens KIAA0414 mRNA, partial cds. |
| AB007879 | Homo sapiens KIAA0419 mRNA, complete cds. |
| AB007886 | Homo sapiens KIAA0426 mRNA, complete cds. |
| AB007927 | Homo sapiens mRNA for KIAA0458 protein, complete cds. |
| AB007939 | Homo sapiens mRNA for KIAA0470 protein, complete cds. |
| AB007940 | Homo sapiens mRNA for KIAA0471 protein, complete cds. |
| AB007942 | Homo sapiens mRNA for KIAA0473 protein, complete cds. |
| AB007944 | Homo sapiens mRNA for KIAA0475 protein, complete cds. |
| AB007945 | Homo sapiens mRNA for KIAA0476 protein, complete cds. |
| AB007949 | Homo sapiens mRNA for KIAA0480 protein, complete cds. |
| AB007950 | Homo sapiens mRNA for KIAA0481 protein, complete cds. |
| AB008226 | Homo sapiens FCMD mRNA for fukutin, complete cds. |
| AB011103 | Homo sapiens mRNA for KIAA0531 protein, complete cds. |
| AR011107 | Homo sapiens mRNA for KIAA0535 protein, complete cds. |
| AB011109 | Homo sapiens mRNA for KIAA0537 protein, complete cds. |
| AB011122 | Homo sapiens mRNA for KIAA0550 protein, complete cds. |
| AB011134 | Homo sapiens mRNA for KIAA0562 protein, complete cds. |
| AB011137 | Homo sapiens mRNA for KIAA0565 protein, complete cds. |
| AB011141 | Homo sapiens mRNA for KIAA0569 protein, complete cds. |
| AB011143 | Homo sapiens mRNA for KIAA0571 protein, complete cds. |
| AB011420 | Homo sapiens mRNA for DRAK1, complete cds. |
| AB012851 | Homo sapiens mRNA for Musashi, complete cds. |
| AB014517 | Homo sapiens mRNA for KIAA0617 protein, complete cds. |
| AB014526 | Homo sapiens mRNA for KIAA0626 protein, complete cds. |
| AB014528 | Homo sapiens mRNA for KIAA0628 protein, complete cds. |
| AB014551 | Homo sapiens mRNA for KIAA0651 protein, complete cds. |
| AB014552 | Homo sapiens mRNA for KIAA0652 protein, complete cds. |
| AB014560 | Homo sapiens mRNA for KIAA0660 protein, complete cds. |
| AB014569 | Homo sapiens mRNA for KIAA0669 protein, complete cds. |
| AB014585 | Homo sapiens mRNA for KIAA0685 protein, complete cds. |
| AB014588 | Homo sapiens mRNA for KIAA0688 protein, complete cds. |
| AB014598 | Homo sapiens mRNA for KIAA0698 protein, complete cds. |
| AB014605 | Homo sapiens mRNA for KIAA0705 protein, complete cds. |
| AB016193 | Homo sapiens Elk1 mRNA, complete cds. |
| AB016247 | Homo sapiens mRNA for sterol-C5-desaturase, complete cds. |
| AB016899 | Homo sapiens HGC6.1.1 mRNA, complete cds. |
| AB017642 | Homo sapiens mRNA for oxidative-stress responsive 1, complete |
| | cds. |
| AB017915 | Homo sapiens mRNA for condoroitin 6-sulfotransferase, complete |
| | cds. |
| AB018254 | Homo sapiens mRNA for KIAA0711 protein, complete cds. |
| AB018259 | Homo sapiens mRNA for KIAA0716 protein, complete cds. |
| AB018279 | Homo sapiens mRNA for KIAA0736 protein, complete cds. |
| AB018287 | Homo sapiens mRNA for KIAA0744 protein, complete cds. |
| AB018307 | Homo sapiens mRNA for KIAA0764 protein, complete cds. |
| AB018341 | Homo sapiens mRNA for KIAA0798 protein, complete cds. |
| AB018351 | Homo sapiens mRNA for KIAA0808 protein, complete cds. |
| AB018413 | Homo sapiens mRNA for Gab2, complete cds. |
| AB019517 | Homo sapiens PKIG mRNA for protein kinase inhibitor gamma, |
| | complete |
| AB020316 | Homo sapiens mRNA for dermatan/chondroitin sulfate |
| AB020639 | Homo sapiens mRNA for KIAA0832 protein, complete cds. |
| AB020642 | Homo sapiens mRNA for KIAA0835 protein, complete cds. |
| AB020651 | Homo sapiens mRNA for KIAA0844 protein, complete cds. |
| AB020655 | Homo sapiens mRNA for KIAA0848 protein, complete cds. |
| AB020659 | Homo sapiens mRNA for KIAA0852 protein, complete cds. |
| AB020686 | Homo sapiens mRNA for KIAA0879 protein, complete cds. |
| AB020700 | Homo sapiens mRNA for KIAA0893 protein, complete cds. |
| AB022663 | Homo sapiens HFB30 mRNA, complete cds. |
| AB023021 | Homo sapiens FUT9 mRNA for alpha-1,3-fucosyltransferase IX, |
| AB023141 | Homo sapiens mRNA for KIAA0924 protein, complete cds. |
| AB023153 | Homo sapiens mRNA for KIAA0936 protein, complete cds. |
| AB023155 | Homo sapiens mRNA for KIAA0938 protein, complete cds. |
| AB023158 | Homo sapiens mRNA for KIAA0941 protein, complete cds. |
| AB023169 | Homo sapiens mRNA for KIAA0952 protein, complete cds. |
| AB023172 | Homo sapiens mRNA for KIAA0955 protein, complete cds. |
| AB023183 | Homo sapiens mRNA for KIAA0966 protein, complete cds. |
| AB023187 | Homo sapiens mRNA for KIAA0970 protein, complete cds. |
| AB023188 | Homo sapiens mRNA for KIAA0971 protein, complete cds. |
| AB023207 | Homo sapiens mRNA for KIAA0990 protein, complete cds. |
| AB023214 | Homo sapiens mRNA for KIAA0997 protein, complete cds. |
| AB023225 | Homo sapiens mRNA for KIAA1008 protein, complete cds. |
| AB026118 | Homo sapiens mRNA for MALT1, complete cds. |
| AB026190 | Homo sapiens mRNA for Kelch motif containing protein, complete |
| | cds. |
| AB028964 | Homo sapiens mRNA for KIAA1041 protein, complete cds. |
| AB028965 | Homo sapiens mRNA for KIAA1042 protein, complete cds. |
| AB028967 | Homo sapiens mRNA for KIAA1044 protein, complete cds. |
| AB028996 | Homo sapiens mRNA for KIAA1073 protein, complete cds. |
| AB029024 | Homo sapiens mRNA for KIAA1101 protein, complete cds. |
| AB030653 | Homo sapiens mRNA for epsilon-adaptin, complete cds. |
| AF000145 | Homo sapiens germinal center kinase related protein kinase mRNA, |
| AF000367 | Homo sapiens cdc14 homolog mRNA, complete cds. |
| HSAF000982 | Homo sapiens dead box, X isoform (DBX) mRNA, alternative |
| | transcript |
| HSAF000984 | Homo sapiens dead box, Y isoform (DBY) mRNA, alternative |
| | transcript |
| HSAF000993 | Homo sapiens ubiquitous TPR motif, X isoform (UTX) mRNA, |
| AF001042 | Homo sapiens RNA editase (RED1) mRNA, complete cds. |
| AF001437 | Homo sapiens dihydrolipoamide dehydrogenase-binding protein |
| | mRNA, |
| AF001846 | Homo sapiens lymphoid phosphatase LyP1 mRNA, complete cds. |
| AF002697 | Homo sapiens E1B 19 K/Bcl-2-binding protein Nip3 mRNA, nuclear |
| | gene |
| AF003837 | Homo sapiens Jagged1 (JAG1) mRNA, complete cds. |
| AF004291 | Homo sapiens germ cell nuclear factor (GCNF) mRNA, complete |
| | cds. |
| AF004562 | Homo sapiens hUNC18a alternatively-spliced mRNA, complete |
| | cds. |
| AF004563 | Homo sapiens hUNC18b alternatively-spliced mRNA, complete |
| | cds. |
| AF004715 | Homo sapiens jerky gene product homolog mRNA, complete cds. |
| AF004841 | Homo sapiens CDO mRNA, complete cds. |
| AF005418 | Homo sapiens retinoic acid hydroxylase mRNA, complete cds. |
| AF006011 | Homo sapiens dishevelled 1 (DVL1) mRNA, complete cds. |
| AF006514 | Homo sapiens CHD2 mRNA, complete cds. |
| AF006621 | Homo sapiens embryonic lung protein (HUEL) mRNA, complete |
| | cds. |
| AF007111 | Homo sapiens MDM2-like p53-binding protein (MDMX) mRNA, |
| | complete |
| AF008915 | Homo sapiens EV15 homolog mRNA, complete cds. |
| AF009301 | Homo sapiens TEB4 protein mRNA, complete cds. |
| AF009801 | Homo sapiens homeodomain protein (BAPX1) mRNA, complete |
| | cds. |
| AF012072 | Homo sapiens eIF4GII mRNA, complete cds. |
| AF012126 | Homo sapiens zinc finger protein (ZNF198) mRNA, complete cds. |
| AF012535 | Homo sapiens death receptor 5 (DR5) mRNA, complete cds. |
| AF013168 | Homo sapiens hamartin (TSC1) mRNA, complete cds. |
| AF013970 | Homo sapiens MTG8-like protein MTGR1b mRNA, complete cds. |
| AF015592 | Homo sapiens Cdc7 (CDC7) mRNA, complete cds. |
| AF016005 | Homo sapiens chromosome 1 atrophin-1 related protein (DRPLA) |
| | mRNA, |
| AF016266 | Homo sapiens TRAIL receptor 2 mRNA, complete cds. |
| AF016268 | Homo sapiens death receptor 5 (DR5) mRNA, complete cds. |
| AF016833 | Homo sapiens maltase-glucoamylase mRNA, complete cds. |
| AF016849 | Homo sapiens apoptosis inducing receptor TRAIL-R2 (TRAILR2) |
| | mRNA, |
| AF019047 | Homo sapiens receptor activator of nuclear factor kappa B ligand |
| AF019386 | Homo sapiens heparan sulfate 3-O-sulfotransferase-1 precursor |
| AF019770 | Homo sapiens macrophage inhibitory cytokine-1 (MIC-1) mRNA, |
| AF020089 | Homo sapiens PEN11B mRNA, complete cds. |
| AF021336 | Homo sapiens DNA damage-inducible RNA binding protein |
| | (A18hnRNP) |
| AF022375 | Homo sapiens vascular endothelial growth factor mRNA, complete |
| | cds. |
| AF022654 | Homo sapiens homeodomain protein (OG12) mRNA, complete cds. |
| AF023456 | Homo sapiens protein phosphatase with EF-hands-2 long form |
| | (PPEF-2) |
| AF025654 | Homo sapiens mRNA capping enzyme (HCE) mRNA, complete |
| | cds. |
| AF026245 | Homo sapiens yotiao mRNA, complete cds. |
| AF027706 | Homo sapiens serine/threonine kinase RICK (RICK) mRNA, |
| | complete |
| AF028593 | Homo sapiens transmembrane protein Jagged 1 (HJ1) mRNA, |
| | complete |
| AF029729 | Homo sapiens neuralized mRNA, complete cds. |
| AF030186 | Homo sapiens glypican-4 (GPC4) mRNA, complete cds. |
| AF030409 | Homo sapiens sodium-hydrogen exchanger 6 (NHE-6) mRNA, |
| | nuclear gene |
| AF030455 | Homo sapiens epithelial V-like antigen precursor (EVA) mRNA, |
| AF030555 | Homo sapiens acyl-CoA synthetase 4 (ACS4) mRNA, complete cds. |
| AF030880 | Homo sapiens pendrin (PDS) mRNA, complete cds. |
| AF031167 | Homo sapiens interleukin 15 precursor (IL-15) mRNA, complete |
| | cds. |
| AF032885 | Homo sapiens forkhead protein (FKHR) mRNA, complete cds. |
| AF035013 | Homo sapiens cell cycle related kinase mRNA, complete cds. |
| AF035582 | Homo sapiens CASK mRNA, complete cds. |
| AF036718 | Homo sapiens FGFR signalling adaptor SNT-2 mRNA, complete |
| | cds. |
| AF038392 | Homo sapiens pre-mRNA splicing factor (PRP17) mRNA, complete |
| | cds. |
| AF038563 | Homo sapiens atrophin-1 interacting protein 1 (AIP1) mRNA, |
| | complete |
| AF039067 | Homo sapiens anti-death protein (IEX-1L) mRNA, complete cds. |
| AF039747 | Homo sapiens cadherin-10 (CDH10) mRNA, complete cds. |
| AF040701 | Homo sapiens TATA binding protein associated factor (TAFII150) |
| AF042378 | Homo sapiens spindle pole body protein spc98 homolog GCP3 |
| | mRNA, |
| AF042729 | Homo sapiens lithium-sensitive myo-inositol monophosphatase A1 |
| AF043976 | Homo sapiens CLCA homolog (hCLCA3) mRNA, complete cds. |
| AF044221 | Homo sapiens HCG-1 protein (HCG-1) mRNA, complete cds. |
| AF044588 | Homo sapiens protein regulating cytokinesis 1 (PRC1) mRNA, |
| | complete |
| AF045451 | Homo sapiens transcriptional regulatory protein p54 mRNA, |
| | complete |
| AF046059 | Homo sapiens cytokine receptor related protein 4 (CYTOR4) |
| | mRNA, |
| AF047033 | Homo sapiens sodium bicarbonate cotransporter 3 (SLC4A7) |
| | mRNA, |
| AF047440 | Homo sapiens ribosomal protein L33-like protein mRNA, complete |
| | cds. |
| AF047472 | Homo sapiens spleen mitotic checkpoint BUB3 (BUB3) mRNA, |
| | complete |
| AF048731 | Homo sapiens cyclin T2a mRNA, complete cds. |
| AF049140 | Homo sapiens MMS2 (MMS2) mRNA, complete cds. |
| AF049910 | Homo sapiens TACC1 (TACC1) mRNA, complete cds. |
| AF051323 | Homo sapiens Src-associated adaptor protein (SAPS) mRNA, |
| | complete |
| AF051850 | Homo sapiens supervillin mRNA, complete cds. |
| AF051894 | Homo sapiens 15 kDa selenoprotein mRNA, complete cds. |
| AF052224 | Homo sapiens neuronal double zinc finger protein (ZNF231) |
| | mRNA, |
| AF053304 | Homo sapiens mitotic checkpoint component Bub3 (BUB3) mRNA, |
| AF053712 | Homo sapiens osteoprotegerin ligand mRNA, complete cds. |
| AF054176 | Homo sapiens angiotensin/vasopressin receptor AII/AVP mRNA, |
| AF055013 | Homo sapiens clone 24695 guanine nucleotide-binding protein |
| | alpha-i |
| AF055467 | Homo sapiens monotactin-1 mRNA, complete cds. |
| AF055636 | Homo sapiens leucine-rich glioma-inactivated protein precursor |
| AF056032 | Homo sapiens kynurenine 3-hydroxylase mRNA, complete cds. |
| AF056320 | Homo sapiens inducible 6-phosphofructo-2-kinase/fructose |
| AF056929 | Homo sapiens sarcosin mRNA, complete cds. |
| AF058291 | Homo sapiens estrogen-related receptor gamma mRNA, complete |
| | cds. |
| AF059569 | Homo sapiens actin binding protein MAYVEN mRNA, complete |
| | cds. |
| AF059611 | Homo sapiens nuclear matrix protein NRP/B (NRPB) mRNA, |
| | complete |
| AF059617 | Homo sapiens serum-inducible kinase mRNA, complete cds. |
| AF060877 | Homo sapiens Gz-selective GTPase-activating protein (ZGAP1) |
| | mRNA, |
| AF061016 | Homo sapiens UDP-glucose dehydrogenase (UGDH) mRNA, |
| | complete cds. |
| AF061326 | Homo sapiens T41p (C8orf1) mRNA, complete cds. |
| AF061573 | Homo sapiens protocadherin (PCDH8) mRNA, complete cds. |
| AF061936 | Homo sapiens diacylglycerol kinase iota (DGKi) mRNA, complete |
| | cds. |
| AF063301 | Homo sapiens keratan sulfate proteoglycan mRNA, complete cds. |
| AF063605 | Homo sapiens brain my047 protein mRNA, complete cds. |
| AF064244 | Homo sapiens intersectin long form mRNA, complete cds. |
| AF064548 | Homo sapiens low-density lipoprotein receptor-related protein 5 |
| AF064607 | Homo sapiens GC20 protein mRNA, complete cds. |
| AF067170 | Homo sapiens alpha endosulfine mRNA, complete cds. |
| AF068006 | Homo sapiens haemopoietic progenitor homeobox HPX42B |
| | (HPX42B) mRNA, |
| AF068302 | Homo sapiens choline/ethanolaminephosphotransferase (CEPT1) |
| | mRNA, |
| AF068836 | Homo sapiens cytohesin binding protein HE mRNA, complete cds. |
| AF069313 | Homo sapiens WSB-1 mRNA, complete cds. |
| AF069747 | Homo sapiens MTG8-like protein MTGR1a mRNA, complete cds. |
| AF070674 | Homo sapiens inhibitor of apoptosis protein-1 (MIHC) mRNA, |
| | complete |
| AF071309 | Homo sapiens OPA-containing protein mRNA, complete cds. |
| AF071594 | Homo sapiens MMSET type I (MMSET) mRNA, complete cds. |
| AF073310 | Homo sapiens insulin receptor substrate-2 (IRS2) mRNA, complete |
| AF073518 | Homo sapiens small EDRK-rich factor 1, short isoform (SERF1) |
| | mRNA, |
| AF073519 | Homo sapiens small EDRK-rich factor 1, long isoform (SERF1) |
| | mRNA, |
| AF073958 | Homo sapiens cytokine-inducible SH2 protein 6 (CISH6) mRNA, |
| AF076844 | Homo sapiens Hus1-like protein (HUS1) mRNA, complete cds. |
| AF077036 | Homo sapiens HSPC012 mRNA, complete cds. |
| AF077041 | Homo sapiens SIH002 mRNA, complete cds. |
| AF077052 | Homo sapiens protein translation factor sui1 homolog mRNA, |
| | complete |
| AF077205 | Homo sapiens HSPC019 mRNA, complete cds. |
| AF077599 | Homo sapiens hypothetical SBBI03 protein mRNA, complete cds. |
| AF077820 | Homo sapiens LDL receptor member LR3 mRNA, complete cds. |
| AF078165 | Homo sapiens conductin mRNA, complete cds. |
| AF079566 | Homo sapiens ubiquitin-like protein activating enzyme (UBA2) |
| | mRNA, |
| AF081259 | Homo sapiens testis-specific chromodomain Y-like protein (CDYL) |
| AF083106 | Homo sapiens sirtuin type 1 (SIRT1) mRNA, complete cds. |
| AF083217 | Homo sapiens WD repeat protein WDR3 (WDR3) mRNA, |
| | complete cds. |
| AF084530 | Homo sapiens cyclin-D binding Myb-like protein mRNA, complete |
| | cds. |
| AF089744 | Homo sapiens xenotropic and polytropic murine leukemia virus |
| AF090384 | Homo sapiens SUMO-1-activating enzyme E1 C subunit (UBA2) |
| | mRNA, |
| AF091083 | Homo sapiens clone 628 unknown mRNA, complete sequence. |
| AF092051 | Homo sapiens beta-1,3-N-acetylglucosaminyltransferase mRNA, |
| AF093774 | Homo sapiens type 2 iodothyronine deiodinase mRNA, complete |
| | cds |
| AF097159 | Homo sapiens UDP-Gal: glucosylceramide |
| AF099989 | Homo sapiens Ste-20 related kinase SPAK mRNA, complete cds. |
| AF100740 | Homo sapiens ARF-family of Ras related GTPases mRNA, |
| | complete cds. |
| AF100779 | Homo sapiens connective tissue growth factor related protein WISP-1 |
| AF101441 | Homo sapiens bone morphogenetic protein 10 (BMP10) mRNA, |
| | complete |
| AF103796 | Homo sapiens placenta-specific ATP-binding cassette transporter |
| AF104032 | Homo sapiens L-type amino acid transporter subunit LAT1 mRNA, |
| AF104419 | Homo sapiens decoy receptor 3 (DcR3) mRNA, complete cds. |
| AF105365 | Homo sapiens K-Cl cotransporter KCC4 mRNA, complete cds. |
| AF105377 | Homo sapiens heparan sulfate D-glucosaminyl 3-O- |
| | sulfotransferase-3B |
| AF106622 | Homo sapiens mitochondrial inner membrane preprotein translocase |
| AF106683 | Homo sapiens WSB-1 mRNA, complete cds. |
| AF106684 | Homo sapiens WSB-1 isoform mRNA, complete cds. |
| AF109126 | Homo sapiens stromal cell-derived receptor-1 beta mRNA, |
| | complete |
| AF109219 | Homo sapiens Mcd4p homolog mRNA, complete cds. |
| AF109735 | Homo sapiens ubiquitous 6-phosphofructo-2-kinase/fructose |
| AF110146 | Homo sapiens eukaryotic translation initiation factor 2 alpha |
| AF110400 | Homo sapiens fibroblast growth factor 19 (FGF19) mRNA, |
| | complete |
| AF112227 | Homo sapiens TDE homolog mRNA, complete cds. |
| AF112299 | Homo sapiens integral inner nuclear membrane protein MAN1 |
| | mRNA, |
| AF114263 | Homo sapiens clone HH114 unknown mRNA. |
| AF116846 | Homo sapiens bright and dead ringer gene product homologous |
| | protein |
| AF117210 | Homo sapiens host cell factor 2 (HCF-2) mRNA, complete cds. |
| AF117754 | Homo sapiens thyroid hormone receptor-associated protein complex |
| AF120151 | Homo sapiens cytokine receptor-like molecule 9 (CREME9) |
| | mRNA, |
| AF121951 | Homo sapiens CAAX prenyl protein protease RCE1 (RCE1) |
| | mRNA, |
| AF123094 | Homo sapiens API2-MLT fusion protein (API2-MLT) mRNA, |
| | complete cds. |
| AF124250 | Homo sapiens SH2-containing protein Nsp2 mRNA, complete cds. |
| AF125042 | Homo sapiens bisphosphate 3′-nucleotidase mRNA, complete cds. |
| AF125101 | Homo sapiens HSPC040 protein mRNA, complete cds. |
| AF130356 | Homo sapiens MALT lymphoma associated translocation (MLT) |
| | mRNA, |
| AF132297 | Homo sapiens cytokine-inducible SH2-containing protein (G18) |
| | mRNA, |
| AF132944 | Homo sapiens CGI-10 protein mRNA, complete cds. |
| AF132960 | Homo sapiens CGI-26 protein mRNA, complete cds. |
| AF132968 | Homo sapiens CGI-34 protein mRNA, complete cds. |
| AF133820 | Homo sapiens titin-like protein (TTID) mRNA, complete cds. |
| AF133845 | Homo sapiens corin mRNA, complete cds. |
| AF134802 | Homo sapiens cofilin isoform 1 mRNA, complete cds. |
| AF134803 | Homo sapiens cofilin isoform 2 mRNA, complete cds. |
| AF135794 | Homo sapiens AKT3 protein kinase mRNA, complete cds. |
| AF139658 | Homo sapiens origin recognition complex subunit 6 (ORC6) |
| | mRNA, |
| AF148213 | Homo sapiens aggrecanase-1 mRNA, complete cds. |
| AF151522 | Homo sapiens hairy and enhancer of split related-1 (HESR-1) |
| | mRNA, |
| AF151837 | Homo sapiens CGI-79 protein mRNA, complete cds. |
| AF151865 | Homo sapiens CGI-107 protein mRNA, complete cds. |
| AF151869 | Homo sapiens CGI-111 protein mRNA, complete cds. |
| AF151881 | Homo sapiens CGI-123 protein mRNA, complete cds. |
| AF151899 | Homo sapiens CGI-141 protein mRNA, complete cds. |
| AF151900 | Homo sapiens CGI-142 protein mRNA, complete cds. |
| AF151903 | Homo sapiens CGI-145 protein mRNA, complete cds. |
| AF151906 | Homo sapiens CGI-148 protein mRNA, complete cds. |
| AF153330 | Homo sapiens thiamine carrier 1 (TC1) mRNA, complete cds. |
| AF158555 | Homo sapiens glutaminase C mRNA, complete cds. |
| AF165522 | Homo sapiens ras-related GTP-binding protein 4b (RAB4B) |
| | mRNA, |
| HSJ001388 | Homo Sapiens, RP58 cDNA for complete mRNA. |
| HSA5821 | Homo sapiens mRNA for X-like 1 protein. |
| HSAJ6470 | Homo sapiens mRNA for cartilage-associated protein (CASP). |
| HS010046 | Homo sapiens mRNA for Rho guanine nucleotide-exchange factor, |
| HSA011785 | Homo sapiens mRNA for Six9 protein. |
| HSA012370 | Homo sapiens mRNA for NAALADase II protein. |
| HSA012755 | Homo sapiens mRNA for TL132. |
| HSA132545 | Homo sapiens mRNA for protein kinase. |
| HSAJ4741 | Homo sapiens mRNA for matrilin-3. |
| HSAJ4901 | Homo sapiens mRNA for ZNF198 protein. |
| HSA238243 | Homo sapiens mRNA for phospholipase A2 activating protein. |
| HSA238248 | Homo sapiens mRNA for centaurin beta2. |
| HSA238701 | Homo sapiens mRNA for alpha-3-fucosyltransferase. |
| HS1039K5A | Novel human mRNA similar to mouse gene PICK1 (TR: Q62083). |
| HSM800381 | Homo sapiens mRNA; cDNA DKFZp566D213 (from clone |
| | DKFZp566D213). |
| HSM800571 | Homo sapiens mRNA; cDNA DKFZp564M112 (from clone |
| | DKFZp564M112). |
| HSM800697 | Homo sapiens mRNA; cDNA DKFZp434K151 (from clone |
| | DKFZp434K151). |
| HSM800724 | Homo sapiens mRNA; cDNA DKFZp434F122 (from clone |
| | DKFZp434F122). |
| HUMPRSI | Homo sapiens mRNA for phosphoribosyl pyrophosphate synthetase |
| HUM2OGDH | Human mRNA for 2-oxoglutarate dehydrogenase, complete cds. |
| HUMHM63 | Human mRNA for FMLP-related receptor (HM63). |
| HUMHM89 | Human mRNA for HM89. |
| HUMTF147 | Human mRNA for transcription factor, E4TF1-47, complete cds. |
| HUMRSC192 | Human mRNA for KIAA0003 gene, complete cds. |
| HUMRSC1083 | Human mRNA for KIAA0010 gene, complete cds. |
| D13641 | Human mRNA for KIAA0016 gene, complete cds. |
| HUMLYK | Homo sapiens mRNA for ITK, complete cds. |
| HUMID2HC | Human mRNA for Id-2H, complete cds. |
| HUMMGC24 | Human mRNA for MGC-24, complete cds. |
| HUMAREB6 | Human mRNA for transcription factor AREB6, complete cds. |
| HUM6PTS1 | Human mRNA for 6-pyruvoyl-tetrahydropterin synthase, complete |
| | cds. |
| HUMPTPB1 | Human mRNA for protein tyrosine phosphatase (PTP-BAS, type 1), |
| HUMPTPB2 | Human mRNA for protein tyrosine phosphatase (PTP-BAS, type 2), |
| HUMPTPB3 | Human mRNA for protein tyrosine phosphatase (PTP-BAS, type 3), |
| HUMORFKA | Human mRNA for KIAA0032 gene, complete cds. |
| HUMORFR | Human mRNA for KIAA0040 gene, complete cds. |
| HUMHGLUT1 | Human mRNA for glutamate transporter, complete cds. |
| HUMPTKA | Human mRNA for Tec protein-tyrosine kinase, complete cds. |
| D30783 | Homo sapiens mRNA for epiregulin, complete cds. |
| HUMDRPLA1 | Human DRPLA mRNA for ORF, complete cds. |
| HUMORFKG1L | Human mRNA for KIAA0059 gene, complete cds. |
| HUMKIAAB | Human mRNA for KIAA0087 gene, complete cds. |
| HUMPLCE | Human mRNA for phospholipase C, complete cds. |
| HUMSCM1A | Human mRNA for SCM-1 (single cysteine motif-1), complete cds. |
| HUMUPST2 | Human apM1 mRNA for GS3109 (novel adipose specific collagen- |
| | like) |
| HUMEOTAXIN | Human mRNA for eotaxin, complete cds. |
| HUMNAK1 | Human NAK1 mRNA for DNA binding protein, complete cds. |
| HUMNRAMP1A | Human mRNA for NRAMP1, complete cds. |
| HUMHRH1 | Human mRNA for RNA helicase (HRH1), complete cds. |
| D50678 | Human mRNA for apolipoprotein E receptor 2, complete cds. |
| HUMPLAA | Homo sapiens mRNA for placental leucine aminopeptidase, |
| | complete |
| HUMCGA | Homo sapiens mRNA for ceramide glucosyltransferase, complete |
| | cds. |
| D50917 | Human mRNA for KIAA0127 gene, complete cds. |
| D50931 | Human mRNA for KIAA0141 gene, complete cds. |
| D63476 | Human mRNA for KIAA0142 gene, complete cds. |
| D64015 | Homo sapiens mRNA for T-cluster binding protein, complete cds. |
| HUMCIRPA | Homo sapiens mRNA for CIRP, complete cds. |
| D78579 | Homo sapiens mRNA for neuron derived orphan receptor, complete |
| | cds. |
| D79993 | Human mRNA for KIAA0171 gene, complete cds. |
| D82347 | Homo sapiens mRNA for NeuroD, complete cds. |
| D83017 | Homo sapiens mRNA for nel-related protein, complete cds. |
| D83175 | Homo sapiens WNT7a mRNA, complete cds. |
| D83197 | Homo sapiens mRNA for ankyrin repeat protein, complete cds. |
| D84145 | Human WS-3 mRNA, complete cds. |
| D84276 | Homo sapiens mRNA for CD38, complete cds. |
| HUMHAS | Homo sapiens mRNA for hyaluronan synthase, complete cds. |
| D84454 | Human mRNA for UDP-galactose translocator, complete cds. |
| D85181 | Homo sapiens mRNA for fungal sterol-C5-desaturase homolog, |
| | complete |
| D86958 | Human mRNA for KIAA0203 gene, complete cds. |
| D86959 | Human mRNA for KIAA0204 gene, complete cds. |
| D86967 | Human mRNA for KIAA0212 gene, complete cds. |
| D86979 | Human mRNA for KIAA0226 gene, complete cds. |
| D86985 | Human mRNA for KIAA0232 gene, complete cds. |
| D87328 | Homo sapiens mRNA for HCS, complete cds. |
| D87434 | Human mRNA for KIAA0247 gene, complete cds. |
| D87445 | Human mRNA for KIAA0256 gene, complete cds. |
| D87447 | Human mRNA for KIAA0258 gene, complete cds. |
| D87461 | Human mRNA for KIAA0271 gene, complete cds. |
| D87467 | Human mRNA for KIAA0277 gene, complete cds. |
| D87717 | Human mRNA for KIAA0013 gene, complete cds. |
| D88153 | Homo sapiens mRNA for HYA22, complete cds. |
| HUMAPR | Human ATL-derived PMA-responsive (APR) peptide mRNA. |
| HUMLCA1 | Human mRNA for 1-caldesmon I. |
| HUMLCA2 | Human mRNA for 1-caldesmon II. |
| E00044 | DNA coding of LeIF B. |
| E00047 | DNA coding of LeIF E. |
| E00048 | DNA coding of LeIF F. |
| E00051 | DNA coding of LeIF I. |
| E00052 | DNA coding of LeIF J. |
| E00095 | DNA coding of interferon-gamma. |
| E00102 | DNA coding of interferon(LyIFN-alpha-1). |
| E00104 | DNA coding of interferon(LyIFN-alpha-2). |
| E00124 | DNA coding of alpha-interferon Gx-1. |
| E00173 | cDNA encoding human interferon-8′(1). |
| E00176 | cDNA encoding human interferon-alpha-3. |
| E00294 | cDNA encoding human interferon. |
| E00372 | cDNA encoding human interleukin-2. |
| E00380 | DNA coding for human interferon gamma. |
| E01058 | cDNA encoding human interleukin-1 precursor. |
| E01219 | cDNA encoding human G-CSF. |
| E01275 | cDNA encoding human lymphotoxin polypeptide. |
| E01467 | DNA encoding human prourokinase. |
| E01483 | cDNA encoding T cell replacing factor. |
| E01537 | DNA encoding human B-cell differentiation factor. |
| E01804 | cDNA encoding human polypeptide having lymphotoxin activity. |
| E02167 | cDNA encoding human TL-4. |
| E03588 | DNA encoding human NGF-like peptide. |
| E07650 | cDNA encoding endothelin receptor, ETB-receptor. |
| E07862 | DNA encoding the pro region, NGF2/NT-3 and its vicinity. |
| HUMSRF | Human serum response factor (SRF) mRNA, complete cds. |
| HUMGRO | Human gro (growth regulated) gene. |
| HUMPTHL | Human, parathyroid-like protein (associated with humoral) |
| HUMCALLA | Human common acute lymphoblastic leukemia antigen (CALLA) |
| | mRNA, |
| HUMPTHLHA | Human renal carcinoma parathgrad hormone-like peptide mRNA, |
| HUMALPHLA | Human phosphatase 2A mRNA, complete cds. |
| HUMLGTPA | Human liver glucose transporter-like protein (GLUT2), complete |
| | cds. |
| HUMACT2A | Human activation (Act-2) mRNA, complete cds. |
| HUMGFB | Human basic fibroblast growth factor (bFGF) 22.5 kd, 21 kd and 18 kd |
| HUMET3 | Human endothelin 3 (EDN3) mRNA, complete cds. |
| HUMIL1 | Human monocyte interleukin 1 (IL-1) mRNA, complete cds. |
| HUMCALREC | Human calcitonin receptor mRNA, complete cds. |
| HUMBASONU | Human zinc finger protein basonuclin mRNA, complete cds. |
| HUMGUABIND | Human nucleotide binding protein mRNA, complete cds. |
| HUMERCC6A | Human excision repair protein ERCC6 mRNA, complete cds. |
| HUMATPCU | Human putative Cu++-transporting P-type ATPase mRNA, |
| | complete cds. |
| HUMTRANSCR | Human transcription factor mRNA, complete cds. |
| HUMGPCR | Human (clone L5) orphan G protein-coupled receptor mRNA, |
| | complete |
| HUMCD40L | Human CD40-ligand mRNA, complete cds. |
| HUMTGFB3C | Human transforming growth factor-beta type III receptor (TGF- |
| | beta) |
| HUMTKTCS | Homo sapiens T cell-specific tyrosine kinase mRNA, complete cds. |
| HUMCELGROR | Human cellular growth-regulating protein mRNA, complete cds. |
| HUMPDE7A | Homo sapiens cAMP phosphodiesterase PDE7 (PDE7A1) mRNA, |
| | complete |
| HUMTHRSPO | Human thrombospondin 2 (THBS2) mRNA, complete cds. |
| HUMTR3A | Human TR3 orphan receptor mRNA, complete cds. |
| HUMAF4Y | Human AF-4 mRNA, complete cds. |
| HUMMHCREP | Human MHC class I-related protein mRNA, complete cds. |
| HUMENDOSYN | Human endoperoxide synthase type II mRNA, complete cds. |
| HUMGAD67X | Human glutamate decarboxylase (GAD67) mRNA, complete cds. |
| HUMAHREC | Human AH-receptor mRNA, complete cds. |
| HUMOCTF1A | Human octamer binding transcription factor 1 (OTF1) mRNA, |
| | complete |
| HUMIPLAS | Human I-plastin mRNA, complete cds. |
| HUMWNT5A | Homo sapiens proto-oncogene (Wnt-5a) mRNA, complete cds. |
| HUMPDEG | Human phosphodiesterase mRNA, complete cds. |
| HUMATP2B2X | Human plasma membrane calcium ATPase isoform 2 (ATP2B2) |
| | mRNA, |
| HUMGALC | Homo sapiens galactocerebrosidase (GALC) mRNA, complete cds. |
| HUMPWD | Homo sapiens (PWD) gene mRNA, 3′ end. |
| HUMPBXPROA | Homo sapiens paired box protein mRNA, complete cds. |
| HUMX104A | Human X104 mRNA, complete cds. |
| HUMB2CHIM | Homo sapiens beta2-chimaerin mRNA, complete cds. |
| HUMIQGA | Homo sapiens ras GTPase-activating-like protein (IQGAP1) |
| | mRNA, |
| HUMRPTK | Homo sapiens receptor protein-tyrosine kinase (HEK11) mRNA, |
| HUMGT198A | Homo sapiens GT198 mRNA, complete ORF. |
| HUMTFSL1B | Homo sapiens transcription factor SL1 mRNA, complete cds. |
| HUMSCPB | Homo sapiens TNFR2-TRAF signalling complex protein mRNA, |
| | complete |
| HUMCOX17R | Homo sapiens COX17 mRNA, complete cds. |
| HUMPTPC | Human protein tyrosine phosphatase mRNA, complete cds. |
| HUMKI32R | Homo sapiens inwardly rectifying potassium channel (Kir3.2) |
| | mRNA, |
| HUMTNFAA | Human tumor necrosis factor (TNF) mRNA. |
| HUMIFNAIP | Human interferon-alpha type I' mRNA, complete cds. |
| HUMGMCSFA | Human granulocyte-macrophage colony stimulating factor (GM- |
| | CSF) |
| HUMSISPDG | Human c-sis/platelet-derived growth factor 2 (SIS/PDGF2) mRNA, |
| HUMBCL2A | Human B-cell leukemia/lymphoma 2 (bcl-2) proto-oncogene mRNA |
| HUMVTNR | Human cell adhesion protein (vitronectin) receptor alpha subunit |
| HUMBCL2C | Human bcl-2 mRNA. |
| HUMIL1BA | Human interleukin 1-beta (IL1B) mRNA, complete cds. |
| HUMIF4E | Homo sapiens cap-binding protein mRNA, complete cds. |
| HUMUKPM | Human pro-urokinase mRNA, complete cds. |
| HUMLPL | Human lipoprotein lipase mRNA, complete cds. |
| HUMCYES1 | Human c-yes-1 mRNA. |
| HUMSTS | Human steroid sulfatase (STS) mRNA, complete cds. |
| HUMTHM | Human endothelial cell thrombomodulin mRNA, complete cds. |
| HUMPIM1 | Human pim-1 oncogene mRNA, complete cds. |
| HUMMCR | Human mineralocorticoid receptor mRNA (hMR), complete cds. |
| HUMHOXB | Human homeo box c8 protein, mRNA, complete cds. |
| HUMPTHRP | Human parathyroid hormone-related protein mRNA, complete cds. |
| HUMPAI2B | Human plasminogen activator inhibitor 2 (PAI-2) mRNA, complete |
| | cds. |
| HUMTGFB2A | Human transforming growth factor-beta-2 mRNA, complete cds. |
| HUMTRO | Human tropomyosin mRNA, complete cds. |
| HUMIL3A | Human interleukin 3 (IL-3) mRNA, complete cds, clone pcD-SR- |
| | alpha. |
| HUMGTLPA | Human glucose transporter-like protein-III (GLUT3), complete cds. |
| HUMGFIBP | Human insulin-like growth factor (IGF) binding protein mRNA, |
| HUMSRTR2A | Human steroid receptor TR2 mRNA, complete cds. |
| HUMENOG | Human neuron-specific gamma-2 enolase, complete cds. |
| HUMSTCPC | Homo sapiens secreted T cell protein (H400; SIS-gamma) mRNA, |
| HUMTHYP | Human parathymosin mRNA, complete cds. |
| HUMEAR1A | Human triiodothyronine recptor (THRA1, ear1) mRNA, complete |
| | cds. |
| HUMELK1A | Homo sapiens tyrosine kinase (ELK1) oncogene mRNA, complete |
| | cds. |
| HUMNATPEP | Human natriuretic peptide precursor mRNA, complete cds. |
| HUMCYTNEWA | Homo sapiens (clone pAT 464) potential lymphokine/cytokine |
| | mRNA, |
| HUMCYTNEWB | Homo sapiens (clone pAT 744) potential lymphokine/cytokine |
| | mRNA, |
| HUMPFKM23 | Human muscle phosphofructokinase (PFKM) mRNA, complete cds. |
| HUMMONAP | Human monocyte-derived neutrophil-activating protein (MONAP) |
| | mRNA, |
| HUME2B | Homo sapiens nuclear-encoded mitochondrial branched chain |
| HUMIFNAM1 | Human interferon (IFN-alpha-M1) mRNA, complete cds. |
| HUMIL1RA | Human interleukin 1 receptor mRNA, complete cds. |
| HUMCREB | Human transactivator protein (CREB) mRNA, complete cds. |
| HUMIFNN | Human leukocyte interferon-alpha mRNA, complete cds, clone |
| | pIFN105. |
| HUMIFNB1 | Human interferon beta-1 (IFN-beta-1) mRNA, complete cds. |
| HUMCFTRM | Human cystic fibrosis mRNA, encoding a presumed transmembrane |
| HUMRNPB1A | Human hnRNP B1 protein mRNA. |
| HUMRNPA2A | Human hnRNP A2 protein mRNA. |
| HUMCNRA1 | Human calcineurin A1 mRNA, complete cds. |
| HUMCNRAB | Human calcineurin A2 mRNA, complete cds. |
| HUMZFX | Human zinc finger protein X-linked (ZFX) mRNA, complete cds. |
| HUMELAMA1A | Human endothelial leukocyte adhesion molecule I (ELAM1) |
| | mRNA, |
| HUMARXC | Human amphiregulin (AR) mRNA, complete cds, clones lambda- |
| | AR1 |
| HUMCNR | Human calcineurin B mRNA, complete cds. |
| HUMGFIBPL | Human insulin-like growth factor binding protein mRNA, complete |
| HUMTSG6A | Human tumor necrosis factor-inducible (TSG-6) mRNA fragment, |
| HUMPTCAA | Human papillary thyroid carcinoma-encoded protein mRNA, |
| | complete |
| HUMDAFB | Human decay-accelerating factor mRNA, complete cds. |
| HUMSRICPA | Human sorcin CP-22 mRNA, complete cds. |
| HUMHBLOD | Human GDP-L-fucose: beta-D-galactoside 2-alpha-1- |
| | fucosyltransferase |
| HUMGROB5 | Human cytokine (GRO-beta) mRNA, complete cds. |
| HUMGROG5 | Human cytokine (GRO-gamma) mRNA, complete cds. |
| HUMCSDF1 | Human macrophage-specific colony-stimulating factor (CSF-1) |
| | mRNA, |
| HUMP58GTA | Human p58/GTA (galactosyltransferase associated protein kinase) |
| HUMMEMGL1 | Human MEM-102 glycoprotein mRNA, complete cds. |
| HUMRARGA | Human retinoic acid receptor gamma 1 mRNA, complete cds. |
| HUMINTAZ | Human interferon-alpha mRNA, complete cds. |
| HUMIL6CSF | Human interleukin 6 mRNA, complete cds. |
| HUMPIM1LE | Human h-pim-1 protein (h-pim-1) mRNA, complete cds. |
| HUMKRASM | Human K-ras oncogene protein mRNA, complete cds. |
| HUMSYTA | Human synaptotagmin mRNA, complete cds. |
| HUMIL10 | Human interleukin 10 (IL10) mRNA, complete cds. |
| HUMGROB | Human gro-beta mRNA, complete cds. |
| HUMIDSX | Human iduronate 2-sulfatase mRNA, complete cds. |
| HUMELFTL | Human ELAM-1 ligand fucosyltransferase (ELFT) mRNA, |
| | complete cds. |
| HUMECK | Human protein tyrosine kinase mRNA, complete cds. |
| HUMA20 | Human tumor necrosis factor alpha inducible protein A20 mRNA, |
| HUMCD48 | Human pan-leukocyte antigen (CD48) mRNA, complete cds. |
| HUMHBEGF | Human heparin-binding EGF-like growth factor mRNA, complete |
| | cds. |
| HUMTGFBC | Human transforming growth factor-beta (tgf-beta) mRNA, complete |
| HUMKGF | Human keratinocyte growth factor mRNA, complete cds. |
| HUMCS1PA | Human cleavage signal 1 protein mRNA, complete cds. |
| HUMGABAR | Human gamma-aminobutyric acid receptor type A rho-1 subunit |
| | (GABA-A) |
| HUMLHHCGR | Human luteinizing hormone-choriogonadrotropin receptor mRNA, |
| HUMRACPC | Human rac protein kinase alpha mRNA, complete cds. |
| HUMPHLAM | Human phospholamban mRNA, complete cds. |
| HUMCALD | Human caldesmon mRNA, complete cds. |
| HUMET2A | Human endothelin 2 (ET2) mRNA, complete cds. |
| HUMNKSFP40 | Human natural killer cell stimulatory factor (NKSF) mRNA, |
| | complete |
| HUMNKSFP35 | Human natural killer cell stimulatory factor (NKSF) mRNA, |
| | complete |
| HUMCDC2A | Human cdc2-related protein kinase mRNA, complete cds. |
| HUMPLA2 | Homo sapiens phosphatidylcholine 2-acylhydrolase (cPLA2) |
| | mRNA, |
| HUMGATA | Human GATA-binding protein (GATA2) mRNA, complete cds. |
| HUMMAD3A | Homo sapiens MAD-3 mRNA encoding IkB-like activity, complete |
| | cds. |
| HUMLHCGR | Homo sapiens lutropin/choriogonadotropin receptor (LHCGR) |
| | mRNA, |
| HUMHKATPB | Human H, K-ATPase beta subunit mRNA, complete cds. |
| HUMGATA2A | Human transcription factor GATA-2 (GATA-2) mRNA, complete |
| | cds. |
| HUME16GEN | Human E16 mRNA, complete cds. |
| HUMHEB | Human HEB helix-loop-helix protein (HEB) mRNA, complete cds. |
| HUMGAD67A | Human glutamate decarboxylase (GAD67) mRNA, complete cds. |
| HUMCDSM | Human aorta caldesmon mRNA, complete cds. |
| HUMHTF4A | Homo sapiens transcription factor (HTF4A) mRNA, complete cds. |
| HUMFPRL1A | Human formyl peptide receptor-like receptor (FPRL1) mRNA, |
| | complete |
| HUMOPIODRE | Human putative opioid receptor mRNA, complete cds. |
| HUMMGDBEA | Human glycogen debranching enzyme mRNA, complete cds. |
| HUMACTN2A | Homo sapiens skeletal muscle alpha 2 actinin (ACTN20 mRNA, |
| | complete) |
| HUMKSAMI | Human fibroblast growth factor receptor (K-sam) mRNA, complete |
| | cds. |
| HUMPAX2A | Human paired-box protein (PAX2) mRNA, complete cds. |
| HUMCYCLOX | Homo sapiens cyclooxygenase-2 (Cox-2) mRNA, complete cds. |
| HUMCCND3A | Human D3-type cyclin (CCND3) mRNA, complete cds. |
| HUMCYCD3A | Homo sapiens cyclin D3 (CCND3) mRNA, complete cds. |
| HUMHOX2A | Human homeobox 2.1 protein (HOX2A) mRNA, complete cds. |
| HUMG0S24A | H. sapiens zinc finger transcriptional regulator mRNA, complete cds. |
| HUMIA1X | Human zinc-finger DNA-binding motifs (IA-1) mRNA, complete |
| | cds. |
| HUMCYP7 | Human cholesterol 7-alpha hydroxylase (CYP7) mRNA, complete |
| | cds. |
| HUMPTPRZ | Human protein tyrosine phosphatase zeta-polypeptide (PTPRZ) |
| | mRNA, |
| HUMPLCB2A | Homo sapiens phospholipase C-beta-2 mRNA, complete cds. |
| HUMID2B | Human striated muscle contraction regulatory protein (Id2B) |
| | mRNA, |
| HUMNUCTIAR | Homo sapiens nucleolysin TIAR mRNA, complete cds. |
| HUMKALL | Homo sapiens Kallmann syndrome (KAL) mRNA, complete cds. |
| HUMGOGG | Human beta-1,6-N-acetylglucosaminyltransferase mRNA, complete |
| | cds. |
| HUMPDE2A | Human rolipram-sebsitive, cAMP-specific phosphodiesterase |
| | (PDE2) |
| S40706 | GADD153 = growth arrest and DNA-damage-inducible gene |
| | [human] |
| S46622 | calcineurin A catalytic subunit [human, testis, mRNA, 2134 nt]. |
| S59184 | RYK = related to receptor tyrosine kinase [human, hepatoma, |
| | mRNA, |
| S62138 | TLS/CHOP = hybrid gene {translocation breakpoint} [human, |
| | myxoid] |
| S66427 | RBP1 = retinoblastoma binding protein 1 [human, Nalm-6 pre-B cell] |
| S67044 | CD36 = collagen type I/thrombospondin receptor {one exon} |
| | [human] |
| S70004 | glycogen synthase [human, liver, mRNA, 2912 nt]. |
| S73498 | AgX-1 antigen [human, infertile patient, testis, mRNA, 2279 nt]. |
| S75881 | A-myb = DNA-binding transactivator {3′ region} [human, CCRF- |
| | CEM] |
| S76473 | trkB [human, brain, mRNA, 3194 nt]. |
| S77770 | voltage-gated chloride channel [human, placenta, Genomic/mRNA, |
| | 3440] |
| S78234 | nuc2 homolog [human, fibroblasts, mRNA, 3320 nt]. |
| S79851 | thioredoxin reductase [human, placenta, mRNA, 3826 nt]. |
| S82081 | N8 = tumor expression-enhanced gene [human, NCI H-69 cell line, |
| | mRNA] |
| S82592 | Evi-1 = Evi-1 protein {3′ region, deletion region} [human] |
| S82692 | interleukin-2 [human, placenta, term placentas obtained by cesarean |
| S82986 | HOXC6 = homeodomain-containing protein {clone 211} [human, |
| | MCF7] |
| S83309 | germ cell nuclear factor [human, embryonal carcinoma NT2/D1, |
| | mRNA] |
| U00238 | Homo sapiens glutamine PRPP amidotransferase (GPAT) mRNA, |
| | complete |
| U00672 | Human interleukin-10 receptor mRNA, complete cds. |
| HSU02081 | Human guanine nucleotide regulatory protein (NET1) mRNA, |
| | complete |
| HSU02882 | Human rolipram-sensitive 3′,5′-cyclic AMP phosphodiesterase |
| | mRNA, |
| HSU03272 | Human fibrillin-2 mRNA, complete cds. |
| HSU03688 | Human dioxin-inducible cytochrome P450 (CYP1B1) mRNA, |
| | complete cds. |
| HSU04313 | Human maspin mRNA, complete cds. |
| HSU04840 | Human onconeural ventral antigen-1 (Nova-1) mRNA, complete |
| | cds. |
| HSU06233 | Human POU domain protein (Brn-3b) mRNA, complete cds. |
| HSU07132 | Human steroid hormone receptor Ner-I mRNA, complete cds. |
| HSU07559 | Human ISL-1 (Islet-1) mRNA, complete cds. |
| HSU07681 | Human NAD(H)-specific isocitrate dehydrogenase alpha subunit |
| HSU07919 | Human aldehyde dehydrogenase 6 mRNA, complete cds. |
| HSU08023 | Human cellular proto-oncogene (c-mer) mRNA, complete cds. |
| HSU08839 | Human urokinase-type plasminogen activator receptor mRNA, |
| | complete |
| HSU09564 | Human serine kinase mRNA, complete cds. |
| HSU10301 | Human glutamate receptor flip isoform (GluR3-flip) mRNA, |
| | complete |
| HSU10417 | Homo sapiens ileal sodium-dependent bile acid transporter |
| HSU11058 | Homo sapiens calcium dependent potassium channel alpha subunit |
| HSU11287 | Human N-methyl-D-aspartate receptor subunit NR3 (hNR3) |
| | mRNA, |
| HSU11700 | Human copper transporting ATPase mRNA, complete cds. |
| HSU12128 | Human protein tyrosine phosphatase 1E (PTP1E) mRNA, complete |
| | cds. |
| HSU12140 | Human tyrosine kinase receptor p145TRK-B (TRK-B) mRNA, |
| | complete |
| HSU12535 | Human epidermal growth factor receptor kinase substrate (Eps8) |
| HSU12767 | Human mitogen induced nuclear orphan receptor (MINOR) mRNA, |
| HSU13047 | Human nuclear respiratory factor-2 subunit gamma 1 mRNA, |
| | complete |
| HSU13048 | Human nuclear respiratory factor-2 subunit gamma 2 mRNA, |
| | complete |
| HSU13219 | Human forkhead protein FREAC-1 mRNA, complete cds. |
| HSU13913 | Human large-conductance calcium-activated potassium channel |
| | (hSlo) |
| HSU14193 | Human TFIIA gamma subunit mRNA, complete cds. |
| HSU14391 | Human myosin-IC mRNA, complete cds. |
| HSU14407 | Human interleukin 15 (IL15) mRNA, complete cds. |
| HSU16307 | Human glioma pathogenesis-related protein (GliPR) mRNA, |
| | complete |
| HSU16954 | Human (AF1q) mRNA, complete cds. |
| U17195 | Homo sapiens A-kinase anchor protein (AKAP100) mRNA, |
| | complete cds. |
| HSU17714 | Homo sapiens putative tumor suppressor ST13 (ST13) mRNA, |
| | complete |
| HSU17989 | Homo sapiens nuclear autoantigen GS2NA mRNA, complete cds. |
| HSU18259 | Human clone CIITA-8 MHC class II transactivator CIITA mRNA, |
| HSU18423 | Human spinal muscular atrophy gene product mRNA, complete cds. |
| HSU18914 | Human 19.8 kDa protein mRNA, complete cds. |
| HSU19252 | Human putative transmembrane protein mRNA, complete cds. |
| HSU19878 | Human transmembrane protein mRNA, complete cds. |
| HSU20362 | Human Tg737 mRNA, complete cds. |
| HSU22680 | Human X2 box repressor mRNA, complete cds. |
| HSU23851 | Human atrophin-1 mRNA, complete cds. |
| HSU24183 | Human phosphofructokinase (PFKM) mRNA, complete cds. |
| HSU25676 | Human interleukin 2 (IL2) mRNA, complete cds. |
| HSU25997 | Homo sapiens stanniocalcin precursor (STC) mRNA, complete cds. |
| HSU26312 | Human heterochromatin protein HP1Hs-gamma mRNA, complete |
| | cds. |
| HSU26424 | Human Ste20-like kinase (MST2) mRNA, complete cds. |
| HSU27193 | Human protein-tyrosine phosphatase mRNA, complete cds. |
| HSU27655 | Human RGP3 mRNA, complete cds. |
| HSU28687 | Human zinc finger containing protein ZNF157 (ZNF157) mRNA, |
| | complete |
| HSU28926 | Human beta2-chimaerin mRNA, complete cds. |
| HSU29165 | Human MOP1 mRNA, complete cds. |
| HSU31383 | Human G protein gamma-10 subunit mRNA, complete cds. |
| HSU32500 | Human type 2 neuropeptide Y receptor mRNA, complete cds. |
| HSU32659 | Human IL-17 mRNA, complete cds. |
| HSU34605 | Human retinoic acid- and interferon-inducible 58 K protein RI58 |
| HSU37426 | Human kinesin-like spindle protein HKSP (HKSP) mRNA, |
| | complete cds. |
| HSU37449 | Human Mch3 isoform beta (Mch3) mRNA, complete cds. |
| HSU37529 | Human substance P beta-PPT-A mRNA, complete cds. |
| HSU37546 | Human IAP homolog C (MIHC) mRNA, complete cds. |
| HSU37707 | Human dlg3 mRNA, complete cds. |
| HSU38654 | Homo sapiens Rab27a mRNA, complete cds. |
| HSU38810 | Human mab-21 cell fate-determining protein homolog (CAGR1) |
| | mRNA, |
| HSU39196 | Human clone hGIRK1 G-protein coupled inwardly rectifying |
| | potassium |
| HSU39657 | Human MAP kinase kinase 6 (MKK6) mRNA, complete cds. |
| HSU40281 | Human cysteine protease CMH-1 mRNA, complete cds. |
| HSU40343 | Human CDK inhibitor p19INK4d mRNA, complete cds. |
| HSU41766 | Human metalloprotease/disintegrin/cysteine-rich protein precursor |
| HSU41816 | Human C-1 mRNA, complete cds. |
| HSU42766 | Human neuropeptide y2 receptor mRNA, complete cds. |
| HSU43030 | Human cardiotrophin-1 (CTF1) mRNA, complete cds. |
| HSU43142 | Human vascular endothelial growth factor related protein VRP |
| | mRNA, |
| HSU43653 | Human obese protein (ob) mRNA, complete cds. |
| HSU46024 | Homo sapiens myotubularin (MTM1) mRNA, complete cds. |
| HSU46573 | Human eotaxin precursor mRNA, complete cds. |
| HSU47741 | Human CREB-binding protein (CBP) mRNA, complete cds. |
| HSU48436 | Homo sapiens fragile X mental retardation protein FMR2p (FMR2) |
| HSU49184 | Human occludin mRNA, complete cds. |
| HSU49516 | Human serotonin 5-HT2c receptor mRNA, complete cds. |
| HSU49837 | Human LIM protein MLP mRNA, complete cds. |
| HSU49957 | Human LIM protein (LPP) mRNA, partial cds. |
| HSU50078 | Human guanine nucleotide exchange factor p532 mRNA, complete |
| | cds. |
| HSU50196 | Human adenosine kinase mRNA, complete cds. |
| HSU50928 | Human autosomal dominant polycystic kidney disease type II |
| | (PKD2) |
| HSU50929 | Human betaine: homocysteine methyltransferase mRNA, complete |
| | cds. |
| HSU50964 | Human G protein-activated inwardly rectifying potassium channel |
| HSU51166 | Human G/T mismatch-specific thymine DNA glycosylase mRNA, |
| | complete |
| HSU51333 | Human hexokinase III (HK3) mRNA, complete cds. |
| HSU52153 | Human inwardly rectifying potassium channel Kir3.2 mRNA, |
| | complete |
| HSU52426 | Homo sapiens GOK (STIM1) mRNA, complete cds. |
| HSU53476 | Human proto-oncogene Wnt7a mRNA, complete cds. |
| HSU56085 | Human periodic tryptophan protein 2 (PWP2) mRNA, complete cds. |
| HSU56236 | Human Fc alpha receptor b mRNA, complete cds. |
| HSU56237 | Human Fc alpha receptor b deltaS2 mRNA, complete cds. |
| HSU56417 | Human lysophosphatidic acid acyltransferase-alpha mRNA, |
| | complete |
| HSU56998 | Human putative serine/threonine protein kinase PRK (prk) mRNA, |
| HSU57627 | Human fetal brain oculocerebrorenal syndrome (OCRL1) mRNA, |
| | complete |
| HSU57629 | Human retinitis pigmentosa GTPase regulator (RPGR) mRNA, |
| | complete |
| HSU59269 | Human hyaluronan synthase mRNA, complete cds. |
| HSU60319 | Homo sapiens haemochromatosis protein (HLA-H) mRNA, |
| | complete cds. |
| HSU61234 | Human tubulin-folding cofactor C mRNA, complete cds. |
| HSU61276 | Human transmembrane protein Jagged 1 (HJ1) mRNA, complete |
| | cds. |
| HSU61500 | Human GT334 protein (GT334) gene mRNA, complete cds. |
| HSU61835 | Human cyclin G1 interacting protein (1500GX1) mRNA, complete |
| | cds. |
| HSU62136 | Homo sapiens enterocyte differentiation associated factor EDAF-1 |
| HSU62769 | Human oxytocinase variant 2 mRNA, complete cds. |
| HSU63970 | Human canalicular multispecific organic anion transporter |
| | (cMOAT) |
| HSU65785 | Human 150 kDa oxygen-regulated protein ORP150 mRNA, |
| | complete cds. |
| HSU67319 | Human Lice2 beta cysteine protease mRNA, complete cds. |
| HSU67320 | Human Lice2 gamma cysteine protease mRNA, complete cds. |
| HSU67615 | Human beige protein homolog (chs) mRNA, complete cds. |
| HSU68727 | Human homeobox-containing protein mRNA, complete cds. |
| HSU70136 | Human megakaryocyte stimulating factor mRNA, complete cds. |
| HSU70323 | Human ataxin-2 (SCA2) mRNA, complete cds. |
| HSU70426 | Homo sapiens A28-RGS14p mRNA, complete cds. |
| HSU71300 | Human snRNA activating protein complex 50 kD subunit (SNAP50) |
| | mRNA, |
| HSU72206 | Human guanine nucleotide regulatory factor (LFP40) mRNA, |
| | complete |
| HSU72671 | Human telencephalin precursor mRNA, complete cds. |
| HSU76420 | Human dsRNA adenosine deaminase DRADA2a (DRADA2a) |
| | mRNA, complete |
| HSU76421 | Human dsRNA adenosine deaminase DRADA2b (DRADA2b) |
| | mRNA, complete |
| HSU78575 | Human 68 kDa type I phosphatidylinositol-4-phosphate 5-kinase |
| | alpha |
| HSU79274 | Human clone 23733 mRNA, complete cds. |
| HSU79751 | Human basic-leucine zipper nuclear factor (JEM-1) mRNA, |
| | complete |
| HSU80191 | Human TFIID subunit TAFII100 mRNA, complete cds. |
| HSU80802 | Homo sapiens orphan nuclear receptor GCNF mRNA, complete cds. |
| HSU83192 | Homo sapiens post-synaptic density protein 95 (PSD95) mRNA, |
| HSU84007 | Human glycogen debranching enzyme isoform 1 (AGL) mRNA, |
| HSU84008 | Human glycogen debranching enzyme isoform 2 (AGL) mRNA, |
| HSU84009 | Human glycogen debranching enzyme isoform 3 (AGL) mRNA, |
| HSU84010 | Human glycogen debranching enzyme isoform 4 (AGL) mRNA, |
| HSU84011 | Human glycogen debranching enzyme isoform 6 (AGL) mRNA, |
| HSU84249 | Homo sapiens basic-leucine zipper transcription factor MafG |
| | (MAFG) |
| HSU84487 | Human CX3C chemokine precursor, mRNA, alternatively spliced |
| HSU84573 | Homo sapiens lysyl hydroxylase isoform 2 (PLOD2) mRNA, |
| | complete |
| HSU87309 | Human hVps41p (HVPS41) mRNA, complete cds. |
| HSU87460 | Human putative endothelin receptor type B-like protein mRNA, |
| HSU88323 | Human placental bone morphogenic protein PLAB mRNA, |
| | complete cds. |
| HSU89012 | Homo sapiens dentin matrix acidic phosphoprotein 1 (DMP1) |
| | mRNA |
| HSU90142 | Human unknown protein (BT2.1) mRNA, complete cds. |
| HSU90278 | Human N-methyl-D-aspartate receptor 2B subunit precursor |
| | mRNA, |
| HSU90543 | Human butyrophilin (BTF1) mRNA, complete cds. |
| HSU91618 | Human proneurotensin/proneuromedin N mRNA, complete cds. |
| HSU91835 | Human CX3C chemokine precursor, mRNA, alternatively spliced, |
| HSU91934 | Human retina-derived POU-domain factor-1 mRNA, complete cds. |
| HSU91935 | Human retina-derived POU-domain factor-1 mRNA, alternatively |
| HSU92459 | Human metabotropic glutamate receptor 8 mRNA, complete cds. |
| HSU93091 | Human Toll protein homolog mRNA, complete cds and LINE-1 |
| | reverse |
| HSU93869 | Human RNA polymerase III subunit (RPC39) mRNA, complete |
| | cds. |
| HSIFR14 | Messenger RNA for human leukocyte (alpha) interferon. |
| HSIFR9 | Messenger RNA for human leukocyte (alpha) interferon. |
| HSPGK1 | Human mRNA encoding phosphoglycerate kinase. |
| HSLYTR | Human mRNA for lymphotoxin. |
| HSTNFR | Human mRNA for tumor necrosis factor. |
| HSIL1AR | Human mRNA for interleukin 1-alpha. |
| HSCSIST | Human mRNA for c-sis gene (clone pSM-1). |
| HSIL1R | Human mRNA for interleukin-1 precursor (pre IL-1). |
| HSGCRAR | Human mRNA for alpha-glucocorticoid receptor (clone OB7). |
| HSTRKR | Human mRNA of trk oncogene. |
| HSATPBR | Human mRNA for Na/K-ATPase beta subunit. |
| HSLD78R | Human tonsillar lymphocyte LD78 mRNA induced by TPA or PHA |
| | TPA |
| HSBPGMR | Human erythrocyte 2,3-bisphosphoglycerate mutase mRNA EC |
| | 2.7.5.4. |
| HSIFNB2R | Human IFN-beta 2a mRNA for interferon-beta-2. |
| HSIL5R | Human mRNA for T-cell replacing factor (interleukin-5). |
| HSTHMOD | Human mRNA for thrombomodulin precursor. |
| HSTRK2H | Human mRNA for trk-2h oncogene. |
| HSRPHO2A | Human mRNA for protein phosphatase 2A (alpha-type). |
| HSI12SRN | Human 12S RNA induced by poly(rI), poly(rC) and Newcastle |
| | disease |
| HSLIF | Human mRNA for leukaemia inhibitory factor (LIF/HILDA). |
| HSCOL3AI | Human mRNA for pro-alpha-1 type 3 collagen. |
| HSGABAAA1 | Human mRNA for GABA-A receptor, alpha 1 subunit. |
| HSTS | Human mRNA for thrombospondin. |
| HSTM2CEA | Human mRNA for transmembrane carcinoembryonic antigen BGPb |
| HSMBPC | Human mRNA for mannose-binding protein C. |
| HSTM1CEA | Human mRNA for transmembrane carcinoembryonic antigen BGPa |
| HSNMTDC | Human mRNA for NAD-dependent methylene tetrahydrofolate |
| HSABL | Human c-abl mRNA encoding p150 protein. |
| HSHOX2H | Human HOX2H mRNA from the Hox2 locus. |
| HSINTAL4 | Human mRNA for integrin alpha-4 subunit. |
| HSINAL2 | Human mRNA for integrin alpha-2 subunit. |
| HSBCASR | Human mRNA for beta-casein. |
| HSNCHIM | Human mRNA for n-chimaerin. |
| HSET3AA | H. sapiens endothelin 3 mRNA. |
| HSUDGM | Human mRNA for uracil-DNA glycosylase. |
| HSBNGFAC | Human mRNA for beta nerve growth factor. |
| HSCGJP | Human mRNA for cardiac gap junction protein. |
| HSLAG1CDN | Human LAG-1 mRNA. |
| HSMIP2B | Human mRNA for macrophage inflammatory protein-2beta |
| | (MIP2beta). |
| HSFCREC | Human mRNA for Fc receptor. |
| HSCREBA | H. sapiens cDNA for CREB protein. |
| HSITKBI | H. sapiens mRNA for 1D-myo-inositol-trisphosphate 3-kinase B |
| HSPM5 | Human mRNA for pM5 protein. |
| HSSERR52 | H. sapiens serotonin 5-HT2 receptor mRNA. |
| HMAIF | Human mRNa for adipogenesis inhibitory factor. |
| HSD1DORE | Human mRNA for D-1 dopamine receptor. |
| HSD13S106 | Homo sapiens D13S106 mRNA for a highly charged amino acid |
| | sequence. |
| HSRR2SS | H. sapiens RR2 mRNA for small subunit ribonucleotide reductase. |
| HSZFX2 | Human ZFX mRNA for put. transcription activator, isoform 2. |
| HSZFX3 | Human ZFX mRNA for put. transcription activator, isoform 3. |
| HSPRAD1CY | Human PRAD1 mRNA for cyclin. |
| HSTCF1B | Human TCF-1 mRNA for T cell factor 1 (splice form B). |
| HSTCF1C | Human TCF-1 mRNA for T cell factor 1 (splice form C). |
| HSKALIG | H. sapiens KALIG-1 mRNA for neural cell adhesion and axonal |
| HSPTBMR | Human polypyrimidine tract-binding (PTB) mRNA for |
| | polypyrimidine |
| HSP2RNA | H. sapiens mRNA for P2 protein of peripheral myelin. |
| HSTRKT1 | H. sapiens mRNA (TRK-T1) for 55 KD protein. |
| HSRSRFC4 | Homo sapiens mRNA for serum response factor-related protein, |
| HSTRE210 | H. sapiens mRNA for tre oncogene (clone 210). |
| HSTRE213 | H. sapiens mRNA for tre oncogene (clone 213). |
| HSPMCATP | H. sapiens mRNA for plasma membrane calcium ATPase. |
| HSAPO1 | H. sapiens mRNA for APO-1 cell surface antigen. |
| HSRDC1MR | H. sapiens mRNA for RDC-1 POU domain containing protein. |
| HSDBT | H. sapiens mRNA for transacylase (DBT). |
| HSMATUMN | H. sapiens MaTu MN mRNA for p54/58N protein. |
| HSHNRNPI | H. sapiens mRNA for heterogeneous nuclear ribonucleoprotein. |
| HSCD40 | H. sapiens mRNA for CD40 ligand. |
| HSMEF2 | H. sapiens mRNA for myocyte-specific enhancer factor 2 (MEF2). |
| HSTRAPA | H. sapiens TRAP mRNA for ligand of CD40. |
| HSNC30 | H. sapiens interleukin-13 mRNA. |
| HSCALRE | H. sapiens mRNA for calcitonin receptor. |
| HSFMR1A | H. sapiens FMR-1 mRNA. |
| HSTGFAA | H. sapiens mRNA for transforming growth factor alpha. |
| HSFUSCPA | Homo sapiens mRNA for FUS-CHOP protein fusion. |
| HSERGICA | H. sapiens ERGIC-53 mRNA. |
| HSREVERB2 | H. sapiens mRNA encoding Rev-ErbAalpha (internal fragment). |
| HSNICE | H. sapiens mRNA for nicein B2 chain. |
| HSP130K | H. sapiens p130 mRNA for 130 K protein. |
| HSVHATPE | H. sapiens mRNA for vacuolar H+ ATPase E subunit. |
| HS25ABP | H. sapiens mRNA for 2-5A binding protein. |
| HSCATHO | H. sapiens mRNA for cathepsin-O. |
| HSGD3S | H. sapiens GD3 synthase mRNA. |
| HSHZF10 | H. sapiens HZF10 mRNA for zinc finger protein. |
| HSSCA1 | H. sapiens SCA1 mRNA for ataxin. |
| HSAUHMR | H. sapiens AUH mRNA. |
| HSERK3 | H. sapiens ERK3 mRNA. |
| HSARCP5 | H. sapiens mRNA (clone p5) for archain. |
| HSHTFIIAS | H. sapiens hTFIIAs mRNA for smallest (gamma) TFIIA subunit. |
| HSHE6 | H. sapiens mRNA for HE6 Tm7 receptor. |
| HSHOK2H1 | H. sapiens HOK-2 mRNA for zinc finger protein. |
| HSSTAF50 | H. sapiens Staf50 mRNA. |
| HSACENT | H. sapiens mRNA for alpha-centractin. |
| HSBRN4 | H. sapiens Brain 4 mRNA. |
| HSSMA4 | H. sapiens SMA4 mRNA. |
| HSIDEM | H. sapiens mRNA for phosphatidylinositol 3 kinase gamma. |
| HSAPXL | H. sapiens APXL mRNA. |
| HSRNACINP | H. sapiens mRNA for cytokine inducible nuclear protein. |
| HSEP3C | H. sapiens mRNA for prostaglandin E receptor (EP3c). |
| HSPKX1MR | H. sapiens mRNA for protein kinase, PKX1. |
| HSTRPC1GN | H. sapiens mRNA for TRPC1 protein. |
| HSRNAESM1 | H. sapiens mRNA for ESM-1 protein. |
| HSMTTP | H. sapiens mRNA for microsomal triglyceride transfer protein. |
| HSMHCIMIC | H. sapiens mRNA for MHC class I mic-B antigen. |
| HSNOV | H. sapiens mRNA for novel gene in Xq28 region. |
| HSTRAXGEN | H. sapiens mRNA for translin associated protein X. |
| HSPWP2GEE | H. sapiens mRNA for PWP2 protein. |
| HS14KDAPT | Homo sapiens mRNA for translational inhibitor protein p14.5. |
| HSTAFII10 | H. sapiens mRNA for TAFII100 protein. |
| HSC1DPROT | H. sapiens mRNA for C1D protein. |
| HSCMRP | H. sapiens mRNA for canalicular multidrug resistance protein. |
| HSTIM17 | H. sapiens mRNA for TIM17 preprotein translocase. |
| HSVITDITR | H. sapiens mRNA for protein induced by vitamin D. |
| HSCGM2ANT | H. sapiens mRNA for carcinoembryonic antigen family member 2, |
| | CGM2. |
| HSCH16FAA | H. sapiens mRNA for FAA protein. |
| HSBHLH | H. sapiens mRNA for B-HLH DNA binding protein. |
| HSRTRGCNF | H. sapiens mRNA for hRTR/hGCNF protein. |
| HSHAPRA | Human hap mRNA encoding a DNA-binding hormone receptor. |
| HSPAI2R | Human mRNA for Arg-Serpin (plasminogen activator-inhibitor 2) |
| HSHMPFK | Human mRNA for muscle phosphofructokinase (E.C.2.7.1.11). |
| HSMDNCF | Human mRNA for MDNCF (monocyte-derived neutrophil |
| | chemotactic factor) |
| HSIBP1R | Human mRNA for insulin-like growth factor binding protein IBP-1 |
| HS46KDA | H. sapiens mRNA for 46 kDa coxsackievirus and adenovirus |
| | receptor |
| HSSIRPBET | H. sapiens mRNA for SIRP-beta1. |
| HSWNT11 | Homo sapiens mRNA for WNT11 gene. |
| HSRIMP | Homo sapiens mRNA for FIM protein. |
| HSY13834 | Homo sapiens mRNA for farnesylated-proteins converting enzyme |
| | 1. |
| HSY13835 | Homo sapiens mRNA for farnesylated-proteins converting enzyme |
| | 2. |
| HSDIF2 | Homo sapiens mRNA for DIF-2 protein. |
| HSY15014 | Homo sapiens mRNA |
| HSY15228 | Homo sapiens mRNA for leukemia associated gene 2. |
| HSY16241 | Homo sapiens mRNA for nebulette. |
| HSRSRFC9 | Homo sapiens mRNA for serum response factor-related protein, |
| HSA16521 | Homo sapiens mRNA for CDS2 protein. |
| HSY16645 | Homo sapiens mRNA for monocyte chemotactic protein-2. |
| HSY17394 | Homo sapiens mRNA for prefoldin subunit 3. |
| HOS18206 | Homo sapiens mRNA for protein phosphatase 1 (PPP1R6). |
| HSNOCTPOU | H. sapiens mRNA for N-Oct 3, N-Oct5a, and N-Oct 5b proteins. |
| HSLAMB2T | H. sapiens mRNA for laminin. |
| HSALK2A | H. sapiens ALK-2 mRNA. |
| HSALK3A | H. sapiens ALK-3 mRNA. |
| HSA2CHIA | H. sapiens a2-chimaerin mRNA. |
| HSTROISOA | H. sapiens tropomyosin isoform mRNA, complete CDS. |
| HSUCEH3 | H. sapiens (23k/3) mRNA for ubiquitin-conjugating enzyme UbcH2. |
| HSINPO5P | H. sapiens mRNA for 43 kDa inositol polyphosphate 5-phosphatase. |
| HSXKMTP | Homo sapiens mRNA for membrane transport protein (XK gene). |
| HSCHK2 | H. sapiens HK2 mRNA for hexokinase II. |
| HSSOX9MRN | Homo sapiens SOX9 mRNA. |
| HSCTLA8 | H. sapiens CTLA8 mRNA. |
| HSPTPKAP | H. sapiens mRNA for phosphotyrosine phosphatase kappa. |
| HSTRPC1A | H. sapiens mRNA for TRPC1A. |
| HSCCCHK53 | H. sapiens mRNA for CC-chemokine, eotaxin variant (clone 53). |
| HS326L13 | Human DNA sequence from PAC 326L13 containing brain-4 |
| | mRNA ESTs |

What is claimed is

1. A method of selecting a set of nucleic acids for analyzing gene expression in a cell, said method comprising:
a) providing a database which comprises a plurality of nucleic acid sequences, each of said nucleic acid sequences comprising a full-length or partial length protein coding sequence and a 3′ untranslated region sequence downstream and contiguous with said protein coding sequence;
b) extracting a set of said protein coding sequences from said database by identifying protein coding sequences located upstream and contiguous with a 3′ untranslated region (UTR) which comprises one of the following target sequences:
i) a first target sequence, WU/T(AU/TU/TU/TA)U/TWWW, wherein none or one of the nucleotides outside of the parenthesis is replaced by a different nucleotide, and wherein W represents A, U, or T; or
ii) a second target sequence, U/T(AU/TU/TU/T)n wherein n indicates that the second target sequence comprises from 3 to 12 of the tetrameric sequences within the parenthesis.

2. The method of claim 1 wherein said database comprises mRNA sequences, cDNA sequences, or both.

3. The method of claim 1 wherein said database comprises genomic sequences.

4. The method of claim 1 wherein said database comprises genomic sequences, and
further comprising the step of excluding from said set the protein coding sequences of genes that have the target sequence in a region other than the 3′UTR.

5. A method of preparing a library of nucleic acid molecules for analyzing gene expression in a cell, comprising
a) obtaining a group of two or more nucleic acid molecules whose protein coding sequences have been selected according to the method of claim 1, wherein the protein coding sequence of each of said nucleic acid molecules is different from the protein coding sequences of the other nucleic acid molecules in said group, and
b) incorporating each of said nucleic acid molecules into a separate nucleic acid vector to provide the library.

6. The method of claim 5 further comprising the step of sequencing said nucleic acid molecules.

7. A nucleic acid library prepared according to the method of claim 5.

8. The nucleic acid library of claim 7 wherein the nucleic acid molecules comprise the coding sequences or a fragment thereof of the nucleic acids identified in Table 6.

9. The nucleic acid library of claim 8 wherein said library is substantially free of nucleic acid molecules whose protein coding sequences are contiguous with a 3′UTR which lacks a target sequence.

10. A method for preparing a customized array for analyzing expression of ARE genes in a cell, comprising
(a) determining the protein coding sequences of a plurality of the nucleic acid molecules selected according to the method of claim 1;
(b) attaching a gene probe for each of said nucleic acid molecules to a solid support to provide the array,
wherein each of said probes hybridizes under stringent conditions to a target region within said protein coding sequence or the complement thereof, and
wherein each of said probes is an oligonucleotide, cDNA molecule, or a synthetic gene probe which comprises nucleobases

11. A customized array prepared according to the method of claim 10.

12. The customized array of claim 11 wherein said array comprises a plurality of probes to the nucleic acids listed in Table 6.

13. The customized array of claim 12 wherein fewer than 20% of the probes on the array bind under stringent hybridization conditions to the protein coding sequences of non-ARE genes

14. The customized array of claim 12 wherein fewer than 10% of the probes on the array bind under stringent hybridization conditions to the protein coding sequences of non-ARE gene.

15. The customized array of claim 12 wherein the probes are oligonucleotides that are at least 10 nucleotides in length, wherein the GC content of said oligonucleotides is at least 40%, and wherein said oligonucleotides do not form hairpin structures.

16. The customized array of claim 11 wherein said protein coding sequences are selected by extraction from a genomic database.

17. A method of extracting ARE genes from a genomic database, comprising:
a) identifying genomic regions which comprise an an ARE motif;
b) locating the protein coding regions which are unstream of said genomic regions; and
c) subjecting the genomic regions located in step b to a computer gene prediction program which gives an output of the coding region and predicted amino acid sequence.

18. The method of claim 17 wherein the genomic areas are located by analyzing the genomic area located between 6 and 20 kilobases upstream and from 1 to 3 kilobases downstream of the ARE motif.

19. A method of using the nucleic acids selected by the method of claim 1 to prepare a customized array of ARE genes, comprising:
a) identifying a group of unique sequence within the protein coding sequence of the ARE genes selected according to claim 1;
b) preparing a set of oligonucleotides or polynucleotides, wherein each oligonucleotide or polynucleotide in said set comprises one of the unique sequences in said group; and
c) attaching said oligonucleotides or said polynucleotides to a solid support.

20. The method of claim 19 wherein the set of oligonucleoitdes or polynucleotides are prepared by
a) obtaining a DNA or RNA sample;
b) PCR amplifying said sample using primers which are specific for said unique sequences to provide said oligonucleotides or polynucleotides.

21. A method for identifying primer sets targeted to the initiation region of genes whose 3′ untranslated region comprise ARE sequences, comprising:
a) locating the start codon of the protein coding sequences of a plurality of genes whose 3′ UTR comprises one of the following target sequences:
i) a first target sequence, WU/T(AU/TU/TU/TA)U/TWWW, SEQ ID NO. 1 1, wherein none or one of the nucleotides outside of the parenthesis is replaced by a different nucleotide, and wherein W represents A, U, or T; or
ii) a second target sequence, U/T(AU/TU/TU/T)n, SEQ ID NO. 2, wherein n indicates that the second target sequence comprises from 3 to 12 of the tetrameric sequences within the parenthesis;
b) grouping said genes into a class selected from the group consisting of:
i) the ATGa genes whose initiation codon has an attached to the 3′ end,
ii) the ATGc genes whose initiation codon has a C attached to the 3′ end,
iii) the ATGg genes whose initiation codon has a G attached to the 3′ end, and
iv) the ATGt genes whose initiation codon has a T attached to the 3′ end; and
c) constructing a consensus sequence for each of said classes by analyzing the 9 nucleotides located immediately upstream of the initiation codon and the nucleotide located immediately downstream of the initiation codon,
wherein each of said consensus sequences is 13 nucleotides in length,
wherein each of said consensus sequences encompasses at least 75% of the genes in its related class, and
wherein the oligonucleotides encompassed by each of said four consensus sequences is a primer set.

22. The method of claim 21 wherein each of said consensus sequences encompasses the sequences of at least 90% of the genes in its related group.

23. A method for identifying primer sets targeted to the initiation region of genes whose 3′ untranslated region comprise ARE sequences, comprising:
a) locating the start codon of the protein coding sequences of a plurality of genes whose 3′ UTR comprises one of the following target sequences:
i) a first target sequence, WU/T(AU/TU/TU/TA)U/TWWW, SEQ ID NO. 1, wherein none or one of the nucleotides outside of the parenthesis is replaced by a different nucleotide, and wherein W represents A, U, or T; or
ii) a second target sequence, U/T(AU/TU/TU/T)n, SEQ ID NO. 2, wherein n indicates that the second target sequence comprises from 3 to 12 of the tetrameric sequences within the parenthesis;
b) grouping said genes into one of the following sixteen classes
i) the AATGa genes whose initiation codon, ATG, has an A attached to the 5′ end, and an A attached to the 3′ end,
ii) the CATGa genes whose initiation codon, ATG, has a C attached to the 5′ end, and an A attached to the 3′ end,
iii) the GATGa genes whose initiation codon, ATG, has a G attached to the 5′ end, and an A attached to the 3′ end,
iv) the TATGt genes whose initiation codon, ATG, has a T attached to the 5′ end, and an A attached to the 3′ end,
v) the AATGc genes whose initiation codon, ATG, has an A attached to the 5′ end, and an c attached to the 3′ end,
vi) the CATGc genes whose initiation codon, ATG, has a C attached to the 5′ end, and a c attached to the 3′ end,
vii) the GATGc genes whose initiation codon, ATG, has a G attached to the 5′ end, and a C attached to the 3′ end,
viii) the TAGc genes whose initiation codon, ATG, has a T attached to the 5′ end, and a C attached to the 3′ end,
ix) the ATGg genes whose initiation codon, ATG, has an A attached to the 5′ end, and a G attached to the 5′ end,
x) the CATGg genes whose initiation codon, ATG, has a C attached to the 5′ end, and a G attached to the 3′ end,
xi) the GATGg genes whose initiation codon, ATG, has a G attached to the 5′ end, and a G attached to the 3′ end,
xii) the TATGg genes whose initiation codon, ATG, has a T attached to the 5′ end, and a G attached to the 3′ end,
xiii) the ATGt genes whose initiation codon, ATG, has an A attached to the 5′ end, and a T attached to the 3′ end,
xiv) the CATGt genes whose initiation codon, ATG, has a C attached to the 5′ end, and a T attached to the 3′ end,
xv) the GATGt genes whose initiation codon, ATG, has a G attached to the 5′ end, and a T attached to the 3′ end, and
xvi) the TATGt genes whose initiation codon, ATG, has a T attached to the 5′ end, and a T attached to the 3′ end; and
c) constructing a consensus sequence for each of said classess by analyzing the 9 nucleotides located immediately upstream of the initiation codon and the single nucleotide located immediately downstream of the initiation codon,
wherein each of said consensus sequences is thirteen nucleotides in length and comprises the initiation codon and the nucleotide attached to the 3′ end thereof,
wherein each of said consensus sequences represents at least 75% of the genes in its related group, and
wherein the oligonucleotides encompassed by each of said sixteen consensus sequences is a primer set,

24. The method of claim 60 wherein each of said consensus sequences encompasses the sequences of at least 90% of the genes in its related group.

25. A method of selectively amplifying ARE-gene transcripts, said method comprising
a) reverse transcribing RNA molecules obtained from a cell which is expressing one or more ARE-genes using an oligo dT primer and a reverse transcriptase to provide a pool of single stranded DNA molecules;
b) amplifying a portion of the ARE-containing DNA molecules within said pool by a polymerase chain reaction which employs
i) a 3′ primer which is from 13 to 50 nucleotides in length and comprises from 2 to 10 pentamers having the sequence TAAAT, wherein said pentameric sequences are overlapping or non-overlapping;
i) and one or more of the primers encompassed by one of the 5′ primer sets obtained according the method of claim 21.

26. The method of claim 25 wherein said method employs two or more 5′primers whose sequences are encompassed by a consensus sequence selected from the group consisting of:

| | |
|---|---|
| VRVVRVVATGAV, | SEQ ID NO. 16 |
| VVVDRVBATGCH, | SEQ ID NO. 17 |
| VVBRVVATGGM, | SEQ ID NO. 18 |
| VDBVRHVATGTY,. | SEQ ID NO. 19 |

27. The method of claim 25 further comprising the step of sequencing the ARE-containing DNA molecules that are produced by step (b).

28. A method of preparing a library of nucleic acid molecules for analyzing gene expression in a cell comprising
a) obtaining a group of two or more nucleic acid molecules whose protein coding sequences have been identified according to the method of claim 27, wherein the protein coding sequence of each of said two or more nucleic acid molecules is different from the protein coding sequences of the other nucleic acid molecules in said group, and
b) incorporating each of said nucleic acid molecules into a separate nucleic acid vector to provide the library.

29. A nucleic acid library prepared according to the method of claim 28.

30. The nucleic acid library of claim 29 wherein the nucleic acid molecules comprise the coding sequences or a fragment thereof of the nucleic acid molecules identified in FIG. 7.

31. The nucleic acid library of claim 30 wherein said library is substantially free of nucleic acid molecules whose protein coding sequences are not contiguous with a 3′UTR which comprises the target sequence.

32. A method for preparing a customized array for analyzing expression of ARE genes in a cell, comprising
(a) determining the protein coding sequences of a plurality of ARE nucleic acid molecules amplified according to the method of claim 25;
(b) attaching a gene probe for each of said nucleic acid molecules to a solid support to provide the array,
wherein each of said probes hybridizes under stringent conditions to a target region within said protein coding sequence or the complement thereof, and
wherein each of said probes is an oligonucleotide, cDNA molecule, or a synthetic gene probe which comprises nucleobases.

33. A customized array prepared according to the method of claim 32.

34. The customized array of claim 33 wherein the probes are oligonucleotides that are at least 10 nucleotides in length, wherein the GC content of said oligonucleotides is at least 40%, and wherein said oligonucleotides do not form hairpin structures.

35. A customized array for analyzing expression of ARE-genes, wherein said array comprises a plurality of probes which bind under stringent conditions to nucleic acids which comprises the sequences listed in FIG. 7.

36. The customized array of claim 35 wherein fewer than 20% of the probes on the array bind under stringent hybridization conditions to the protein coding sequences of non-ARE genes

37. The customized array of claim 35 wherein fewer than 10% of the probes on the array bind under stringent hybridization conditions to the protein coding sequences of non-ARE gene.

38. The customized array of claim 35wherein the probes are oligonucleotides that are at least 10 nucleotides in length, wherein the GC content of said oligonucleotides is at least 40%, and wherein said oligonucleotides do not form hairpin structures.

39. A method of selectively amplifying ARE-gene transcripts, said method comprising
a) reverse transcribing RNA molecules obtained from a cell which is expressing one or more ARE-genes using an oligo dT primer and a reverse transcriptase to provide a pool of single stranded DNA molecules;
b) amplifying a portion of the ARE-containing DNA molecules within said pool by a polymerase chain reaction which employs
i) a 3′ primer which is from 13 to 50 nucleotides in length and comprises from 2 to 10 pentamers having the sequence TAAAT, wherein said pentameric sequences are overlapping or non-overlapping;
ii) and one or more of the primers encompassed by one of the 5′ primer sets obtained according the method of claim 23.

40. The method of claim 39 wherein said: method employs two or more 5′ primers whose sequences are encompassed by a consensus sequence selected from the group consisting of
BHDVMMAATGAV, SEQ ID NO. 20,
BSHMRVCATGAV, SEQ ID NO. 21,
HBVVRVGATGAD, SEQ ID NO. 22,
BDDVRHTATGAM, SEQ ID NO. 23
HDDVRBAATGCD, SEQ ID NO. 24,
VRSVRMCATGCB, SEQ ID NO. 25,
SSBBRMGATGCB, SEQ ID NO. 26,
VBDWWRTATGCM, SEQ ID NO. 27
VVBVRMAATGGV, SEQ ID NO. 28
VVVVRSCATGGM, SEQ ID NO. 29,
BVVSRVGATGGM, SEQ ID NO. 30
VDBHRBTATGGM, SEQ ID NO. 31
DRBVRMAATGTY, SEQ ID NO. 32
BVBMRYCATGTS, SEQ ID NO. 33,
VDBVRRGATGTY, SEQ ID NO. 34,
DVBVWDTATGTY, SEQ ID NO. 35 and combinations thereof.

41. The method of claim 39 further comprising the step of sequencing the ARE-containing DNA molecules that are produced by step (b).

42. A method of preparing a library of nucleic acid molecules for analyzing gene expression in a cell comprising
a) obtaining a group of two or more nucleic acid molecules whose protein coding sequences have been identified according to the method of claim 41, wherein the protein coding sequence of each of said two or more nucleic acid molecules is different from the protein coding sequences of the other nucleic acid molecules in said group, and
b) incorporating each of said nucleic acid molecules into a separate nucleic acid vector to provide the library.

43. A nucleic acid library prepared according to the method of claim 204.

44. The nucleic acid library of claim 43 wherein said library is substantially free of nucleic acid molecules whose protein coding sequences are not contiguous with a 3′UTR which comprises the target sequence.

45. A method for preparing a customized array for analyzing gene expression in a cell, comprising
(a) determining the protein coding sequences of a plurality of ARE nucleic acid molecules amplified according to the method of claim 39;
(b) attaching a gene probe for each of said nucleic acid molecules to a solid support to provide the array,
wherein said probe hybridizes under stringent conditions to a target region within said protein coding sequence or the complement thereof, and
wherein said probe is an oligonucleotide, cDNA molecule, or a synthetic gene probe which comprises nucleobases.

46. A customized array prepared according to the method of claim 45.

47. The customized array of claim 46 wherein fewer than 20% of the probes on the array bind under stringent hybridization conditions to the protein coding sequences of non-ARE genes

48. The customized array of claim 46 wherein fewer than 10% of the probes on the array bind under stringent hybridization conditions to the protein coding sequences of non-ARE gene.

49. The customized array of claim 46wherein the probes are oligonucleotides that are at least 10 nucleotides in length, wherein the GC content of said oligonucleotides is at least 40%, and wherein said oligonucleotides do not form hairpin structures.

50. A method of selectively amplifying ARE-gene transcripts, said method comprising
a) reverse transcribing RNA molecules obtained from a cell which is expressing one or more ARE-genes using a reverse transcriptase and an oligo dT primer that has an NH2 group at the 5′ end thereof to provide a pool of single stranded cDNA molecules;
b) ligating an oligmer to said cDNA molecules, said oligomer being from 50 to 70 nucleotides in length, said olibomer being phosphorylated at its 3′ end and protected at its 5′ end with an NH2, said oligomer having a sequence which does not hybridize under stringent conditions to human mRNA molecules;
d) PCR amplifying the ARE-containing DNA molecules within the cDNA molecules produced in step (c) by a polymerase chain reaction which employs
i) a 3′ primer which is from 13 to 50 nucleotides in length and comprises from 2 to 10 pentamers having the sequence TAAAT, wherein said pentameric sequences are overlapping or non-overlapping; and
ii) a 5′ primer whose sequence is identical to a sequence contained within the oligomer.

51. The method of claim 50 wherein the CG content of said 3′ primer is at least 40%.

52. The method of claim 50 further comprising the step of sequencing the ARE-containing DNA molecules that are produced by step (d).

53. A method of preparing a library of nucleic acid molecules for analyzing gene expression in a cell comprising
a) obtaining a group of two or more nucleic acid molecules whose protein coding sequences have been identified according to the method of claim 301, wherein the protein coding sequence of each of said two or more nucleic acid molecules is different from the protein coding sequences of the other nucleic acid molecules in said group, and
b) incorporating each of said nucleic acid molecules into a separate nucleic acid vector to provide the library.

54. A nucleic acid library prepared according to the method of claim 53.

55. The nucleic acid library of claim 54 wherein said library is substantially free of nucleic acid molecules whose protein coding sequences are not contiguous with a 3′UTR which comprises the target sequence.

56. A method for preparing a customized array for analyzing gene expression in a cell, comprising
(a) determining the protein coding sequences of a plurality of ARE nucleic acid molecules amplified according to the method of claim 50;
(b) attaching a gene probe for each of said nucleic acid molecules to a solid support to provide the array,
wherein said probe hybridizes under stringent conditions to a target region within said protein coding sequence or the complement thereof, and
wherein said probe is an oligonucleotide, cDNA molecule, or a synthetic gene probe which comprises nucleobases.

57. A customized array prepared according to the method of claim 56.

58. The customized array of claim 57 wherein fewer than 20% of the probes on the array bind under stringent hybridization conditions to the protein coding sequences of non-ARE genes

59. The customized array of claim 57 wherein fewer than 10% of the probes on the array bind under stringent hybridization conditions to the protein coding sequences of non-ARE gene.

60. The customized array of claim 57 wherein the probes are oligonucleotides that are at least 10 nucleotides in length, wherein the GC content of said oligonucleotides is at least 40%, and wherein said oligonucleotides do not form hairpin structures.

61. A method of selectively amplifying ARE-gene transcripts, said method comprising
a) reverse transcribing said RNA obtained from a cell for provie a pool of single-stranded DNA molecules, wherein said reverse transcription employs a reverse transcriptase and a 3′ primer which is from 13 to 50 nucleotides in length and comprises from 2 to 10 pentamers having the sequence TAAAT, wherein said pentameric sequences are overlapping or non-overlapping;
b) amplifying the ARE-containing DNA molecules within said pool by a polymerase chain reaction which employs
i) a 3′ primer which is from 15 to 50 nucleotides in length and comprises from 2 to 10 pentamers having the sequence TAAAT, wherein said pentameric sequences are overlapping or non-overlapping;
i) and one or more of the primers encompassed by one of the 5′ primer sets obtained according the method of claim 21.

62. The method of claim 61 wherein the reverse transcriptase is stable at 60° C.

63. The method of claim 61 wherein trehalose is included in the reverse transcription step.

64. The method of claim 61 wherein said method employs two or more 5′primers whose sequences are encompassed by a consensus sequence selected from the group consisting of:

| | |
|---|---|
| VRVVRVVATGAV, | SEQ ID NO. 16 |
| VVVDRVBATGCR, | SEQ ID NO. 17 |
| VVBRVVATGGM, | SEQ ID NO. 18 |
| VDBVRHVATGTY,. | SEQ ID NO. 19 |

65. The method of claim 61 further comprising the step of sequencing the ARE-containing DNA molecules that are produced by step (b).

66. A method of preparing a library of nucleic acid molecules for analyzing gene expression in a cell comprising
a) obtaining a group of two or more nucleic acid molecules whose protein coding sequences have been identified according to the method of claim 65, wherein the protein coding sequence of each of said two or more nucleic acid molecules is different from the protein coding sequences of the other nucleic acid molecules in said group, and
b) incorporating each of said nucleic acid molecules into a separate nucleic acid vector to provide the library.

67. A nucleic acid library prepared according to the method of claim 66.

68. The nucleic acid library of claim 67 wherein said library is substantially free of nucleic acid molecules whose protein coding sequences are not contiguous with a 3′UTR which comprises the target sequence.

69. A method for preparing a customized array for analyzing expression of ARE genes in a cell, comprising
(a) determining the protein coding sequences of a plurality of ARE nucleic acid molecules amplified according to the method of claim 61;
(b) attaching a gene probe for each of said nucleic acid molecules to a solid support to provide the array,
wherein each of said probes hybridizes under stringent conditions to a target region within said protein coding sequence or the complement thereof, and
wherein each of said probes is an oligonucleotide, cDNA molecule, or a synthetic gene probe which comprises nucleobases

70. A customized array prepared according to the method of claim 69.

71. The customized array of claim 70 wherein the probes are oligonucleotides that are at least 10 nucleotides in length, wherein the GC content of said oligonucleotides is at least 40%, and wherein said oligonucleotides do not form hairpin structures.

72. A method of selectively amplifying ARE-gene transcripts, said method comprising
a) reverse transcribing said RNA obtained from a cell for provie a pool of single-stranded DNA molecules, wherein said reverse transcription employs a reverse transcriptase and a 3′ primer which is from 13 to 50 nucleotides in length and comprises from 2 to 10 pentamers having the sequence TAAAT, wherein said pentameric sequences are overlapping or non-overlapping;
b) amplifying a portion of the ARE-containing DNA molecules within said pool by a polymerase chain reaction which employs
i) a 3′ primer which is from 13 to 50 nucleotides in length and comprises from 2 to 10 pentamers having the sequence TAAAT, wherein said pentameric sequences are overlapping or non-overlapping;
ii) and one or more of the primers encompassed by one of the 5′ primer sets obtained according the method of claim 23.

73. The method of claim 72 wherein said method employs two or more 5′primers whose sequences are encompassed by a consensus sequence selected from the group consisting of:
BHDVMMAATGAV, SEQ ID NO. 20,
BSHMRVCATGAV, SEQ ID NO. 21,
HBVVRVGATGAD, SEQ ID NO. 22,
BDDVRHTATGAM, SEQ ID NO. 23
HDDVRBAATGCD, SEQ ID NO. 24,
VRSVRMCATGCB, SEQ ID NO. 25,
SSBBRMGATGCB, SEQ ID NO. 26,
VBDWWRTATGCM, SEQ ID NO. 27
VVBVRMAATGGV, SEQ ID NO. 28
VVVVRSCATGGM, SEQ ID NO. 29,
BVVSRVGATGGM, SEQ ID NO. 30
VDBHRBTATGGM, SEQ ID NO. 31
DRBVRMAATGTY, SEQ ID NO. 32
BVBMRYCATGTS, SEQ ID NO. 33,
VDBVRRGATGTY, SEQ ID NO. 34,
DVBVWDTATGTY, SEQ ID NO. 35 and combinations thereof.

74. The method of claim 72 further comprising the step of sequencing the ARE-containing DNA molecules that are produced by step (b).

75. A method of preparing a library of nucleic acid molecules for analyzing gene expression in a cell comprising
a) obtaining a group of two or more nucleic acid molecules whose protein coding sequences have been identified according to the method of claim 72 wherein the protein coding sequence of each of said two or more nucleic acid molecules is different from the protein coding sequences of the other nucleic acid molecules in said group, and
b) incorporating each of said nucleic acid molecules into a separate nucleic acid vector to provide the library.

76. A nucleic acid library prepared according to the method of claim 75.

77. The nucleic acid library of claim 76 wherein said library is substantially free of nucleic acid molecules whose protein coding sequences are not contiguous with a 3′UTR which comprises the target sequence.

78. A method for preparing a customized array for analyzing gene expression in a cell, comprising
(a) determining the protein coding sequences of a plurality of ARE nucleic acid molecules amplified according to the method of claim 72;
(b) attaching a gene probe for each of said nucleic acid molecules to a solid support to provide the array,
wherein said probe hybridizes under stringent conditions to a target region within said protein coding sequence or the complement thereof, and
wherein said probe is an oligonucleotide, cDNA molecule, or a synthetic gene probe which comprises nucleobases.

79. A customized array prepared according to the method of claim 78

80. The customized array of claim 79 wherein fewer than 20% of the probes on the array bind under stringent hybridization conditions to the protein coding sequences of non-ARE genes

81. The customized array of claim 79wherein fewer than 10% of the probes on the array bind under stringent hybridization conditions to the protein coding sequences of non-ARE gene.

82. The customized array of claim 79 wherein the probes are oligonucleotides that are at least 10 nucleotides in length, wherein the GC content of said oligonucleotides is at least 40%, and wherein said oligonucleotides do not form hairpin structures.

83. A method of obtaining an ARE expression profile in a subject, comprising:
a) extracting RNA from a tissue sample obtained from the subject;
b) labeling said RNA with a detectable tag; and
c) contacting said labeled RNA with a microarray selected from the group consisting of the microarray of claim 11, the microarray of claim 33, the microarray of claim 46, the microarray of claim 57, the microarray of claim 70 and the microarray of claim 76.
d) determining the sequence or pattern of the labeled RNA molecules which hybridize under stringent conditions with the probes present on said microarray.
